(19) United States
(12) Patent Application Publication
(10) Pub. No.: US 2023/0414763 A1
(43) Pub. Date: Dec. 28, 2023

(54) TRANSMUCOSAL AMPHIPHILE-PROTEIN CONJUGATE VACCINE

(71) Applicant: Massachusetts Institute of Technology, Cambridge, MA (US)

(72) Inventors: Darrell J. Irvine, Arlington, MA (US); Brittany L. Hartwell, Chanhassen, MN (US)

(21) Appl. No.: 18/117,752

(22) Filed: Mar. 6, 2023

Related U.S. Application Data
(60) Provisional application No. 63/316,919, filed on Mar. 4, 2022.

(51) Int. Cl. A61K 47/54; A61K 39/12; A61K 39/39; A61K 47/10; A61P 31/18

(52) U.S. Cl. CPC A61K 47/544; A61K 39/12; A61K 39/39; A61K 47/10; A61P 31/18; A61K 2039/543

(57) ABSTRACT

What is disclosed is a vaccine comprising an immunogen conjugated to an albumin-binding polymer-lipid tail, wherein the vaccine is suitable for transmucosal (e.g, intranasal) administration. Also disclosed is a method of using the vaccine to immunize a subject by transmucosal (e.g, intranasal) administration of an effective amount of the vaccine, alone or with an adjuvant.

### REFERENCE TO RELATED APPLICATIONS

This application claims priority to U.S. Provisional Application No. 63/316,919, filed on Mar. 4, 2022. The entire contents of the aforementioned application are expressly incorporated herein by reference.

### STATEMENT REGARDING FEDERALLY SPONSORED RESEARCH

This invention was made with government support under AI144462 and AI048240 awarded by the National Institutes of Health. The government has certain rights in the invention.

### REFERENCE TO SEQUENCE LISTING

The instant application contains a Sequence Listing which has been submitted electronically in XML format and is hereby incorporated by reference in its entirety. Said XML copy, created on Jul. 3, 2023, is named 127299-03602.XML and is 5,605 bytes in size.

### INCORPORATION BY REFERENCE

All documents cited or referenced herein and all documents cited or referenced in the herein cited documents, together with any manufacturer's instructions, descriptions, product specifications, and product sheets for any products mentioned herein or in any document incorporated by reference herein, are hereby incorporated by reference, and may be employed in the practice of the invention.

### BACKGROUND

To combat long-standing epidemics such as HIV and emerging threats such as SARS-CoV-2, immunization strategies are needed that can elicit systemic antibody responses and humoral immunity at mucosal portals of entry in tandem [1-6]. Many pathogens including HIV, SARS-CoV-2, influenza, rotavirus, and cholera infect the host through mucosal surfaces and thus are thought to require engagement of both systemic and mucosal branches of the immune system, employing a combination of IgG and IgA antibodies, for effective management and protection [1, 6, 7]. Secretory IgA (SIgA) is the main humoral defense at mucosal tissue sites [4] and plays a particularly important role in providing protection through mechanisms such as immune exclusion, inhibition of transcytosis, and direct neutralization of virus [8, 9]. Establishment of antigen-specific SIgA antibodies at mucosal surfaces provides a frontline defense that can help prevent infection and transmission [10]. With HIV, where 90% of transmissions occur via mucosal routes, induction of mucosal IgA responses (in combination with systemic IgG) has been found to be effective in promoting protection against mucosal SHIV challenge in primates [11, 12]. Similarly, SARS-CoV-2 clinical studies have shown that mucosal IgA exhibits potent neutralization and is a strong correlate of protection against the virus, which primarily infects cells in the upper and lower respiratory mucosa [13, 14].

Traditional parenteral immunization regimens typically elicit poor mucosal immunity. By contrast, vaccination at mucosal surfaces, which initiates immune responses in mucosa associated lymphoid tissues (MALT), is known to be a very effective strategy to promote protective immunity at barrier tissues due to programming of mucosa-specific lymphocyte function and tissue homing at these sites [1, 3]. Priming of mucosal T and B lymphocytes takes place in MALT inductive sites, such as the nasal-associated lymphoid tissue (NALT) and gut-associated lymphoid tissue (GALT) [3, 15, 16]. Here, through a property of the ‘common mucosal immune system’, antigen priming can induce expression of homing markers that lead activated antigen specific T cells, B cells, and plasma cells to migrate to other local or distal mucosal effector sites [2, 3, 7, 17]. The location of antigen exposure determines which homing markers are expressed, dictating the homing destination and ultimate effector site. Typically, the strongest response is elicited at the site of antigen exposure and in the most anatomically adjacent mucosal tissue. For example, cells that experience antigen priming in the nasal-associated lymphoid tissue (NALT) acquire chemokine receptors (i.e., CCR10, α₄β₁) that can home to both the respiratory tract and genitourinary tract, such that intranasal immunization is able to establish humoral responses at both mucosal sites [2, 17].

Although well-motivated by the biology of mucosal immunity, delivery of vaccine components across mucosal barriers has been a major challenge for mucosal vaccine development [1-3]. Vaccine uptake into the underlying mucosal immune compartment is impeded by multiple factors, including potential rapid antigen loss due to degradation by proteolytic enzymes and acidic conditions at mucosal surfaces, high rates of mucociliary clearance, and the lack of diffusive uptake across the tight junctions of the epithelial monolayer [18-20]. In fact, only a small number of mucosal vaccines have reached licensure, all of which except the inactivated oral cholera vaccine are based on live attenuated pathogens that naturally infect mucosal surfaces, such as the oral polio vaccine (OPV) or the intranasal influenza type AB vaccine (FluMist) [3, 21, 22]. However, live attenuated vaccines often face manufacturing challenges, poor stability, and safety concerns. These challenges have been addressed in parenteral vaccines by a focus on recombinant protein- or polysaccharide-based subunit vaccines that are safe, stable, and highly manufacturable, but subunit vaccines have historically exhibited poor immunogenicity and short-lived responses when applied to mucosal barriers due in large part to challenges of delivery and poor uptake [3]. Development of technologies to overcome barriers to mucosal delivery while meeting safety and efficacy requirements of prophylactic vaccines remains an urgent unmet need.

### SUMMARY

This Summary introduces a selection of concepts in simplified form that are described further below in the Detailed Description. This Summary neither identifies key or essential features, nor limits the scope, of the claimed subject matter.

In one aspect, the present disclosure provides a vaccine comprising an amphiphilic conjugate, wherein the amphiphilic conjugate comprises an immunogen operably linked to an albumin-binding lipid, and wherein the vaccine is suitable for transmucosal administration to induce a humoral immune response.

In some embodiments, the transmucosal administration is intranasal administration.

In some embodiments, the immunogen is a protein antigen having a molecular weight between about 10 kDa and about 500 kDa.

In some embodiments, the immunogen comprises a protein antigen selected from the group consisting of a human immunodeficiency virus (HIV) antigen, a SARS-CoV-2 antigen, an influenza antigen, a rotavirus antigen, a cytomegalovirus (CMV) antigen, an Epstein-Barr virus (EBV) antigen, a respiratory syncytial virus (RSV) antigen, and a cholera antigen.

In some embodiments, the immunogen comprises a monomer antigen or trimer antigen.

In some embodiments, the immunogen comprises an antigenic peptide.

In some embodiments, the albumin-binding lipid is selected from the group consisting of a cholesterol, monoacyl lipid, and diacyl lipid. In some embodiments, the albumin-binding lipid is a diacyl lipid. In some embodiments, the albumin-binding lipid is 1,2-distearoyl-sn-glycero-3-phosphoethanolamine (DSPE).

In some embodiments, the immunogen is operably linked to the albumin-binding lipid via a first linker. In some embodiments, the first linker is selected from the group consisting of a hydrophilic polymer, a string of hydrophilic amino acids, polysaccharides, oligonucleotides, or a combination thereof. In some embodiments, the first linker comprises a polyethylene glycol (PEG) linker. In some embodiments, the first linker comprises 45 to 150 repeating units of PEG monomers. In some embodiments, the first linker comprises a PEG2K linker.

In some embodiments, the vaccine further comprising a second linker, wherein the second linker is located between the immunogen and the first linker, or between the albumin-binding lipid and the first linker. In some embodiments, the second linker comprises a PEG linker. In some embodiments, the second linker comprises 2 to 20 repeating units of PEG monomers. In some embodiments, the second linker comprises 4 repeating units of PEG monomers. In some embodiments, the second linker comprises a dibenzocyclooctyne (DBCO) group covalently conjugated to the repeating unit of PEG monomers.

In some embodiments, the immunogen comprises an HIV antigen. In some embodiments, the HIV antigen comprises HIV gp120 engineered outer domain-germ line-targeting immunogen 8 (eOD-GT8).

In some embodiments, the immunogen comprises a SARS-CoV-2 antigen. In some embodiments, the SARS-CoV-2 antigen comprises an antigen from the receptor-binding domain (RBD) of SARS-CoV-2 spike protein.

In some embodiments, the vaccine further comprises an adjuvant. In some embodiments, the adjuvant is selected from the group consisting of bis-(3′-5′)-cyclic dimeric guanosine monophosphate (cdGMP) and saponin monophosphoryl-lipid-A (MPLA) nanoparticle adjuvant (SMNP).

In some embodiments, transmucosal administration of the vaccine elicits or enhances production of antibodies that bind to the immunogen. In some embodiments, the antibodies comprise IgA antibodies, IgG antibodies, or IgA and IgG antibodies. In some embodiments, the antibodies are neutralizing antibodies.

In another aspect, the present disclosure provides a method of vaccinating a subject, comprising transmucosally administering to the subject an effective amount of a vaccine of the disclosure, thereby vaccinating the subject.

In yet another aspect, the present disclosure provides a method of immunizing a subject, comprising transmucosally administering to the subject an effective amount of a vaccine of the disclosure, thereby immunizing the subject.

In some embodiments, the vaccine is administered intranasally to the subject.

In some embodiments, the vaccine is administered in more than one dose. In some embodiments, doses of the vaccine are administered about 2, 4, 6 or 8 weeks apart. In some embodiments, the vaccine is administered at 0, 8, 16, and 24 weeks. In some embodiments, the vaccine is administered at a dose of about 5 μg to about 300 μg. In some embodiments, the vaccine is administered at a dose of about 50 μg, 100 μg, or 150 μg.

In some embodiments, the vaccine is administered in combination with an adjuvant. In some embodiments, the adjuvant comprises SMNP. In some embodiments, the SMNP is administered at a dose of about 5 μg to about 500 μg. In some embodiments, the SMNP is administered at a dose of about 300 μg, 375 μg or 450 μg. In some embodiments, the adjuvant comprises cdGMP. In some embodiments, the cdGMP is administered at a dose of about 25 μg to about 500 μg.

The following Detailed Description references the accompanying drawings which form a part this application, and which show, by way of illustration, specific example implementations. Other implementations may be made without departing from the scope of the disclosure.

### BRIEF DESCRIPTION OF THE DRAWINGS

FIGS. 1A-1G show synthesis of albumin-binding amphiphile-protein immunogen conjugates. FIG. 1A: Schematic of amph-eOD structure. FIG. 1B: Dynamic light scattering analysis of eOD and amph-eOD. FIG. 1C: SEC profile of eOD versus amph-eOD. (D) AF647-eOD or AF647-amph-eOD protein were incubated with albumin-functionalized agarose resin at 37° C., and the quantity of each protein bound to the resin after 2 h was quantified. Statistical significance determined by unpaired t-test. FIGS. 1E-1G: Fluorescent eOD or amph-eOD were incubated with murine C57Bl/6 splenocytes for 1 h at 37° C. at a range of concentrations, then washed and stained with fluorescent VRC01 antibody: FIG. 1E: Representative flow cytometry plots of eOD/amph-eOD and VRC01 binding to the cells. FIG. 1F: Percentage of cells positive for eOD alone or double positive for eOD and VRC01; statistical significance determined by two-way ANOVA followed by Sidak's post-hoc test. FIG. 1G: Mean fluorescence intensity (MFI) of eOD and VRC01 as a function of eOD concentration; statistically significant non-zero slope determined by simple linear regression. All data showing mean±s.e.m. (*p<0.05, **p<0.01, ***p<0.001, ****p<0.0001).

FIGS. 2A-2H show that amph-protein conjugates exhibit enhanced persistence in the nasal mucosa and transport across the mucosal surface. FIG. 2A: Schematics illustrating (top) ventral view of mouse upper palate and underside of top jaw, showing ROI used to quantify IVIS signals in FIG. 2B and FIG. 2E and (bottom) sagittal view of mouse skull and nasal cavity showing approximate location of corresponding coronal cross-sections used for histology in FIGS. 2G-2H. FIG. 2B: Representative IVIS images of fluorescent signal in the nasal cavity of BALB/c mice (n=3 animals/group) over time following i.n. administration of 5 μg AF647-eOD or AF647-amph-eOD mixed with 5 μg saponin monophosphoryl-lipid-A (MPLA) nanoparticle adjuvant (SMNP) adjuvant. Region of interest (ROI) used to quantify IVIS signal is marked with dotted white oval. FIG. 2C: Quantified IVIS signal from FIG. 2B in nasal cavity over time. Statistical significance determined by unpaired t-test. Data shown from one representative of two independent experiments. FIG. 2D: Quantified IVIS signal area under the curve (AUC, total radiance x time) from FIG. 2C. Statistical significance determined by unpaired t-test. FIG. 2E: Representative IVIS images showing vaccine uptake and retention in nasal cavity over time following intranasal administration of 5 μg AF647-eOD or AF647-amph-eOD mixed with 5 μg SMNP adjuvant in WT C57Bl/6 vs. FcRn−/− mice (n=3 animals/group). FIG. 2F: Quantified IVIS signal from FIG. 2E in nasal cavity of WT vs. FcRn−/− mice at 6 h. Statistical significance determined by two-way ANOVA followed by Tukey's post-hoc test. FIG. 2G: Representative histology images of vaccine in nasal cavity in WT vs. FcRn−/− mice at 6 h. Images in (ii) are higher magnification views of dashed areas marked in (i). Scale bars represent (i) 1 mm, (ii) 500 μm. FIG. 2H: Representative histology images of vaccine in nasal cavity in WT vs. FcRn−/− mice at 24 h. Images in (ii) are higher magnification views of dashed areas noted in (i). (iii) shows high magnification views stained with DAPI to identify the epithelial cell barrier. ‘e’ marks epithelium, ‘lp’ marks lamina propria, and ‘m’ marks mucus. Scale bars represent (i) 1 mm, (ii) 500 μm, (iii) 100 μm. All data showing mean±s.e.m. (*p<0.05, **p<0.01, ***p<0.001, ****p<0.0001).

FIGS. 3A-3H show that amph-protein conjugates prime enhanced GC B cell and Tfh responses in the NALT in an FcRn-dependent manner. FIGS. 3A-3D: Groups of BALB/c mice (n=5 animals/group) were immunized intranasally with 10 μg AF647-amph-eOD or AF647-eOD mixed with 5 μg saponin adjuvant, and NALT tissue was isolated 1 or 4 days later for flow cytometry analysis of antigen uptake: FIG. 3A: Schematics illustrating (i) NALT tissue location and (ii) experimental timeline. FIGS. 3B-3D: Representative flow cytometry plots of eOD signal gating and mean fluorescence intensities in (FIG. 3B) F4/80+ macrophages, (FIG. 3C) B cells, and (FIG. 3D) CD11c+ dendritic cells. Statistical significance determined by unpaired t-tests. FIGS. 3E-3H: Groups of C57Bl/6 (WT) or FcRn−/− mice (n=5 animals/group) were immunized with 5 μg eOD or amph-eOD mixed with 5 μg saponin adjuvant, and GC/Tfh responses were analyzed by flow cytometry on day 12: (FIG. 3E) schematic showing experimental timeline, (FIG. 3F) representative flow cytometry gating and enumeration of total GC B cells, (FIG. 3G) antigen-specific GC B cells, and (FIG. 3H) Tfh cells. Data shown from one representative of two independent experiments. Statistical significance determined by ordinary one-way ANOVA followed by Tukey's post-hoc test. All data showing mean±s.e.m. (*p<0.05, **p<0.01, **p<0.001, ****p<0.0001).

FIGS. 4A-4J show that amph-protein conjugates elicit enhanced systemic and mucosal immune responses following intranasal vaccination. FIGS. 4A-4E: BALB/c mice (n=5 animals/group) were immunized i.n. with 5 μg eOD or amph-eOD mixed with 25 μg cdGMP adjuvant and boosted 6 weeks later with the same formulations: FIG. 4A schematic illustrating the experimental timeline; IgG and IgA titers in the (FIG. 4B) serum, (FIG. 4C) vaginal wash, and (FIG. 4D) feces; FIG. 4E: FRT and BM eOD-specific IgA antibody-secreting cells assessed by ELISPOT one year post immunization. Data shown from one representative of two independent experiments. FIGS. 4F-4J: BALB/c mice (n=5 animals/group) were immunized with 5 μg eOD or amph-eOD mixed with 5 μg SMNP adjuvant and boosted 6 weeks later with the same formulations: FIG. 4F: schematic illustrating the experimental timeline; IgG and IgA titers in the (FIG. 4G) serum, (FIG. 4H) vaginal wash, and (FIG. 4I) feces; FIG. 4J: FRT and BM eOD-specific IgA antibody-secreting cells assessed by ELISPOT 35 wks post immunization. Data shown from one representative of two independent experiments. Statistical significance in FIG. 4E and FIG. 4J was determined by unpaired t-test, and in FIGS. 4B-4D and FIGS. 4G-4I was determined by ordinary two-way ANOVA followed by Sidak's post-hoc test (*p<0.05, **p<0.01, ***p<0.001, ****p<0.0001). All data showing mean±s.e.m.

FIGS. 5A-5F show that amph-RBD conjugate elicits enhanced systemic and mucosal neutralizing antibody responses to SARS-CoV-2 immunogens following intranasal vaccination. FIG. 5A: Schematic of amph-RBD structure. FIGS. 5B-5F: BALB/c mice (n=5 animals/group) were immunized i.n. with 5 μg RBD or amph-RBD mixed with 5 μg SMNP adjuvant and boosted 4 weeks later with the same formulations: FIG. 5B: schematic illustrating the experimental timeline; (FIG. 5C) IgG titers and (FIG. 5D) IgA titers in the serum, vaginal wash, fecal wash, saliva, nasal wash, and bronchoalveolar lavage fluid (BALF) at 6 wks; FIG. 5E: ACE2:RBD binding inhibition (IC50) of antibodies in serum and BALF at 6 wks; FIG. 5F: pseudovirus neutralizing antibody (NAb) titers (NT50) in the serum, nasal wash, and BALF at 6 wks. Dotted line represents the limit of quantitation. Data shown from one representative of two independent experiments. Statistical significance in FIGS. 5C-5D was determined by two-way ANOVA followed by Sidak's post-hoc test, and (E-F) determined by unpaired t-test (*p<0.05, **p<0.01, ***p<0.001, ****p<0.0001). All data showing mean±s.e.m.

FIGS. 6A-6E show that intranasal immunization with amph-protein conjugates leads to improved humoral immune responses in non-human primates. FIG. 6A: Rhesus macaques (n=3 animals/group) were immunized i.n. with 100 μg AF647-eOD or AF647-amph-eOD mixed with 375 μg SMNP adjuvant. Shown is quantified fluorescence signal of vaccine immunogens in the nasal cavity after 24 h by IVIS imaging. Statistical significance determined by unpaired t-test. (BE) Rhesus macaques (n=6 animals/group) were immunized i.n. with 100 μg eOD or amph-eOD mixed with 375 μg SMNP adjuvant and boosted at 8, 16, and 24 wks with the same formulations. FIG. 6B: schematic illustrating the experimental timeline. FIG. 6C: frequencies of antigen-specific IgM, IgG, and IgA secreting plasma blasts in peripheral blood as determined by ELISPOT. FIG. 6D: IgG and IgA titers in the serum over time, and individual animal IgG titers at 6 wks (middle panel; left: Amph-eOD, right: eOD). FIG. 6E: IgG and IgA titers in the nasal wash over time. Statistical significance in (C-E) determined by two-way ANOVA (*p<0.05, **p<0.01, ***p<0.001, ****p<0.0001). All data showing mean±s.e.m.

FIGS. 7A-7B show synthesis of amphiphile-protein conjugates. FIG. 7A: Sequence of eOD protein with PADRE peptide underlined. FIG. 7B: Reaction scheme for preparation of amph-eOD antigen conjugate.

FIGS. 8A-8C show amph-protein conjugate insertion into cell membranes. AF647-labeled eOD or amph-eOD were incubated with murine C57Bl/6 splenocytes for 1 h at 37° C. at a range of concentrations, washed and stained with Rhodamine-labeled VRC01 antibody, and evaluated by flow cytometry to assess eOD vs amph-eOD cell membrane insertion: FIG. 8A: Gating strategy for identification of VRC01+ and AF647+ cells. FIG. 8B: representative flow cytometry plots of eOD/amph-eOD and VRC01 binding to the cells at varying concentrations of eOD. FIG. 8C: ELISA measurements are shown for human serum albumin binding to plate-bound human FcRn in the presence of varying concentrations of DSPE-PEG2K-FITC

FIGS. 9A-9C show systemic distribution of amph-protein conjugates in mice. BALB/c mice (n=3 animals/group) were immunized intranasally with 5 μg AF647-eOD or AF647-amph-eOD mixed with 5 μg SMNP adjuvant, and tissues were collected after 24 h for IVIS analysis of AF647 fluorescent signal to evaluate systemic dissemination and distal lymphatic drainage of eOD vs amph-eOD: (FIG. 9A) Representative IVIS images and (FIG. 9B) quantified IVIS signal in the intestines, mesenteric lymph nodes (mLNs), cervical lymph nodes (cLNs), liver, and spleen after 24 h. Data showing mean±s.e.m. FIG. 9C: ELISA analysis is shown for albumin concentrations in the nasal wash of wild-type (WT) versus FcRn^{-/-} mice (n=5 animals per group). Statistical comparison was performed using Welch's t-test. All data showing mean±standard error of the mean (s.e.m.). ns, not significant.

FIG. 10 shows amph-protein uptake in mouse NALT cell populations. Groups of BALB/c mice (n=5 animals/group) were immunized intranasally with 10 μg AF647-amph-eOD or AF647-eOD mixed with 5 μg SMNP adjuvant, and NALT tissue was isolated 1 or 4 days later for flow cytometry analysis of antigen uptake. Schematic shows gating strategy to identify AF647-labeled vaccine uptake in macrophages, B cells, and dendritic cells of the NALT. MEW, major histocompatibility complex.

FIGS. 11A-11E show GC B cell responses in mouse NALT following intranasal immunization with amph-protein. Groups of C57Bl/6 (WT) or FcRn−/− mice (n=5 animals/group) were immunized with 5 μg eOD or amph-eOD mixed with 5 μg SMNP adjuvant, and GC responses were analyzed by flow cytometry on day 12. FIG. 11A: Gating strategy for identification of GC B cells. FIG. 11B: Representative FACS plots and FIG. 11C: absolute number of cells showing total CD38-GL7+GC B cells for all NALT samples, including controls. FIG. 11D: Representative FACS plots and FIG. 11E: absolute number of cells showing eOD-tetramer+ GC B cells for all NALT samples, including controls. Statistical significance determined using one-way ANOVA followed by Tukey's post-hoc test (*p<0.05, **p<0.01, ***p<0.001, ****p<0.0001). All data showing mean±s.e.m.

FIGS. 12A-12E show Tfh cell responses in mouse NALT following intranasal immunization with amph-protein. Groups of C57Bl/6 (WT) or FcRn−/− mice (n=5 animals/group) were immunized with 5 μg eOD or amph-eOD mixed with 5 μg SMNP adjuvant, and TFH responses were analyzed by flow cytometry on day 12. FIG. 12A: Gating strategy for identification of Tfh cells. (FIG. 12B) Representative FACS plots and (FIG. 12C) absolute number of cells showing activated ICOS+CD4+CD44+ T cells for all NALT samples, including controls. (FIG. 12D) Representative FACS plots and (FIG. 12E) absolute number of cells showing PD-1+CXCR5+ Tfh cells for all NALT samples, including controls. Statistical significance determined using one-way ANOVA followed by Tukey's post-hoc test (*p<0.05, **p<0.01). All data showing mean±s.e.m.

FIGS. 13A-13C show control parenteral immunization with amph-protein conjugate elicits negligible mucosal antibody response compared to intranasal immunization. BALB/c mice (n=5 animals per group) were immunized intranasally (i.n.) or subcutaneously (s.c.) injected at the scruff with 5 μg amph-eOD mixed with 25 μg cdGMP adjuvant and boosted 6 weeks later with the same formulation (arrows). IgG and IgA titers were measured in the (FIG. 13A) serum, (FIG. 13B) vaginal wash, and (FIG. 13C) feces. Statistical significance comparing i.n. and s.c. groups was determined by unpaired t-test. *p<0.05, **p<0.01, ***p<0.001, ****p<0.0001. All data are presented as mean±s.e.m.

FIGS. 14A-14C show that long-lived antigen-specific IgG and IgA plasma cells established in mice following intranasal immunization with amph-protein, without induction of anti-PEG antibodies. BALB/c mice (n=3 animals/group) were immunized i.n. with 5 μg amph-eOD mixed with 25 μg cdGMP adjuvant and boosted 6 weeks later with the same formulation. Female reproductive tract (FRT) and bone marrow (BM) eOD specific IgG and IgA antibody-secreting cells were assessed by ELISPOT at 20 wks post immunization: (A) representative well images and (B) quantified number of antibody-secreting plasma cells per 500,000 cells. All data showing mean±s.e.m. FIG. 14C: Serum samples from mice immunized as in FIGS. 4A and F with saponin (collected at week 11) or cdGMP adjuvants (collected at week 12) were analyzed by ELISA for anti-PEG IgG, comparing to a reference anti-PEG IgG standard.

FIGS. 15A-15F show synthesis and characterization of amph-RBD. FIG. 15A: Gel of RBD versus cys-RBD. FIG. 15B: Antigenicity ELISA results comparing binding of RBD versus cys-RBD to monoclonal antibodies CR3022 and angiotensin converting enzyme 2 (ACE2)-Fc. FIG. 15C: Dynamic light scattering analysis of RBD and amph-RBD. is shown as number-weighted % frequency. Dₕ, hydrodynamic diameter. FIG. 15D: The size exclusion chromatography (SEC) profile of RBD versus amph-RBD. FIGS. 15E-15F: ACE2 binding inhibition raw absorbance curves for week 6 serum (FIG. 15E) and bronchoalveolar lavage fluid (BALF) (FIG. 15F), used to determine IC50 values shown in FIG. 5E. All data showing mean±s.e.m.

FIGS. 16A-16D show that intranasal immunization with amph-protein conjugates leads to improved humoral immune responses in non-human primates. Rhesus macaques (n=6 animals/group) were immunized i.n. with 100 μg eOD or amph-eOD mixed with 375 μg SMNP adjuvant and boosted at 8, 16, and 24 wks with the same formulations (arrows in FIGS. 16C and 16D). FIG. 16A: Percent antigen-specific IgM, IgG, and IgA secreting plasma blasts in peripheral blood as determined by ELISPOT (% eOD of total); FIG. 16B: total IgM, IgG, and IgA secreting plasma blasts frequencies; FIG. 16C: vaginal IgG and IgA titers over time; FIG. 16D: rectal IgG and IgA titers over time. Statistical significance for FIGS. 16A-16B was determined using multiple unpaired t-tests. Statistical significance for FIGS. 16C-16D was determined using a two-way ANOVA comparing eOD and amph-eOD across all timepoints. (*p<0.05, **p<0.01, ***p<0.001, ****p<0.0001). All data showing mean±s.e.m.

FIGS. 17A-17B show synthesis of amphiphile MD39 conjugates. FIG. 17A: Reduced MD39 trimer protein with terminal cysteine (cys-MD39) was first reacted with a linker, DBCO-PEG4-maleimide, to form intermediate product DBCO-PEG4-MD39. FIG. 17B: DBCO-PEG4-MD39 was then reacted with DSPE-PEG2K-azide in a click chemistry reaction to form final product amph-MD39, which may exist as amph-MD39 monomer conjugates or amph-MD39 trimer conjugates.

FIG. 18 shows characterization of amph-MD39 by UV-Vis spectrophotometry. MD39 protein with terminal cysteine (cys-MD39, spectra shown in solid gray line) was first reacted with a linker, DBCO-PEG4-maleimide, to form intermediate product DBCO-PEG4-MD39 (‘amph pre click’, spectra shown in solid black line), identified with the presence of a DBCO peak at 309 nm. DBCO-PEG4-MD39 was then reacted with DSPE-PEG2K-azide in a click chemistry reaction to form final product amph-MD39 (‘amph post click’, spectra shown in dotted line). The absence of a DBCO peak at 309 nm in the final product provided evidence that the reaction progressed to completion. MD39 protein was identified and quantified using the peak at 280 nm. The concentration of MD39 in amph-MD39 product was quantified using the peak at 280 nm corrected for the background lipid absorbance from 310-500 nm.

FIGS. 19A-19B show that amph-MD39 trimer conjugates elicited enhanced systemic and mucosal immune responses after intranasal immunization. FIG. 19A: BALB/c mice (n=5 animals per group) were immunized intranasally with 5 μg of MD39 or amph-MD39 mixed with 5 μg of saponin MPLA nanoparticle (SMNP) adjuvant and boosted at 6 and 12 weeks with the same formulations. FIG. 19B: Antigen-specific serum IgG and vaginal mucosal IgA titers were measured by ELISA against MD39. Red arrows indicate vaccination. Statistical significance was determined by ordinary two-way ANOVA followed by Sidak's post hoc test, comparing MD39 to amph-MD39 at each time point. *p<0.05, **p<0.01, ***p<0.001, and ****p<0.0001. All data show means±SD.

### DETAILED DESCRIPTION

### Overview

Humoral immune (antibody) response is desired both systemically and at localized mucosal surfaces to combat infectious pathogens that infect a host through mucosal transmission.

The present inventions are based on the surprising findings that vaccines comprising large protein antigens conjugated to a lipid tail (amphiphilic conjugates) can elicit humoral immune responses to the antigens, such as for example HIV and SARS-CoV-2, significantly more effectively than free protein antigens after transmucosal (e.g., intranasal) administration. Amphiphilic conjugates comprising protein antigens surprisingly showed enhanced persistence and uptake across the mucosa compared to unmodified antigens, leading to greatly increased germinal center (GC) and follicular helper T cell (Tfh) responses in the nasal associated lymphoid tissue (NALT). Intranasal (i.n.) immunization with the amphiphilic conjugates also surprisingly led to high levels of IgG and IgA in serum, upper and lower respiratory mucosa, and distal genitourinary mucosal sites, including the induction of substantial neutralizing antibody responses in mice. Further, intranasal immunization with the amphiphilic conjugates enhanced vaccine uptake in the nasal passages and enhanced IgG and IgA responses relative to soluble protein immunization in non-human primates.

Thus, the present disclosure provides vaccines suitable for transmucosal administration, and methods of use thereof to induce an immune response or immunity (e.g., involving a humoral antibody response) against an infections pathogen.

### Definitions

Terms used in the claims and specification are defined as set forth below unless otherwise specified.

It must be noted that, as used in the specification and the appended claims, the singular forms “a,” “an” and “the” include plural referents unless the context clearly dictates otherwise.

As used herein, “about” will be understood by persons of ordinary skill and will vary to some extent depending on the context in which it is used. If there are uses of the term which are not clear to persons of ordinary skill given the context in which it is used, “about” will mean up to plus or minus 10% of the particular value.

As used herein, the term “adjuvant” refers to a compound that, with a specific immunogen or antigen, will augment or otherwise alter or modify the resultant immune response. Modification of the immune response includes intensification or broadening the specificity of either or both antibody and cellular immune responses. Modification of the immune response can also mean decreasing or suppressing certain antigen-specific immune responses. In certain embodiments, the adjuvant is a cyclic dinucleotide. In some embodiments, the adjuvant is an immunostimulatory oligonucleotide as described herein. In some embodiments, the adjuvant is administered prior to, concurrently, or after administration of an amphiphilic conjugate, or composition comprising the conjugate. In some embodiments, the adjuvant is co-formulated in the same composition as an amphiphilic conjugate.

“Amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, y-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid.

Amino acids can be referred to herein by either their commonly known three letter symbols or by the one-letter symbols recommended by the IUPAC-IUB Biochemical Nomenclature Commission. Nucleotides, likewise, can be referred to by their commonly accepted single-letter codes.

An “amino acid substitution” refers to the replacement of at least one existing amino acid residue in a predetermined amino acid sequence (an amino acid sequence of a starting polypeptide) with a second, different “replacement” amino acid residue. An “amino acid insertion” refers to the incorporation of at least one additional amino acid into a predetermined amino acid sequence. While the insertion will usually consist of the insertion of one or two amino acid residues, the present larger “peptide insertions,” can be made, e.g. insertion of about three to about five or even up to about ten, fifteen, or twenty amino acid residues. The inserted residue(s) may be naturally occurring or non-naturally occurring as disclosed above. An “amino acid deletion” refers to the removal of at least one amino acid residue from a predetermined amino acid sequence.

As used herein, “amphiphile” or “amphiphilic” refers to a conjugate comprising a hydrophilic head group and a hydrophobic tail, thereby forming an amphiphilic conjugate. In some embodiments, an amphiphilic conjugate comprises an immunogen, e.g., a protein antigen, and one or more hydrophobic lipid tails. In some embodiments, the amphiphile conjugate further comprises a polymer (e.g., polyethylene glycol), wherein the polymer is conjugated to the one or more lipids and/or the immunogen.

The term “ameliorating” refers to any therapeutically beneficial result in the treatment of a disease state, e.g., cancer, including prophylaxis, lessening in the severity or progression, remission, or cure thereof.

As used herein, the term “antibody” refers to an immunoglobulin molecule comprising four polypeptide chains, two heavy chains (HC) and two light chains (LC) inter- connected by disulfide bonds. An antibody consists of two structural regions: a variable fragment (Fab) that mediates antigen binding and a constant fragment (Fc) that mediates downstream effector functions.

There are five immunoglobulin classes (isotypes) of antibody molecules found in serum: IgG, IgM, IgA, IgE, and IgD. They are distinguished by the type of heavy chain they contain. IgG molecules possess heavy chains known as γ-chains; IgMs have μ-chains; IgAs have α-chains; IgEs have c-chains; and IgDs have δ-chains. The variation in heavy chain polypeptides allows each immunoglobulin class to function in a different type of immune response or during a different stage of the body's defense. The amino acid sequences that confer these functional differences are located mainly within the Fc domain. IgG (immunoglobulin G) is expressed on the surface of mature B cells, and is also the most prevalent Ig in serum and extravascular spaces. IgG has 4 subtypes: IgG1, IgG2, IgG3 and IgG4. IgA (immunoglobulin A) plays a pivotal role in mucosal homeostasis in the gastrointestinal, respiratory, and genitourinary tracts, functioning as the dominant antibody of immunity in this role. IgA has two subtypes: IgA1 and IgA2.

Immunoglobulin class switching, also known as isotype switching, is a biological mechanism that changes a B cell's production of immunoglobulin from one type to another. Class switching occurs rapidly after activation of mature naïve B cells, resulting in a switch from expressing IgM and IgD to expression of IgG, IgE, or IgA; this switch improves the ability of antibodies to remove the pathogen that induces the humoral immune response.

As used herein, the terms “antigen” or “immunogen” refer to molecule which, when administered to a vertebrate, especially a mammal, will induce an immune response.

The terms “antigenic peptide” or “peptide antigen”, used interchangeably herein, refer to a peptide which, when administered to a vertebrate, especially a mammal, will induce an immune response, e.g., a cell-mediated immune response.

The terms “antigenic protein” or “protein antigen”, as used herein, refer to a protein which, when administered to a vertebrate, especially a mammal, will induce an immune response, e.g., a humoral antibody mediated immune response.

The term “antigen presenting cell” or “APC” is a cell that displays foreign antigen complexed with MHC on its surface. T cells recognize this complex using T cell receptor (TCR). Examples of APCs include, but are not limited to, dendritic cells (DCs), peripheral blood mononuclear cells (PBMC), monocytes (such as THP-1), B lymphoblastoid cells (such as C1R.A2, 1518 B-LCL) and monocyte-derived dendritic cells (DCs). Some APCs internalize antigens either by phagocytosis or by receptor-mediated endocytosis.

The term “B cells” refers to a type of lymphocytes that are responsible for mediating the production of antigen-specific immunoglobulin (Ig) directed against invasive pathogens that are typically known as antibodies.

As used herein, “CG oligodeoxynucleotides (CG ODNs)”, also referred to as “CpG ODNs”, are short single-stranded synthetic DNA molecules that contain a cytosine nucleotide (C) followed by a guanine nucleotide (G). In certain embodiments, the immunostimulatory oligonucleotide is a CG ODN.

A polypeptide or amino acid sequence “derived from” a designated polypeptide or protein refers to the origin of the polypeptide. Preferably, the polypeptide or amino acid sequence which is derived from a particular sequence has an amino acid sequence that is essentially identical to that sequence or a portion thereof, wherein the portion consists of at least 10-20 amino acids, preferably at least 20-30 amino acids, more preferably at least 30-50 amino acids, or which is otherwise identifiable to one of ordinary skill in the art as having its origin in the sequence.

Polypeptides derived from another peptide may have one or more mutations relative to the starting polypeptide, e.g., one or more amino acid residues which have been substituted with another amino acid residue or which has one or more amino acid residue insertions or deletions.

A polypeptide can comprise an amino acid sequence which is not naturally occurring. Such variants necessarily have less than 100% sequence identity or similarity with the starting molecule. In a preferred embodiment, the variant will have an amino acid sequence from about 75% to less than 100% amino acid sequence identity or similarity with the amino acid sequence of the starting polypeptide, more preferably from about 80% to less than 100%, more preferably from about 85% to less than 100%, more preferably from about 90% to less than 100% (e.g., 91%, 92%, 93%, 94%, 95%, 96%, 97%, 98%, 99%) and most preferably from about 95% to less than 100%, e.g., over the length of the variant molecule.

In one embodiment, there is one amino acid difference between a starting polypeptide sequence and the sequence derived therefrom. Identity or similarity with respect to this sequence is defined herein as the percentage of amino acid residues in the candidate sequence that are identical (i.e., same residue) with the starting amino acid residues, after aligning the sequences and introducing gaps, if necessary, to achieve the maximum percent sequence identity.

As used herein, the term antigen “cross-presentation” refers to presentation of exogenous protein antigens to T cells via MEW class I and class II molecules on APCs.

As used herein, the term “cytotoxic T lymphocyte (CTL) response” refers to an immune response induced by cytotoxic T cells. CTL responses are mediated primarily by CD8+ T cells.

As used herein, the term “effective amount” or “effective dose” is defined as an amount sufficient to achieve or at least partially achieve the desired effect, such as e.g., inducing or enhancing an immune response, or providing immunity, to an immunogen. The term “therapeutically effective amount” or “therapeutically effective dose” is defined as an amount that is effective to ameliorate a symptom of a disease. A therapeutically effective amount can be “prophylactically effective amount” as prophylaxis can be considered therapy.

As used herein, the term “effector cell” or “effector immune cell” refers to a cell involved in an immune response, e.g., in the promotion of an immune effector response. In some embodiments, immune effector cells specifically recognize an antigen. Examples of immune effector cells include, but are not limited to, Natural Killer (NK) cells, B cells, monocytes, macrophages, T cells (e.g., cytotoxic T lymphocytes (CTLs)). In some embodiments, the effector cell is a T cell.

As used herein the term “humoral immune response” is an immune response mediated by antibody molecules that are secreted by B cells. The presence of antigens triggers B cell activation and differentiation into antibody-secreting plasma cells and usually requires helper T cells (which are CD4+ T cells).

As used herein, the term “immune effector function” or “immune effector response” refers to a function or response of an immune effector cell that promotes an immune response to a target.

As used herein, “immune cell” is a cell of hematopoietic origin and that plays a role in the immune response. Immune cells include lymphocytes (e.g., B cells and T cells), natural killer cells, and myeloid cells (e.g., monocytes, macrophages, eosinophils, mast cells, basophils, and granulocytes).

As used herein, an “immunostimulatory oligonucleotide” is an oligonucleotide that can stimulate (e.g., induce or enhance) an immune response.

The terms “inducing an immune response” and “enhancing an immune response” are used interchangeably and refer to the stimulation of an immune response (i.e., either passive or adaptive) to a particular antigen. The term “induce” as used with respect to inducing CDC or ADCC refer to the stimulation of particular direct cell killing mechanisms.

As used herein, a subject “in need of prevention,” “in need of treatment,” “in need of immunization”, or “in need thereof,” refers to one, who by the judgment of an appropriate medical practitioner (e.g., a doctor, a nurse, or a nurse practitioner in the case of humans; a veterinarian in the case of non-human mammals), would reasonably benefit from a given treatment (such as treatment with a composition comprising an amphiphilic conjugate for immunization against an immunogen).

As used herein, “intranasal administration” refers to a route of transmucosal drug administration wherein a drug (e.g., vaccine) is insufflated through the nose, and enters through or across nasal mucosal epithelium to underlying cells/tissue. In embodiments, intranasal administration provides local delivery, systemic delivery, or both local and systemic delivery of the drug.

The term “in vivo” refers to processes that occur in a living organism.

As used herein, the terms “linked”, “operably linked,” “fused”, or “fusion”, are used interchangeably. These terms refer to the joining together of two more elements or components or domains, by an appropriate means including chemical conjugation or recombinant DNA technology. Methods of chemical conjugation (e.g., using heterobifunctional crosslinking agents or using “click” chemistry) are known in the art as are methods of recombinant DNA technology.

The term “lipid” refers to a biomolecule that is soluble in nonpolar solvents and insoluble in water. Lipids are often described as hydrophobic or amphiphilic molecules which allows them to form structures such as vesicles or membranes in aqueous environments. Lipids include fatty acids, glycerolipids, glycerophospholipids, sphingolipids, sterol lipids (including cholesterol), prenol lipids, saccharolipids, and polyketides. In some embodiments, the lipid suitable for the amphiphilic conjugates of the disclosure binds to human serum albumin under physiological conditions. In some embodiments, the lipid suitable for the amphiphilic conjugates of the disclosure inserts into a cell membrane under physiological conditions. In some embodiments, the lipid binds albumin and inserts into a cell membrane under physiological conditions. In some embodiments, the lipid is a diacyl lipid. In some embodiments, the diacyl lipid comprises more than 12 carbons. In some embodiments, the diacyl lipid comprises at least 13, at least 14, at least 15, at least 16, at least 17 or at least 18 carbons.

As used herein, “neutralizing antibody” refers to an antibody that not only binds to a pathogen (e.g., a virus, a bacteria) but also binds in a manner that prevents infection. For example, a neutralizing antibody may block interaction of a viral capsid protein with a receptor on a host cell, thereby preventing the virus from entering a host cell. Only a small subset of antibodies that bind a pathogen are capable of neutralization. After an infection, it can take some time for a subject to produce highly effective neutralizing antibodies, but these can persist to protect against future encounters with the agent.

“Nucleic acid” refers to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences and as well as the sequence explicitly indicated. Specifically, degenerate codon substitutions can be achieved by generating sequences in which the third position of one or more selected (or all) codons is substituted with mixed base and/or deoxyinosine residues (Batzer et al., Nucleic Acid Res. 19:5081, 1991; Ohtsuka et al., J. Biol. Chem. 260:2605-2608, 1985); and Cassol et al., 1992; Rossolini et al., Mol. Cell. Probes 8:91-98, 1994). For arginine and leucine, modifications at the second base can also be conservative. The term nucleic acid is used interchangeably with gene, cDNA, and mRNA encoded by a gene.

In some embodiments, the peptides of the invention are encoded by a nucleotide sequence. Nucleotide sequences of the invention can be useful for a number of applications, including: cloning, gene therapy, protein expression and purification, mutation introduction, DNA vaccination of a host in need thereof, antibody generation for, e.g., passive immunization, PCR, primer and probe generation, and the like.

As used herein, “parenteral administration,” “administered parenterally,” and other grammatically equivalent phrases, refer to modes of administration other than enteral and topical administration, usually by injection, and include, without limitation, intravenous, intranasal, intraocular, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural, intracerebral, intracranial, intracarotid and intrasternal injection and infusion.

As generally used herein, “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

As used herein, the term “physiological conditions” refers to the in vivo condition of a subject. In some embodiments, physiological condition refers to a neutral pH (e.g., pH between 6-8).

“Polypeptide,” “peptide”, and “protein” refer to a polymer of amino acid residues. The terms apply to naturally occurring amino acid polymers and non-naturally occurring amino acid polymer amino acid polymers, including in which one or more amino acid residue is an artificial chemical mimetic of a corresponding naturally occurring amino acid.

As used herein, “protein” refers to a molecule that comprises or consists of more than 50 amino acids. As used herein, “peptide” refers to a molecule that consists of between 2 and 50 amino acids. An “oligopeptide” refers to a molecule that consists of between 2 and about 20 amino acids.

As used herein, a “small molecule” is a molecule with a molecular weight below about 500 Daltons.

As used herein, the term “subject” includes any human or non-human animal. The term “non-human animal” includes all vertebrates, e.g., mammals and non-mammals, such as non-human primates, canines, felines, murines, bovines, equines, porcines, sheep, chickens, amphibians, or reptiles.

The term “sufficient amount” or “amount sufficient to” means an amount sufficient to produce a desired effect, e.g., an amount sufficient to immunize a subject against an immunogen.

The term “T cell” refers to a type of white blood cell that can be distinguished from other white blood cells by the presence of a T cell receptor on the cell surface. There are several subsets of T cells, including, but not limited to, T helper cells (a.k.a. TH cells or CD4+ T cells) and subtypes, including TH1, TH2, TH3, TH17, TH9, and TFH cells, cytotoxic T cells (i.e., TC cells, CD8+ T cells, cytotoxic T lymphocytes, T-killer cells, killer T cells), memory T cells and subtypes, including central memory T cells (TCM cells), effector memory T cells (TEM and TEMRA cells), and resident memory T cells (TRM cells), regulatory T cells (a.k.a. Treg cells or suppressor T cells) and subtypes, including CD4+FOXP3+ Treg cells, CD4+FOXP3− Treg cells, Tr1 cells, Th3 cells, and Treg17 cells, natural killer T cells (a.k.a. NKT cells), mucosal associated invariant T cells (MAITs), and gamma delta T cells (γδ T cells), including Vγ9/Vδ2 T cells. Any one or more of the aforementioned or unmentioned T cells may be the target cell type for a method of use of the invention.

As used herein, the term “T cell activation” or “activation of T cells” refers to a cellular process in which mature T cells, which express antigen-specific T cell receptors on their surfaces, recognize their cognate antigens and respond by entering the cell cycle, secreting cytokines or lytic enzymes, and initiating or becoming competent to perform cell-based effector functions. T cell activation requires at least two signals to become fully activated. The first occurs after engagement of the T cell antigen-specific receptor (TCR) by the antigen-major histocompatibility complex (MEW), and the second by subsequent engagement of co-stimulatory molecules (e.g., CD28). These signals are transmitted to the nucleus and result in clonal expansion of T cells, upregulation of activation markers on the cell surface, differentiation into effector cells, induction of cytotoxicity or cytokine secretion, induction of apoptosis, or a combination thereof.

As used herein, the term “T cell-mediated response” refers to any response mediated by T cells, including, but not limited to, effector T cells (e.g., CD8+ cells) and helper T cells (e.g., CD4+ cells). T cell mediated responses include, for example, T cell cytotoxicity and proliferation.

The term “T cell cytotoxicity” includes any immune response that is mediated by CD8+ T cell activation. Exemplary immune responses include cytokine production, CD8+ T cell proliferation, granzyme or perforin production, and clearance of an infectious agent.

As used herein, “transmucosal administration” refers to a route of drug administration wherein a drug (e.g., vaccine) enters through or across a mucosal epithelium to underlying tissue. In some embodiments, a drug administered transmucosally enters systemic circulation. In embodiments, transmucosal administration provides local delivery of the drug. In some embodiments, transmucosal administration provides both local and systemic delivery of the drug.

The terms “treat,” “treating,” and “treatment,” as used herein, refer to therapeutic or preventative measures described herein. The methods of “treatment” employ administration to a subject in need of such treatment a vaccine or amphiphilic conjugate of the present disclosure, for example, a subject at risk of infection with an immunogen. In some embodiments, an amphiphilic conjugate is administered to a subject in need of an enhanced immune response against a particular antigen or a subject who ultimately may acquire such a disorder, in order to prevent, cure, delay, reduce the severity of, or ameliorate one or more symptoms of the disorder or recurring disorder.

As used herein, “vaccine” refers to a composition which contains an amphiphilic conjugate as described herein, which is in a form that is capable of being administered (e.g., transmucosally or intranasally) to a subject, and which is capable of inducing a protective immune response. In embodiments, the protective immune response is sufficient to induce immunity, and/or to prevent and/or ameliorate an infection or disease, and/or to reduce at least one symptom of an infection or disease, and/or to enhance the efficacy of another dose of the amphiphilic conjugate. Upon introduction into a host, the vaccine provokes an immune response including, but not limited to, the production of antibodies and/or cytokines and/or the activation of cytotoxic T cells, antigen presenting cells, helper T cells, dendritic cells and/or other cellular responses.

### Amphiphilic Conjugates

In some aspects, the present disclosure provides a vaccine comprising an immunogen (e.g., peptide antigen or protein antigen) operably linked to an albumin-binding lipid, wherein the vaccine is suitable for transmucosal administration (e.g., nasal administration) to induce an immune response (e.g., a cell-mediated immune response or a humoral antibody-mediated immune response).

An amphiphile vaccine technology has been previously developed that involves linking adjuvants or antigenic peptides to lipophilic polymeric tails, which promotes localization of vaccines to lymph node (Liu et al. (2014) Nature 507:519-522). Such amph-peptides are also capable of inserting into cell membranes (see e.g., Liu et al. (2011) Angewandte Chemie-Intl. Ed. 50:7052-7055). Prior studies of amphiphile vaccines, however, focused on amphiphilic conjugates comprising relatively low molar mass peptide antigens targeting T cell immunity, and that were systemically introduced into a subject via intravenous or subcutanenous injection. The present disclosure provides amphiphilic conjugates comprising immunogens, such as peptide antigens or protein antigens, that are suitable for transmucosal delivery (e.g., intranasal administration) and which stimulate a protective immune response, locally and/or systemically.

A diversity of amphiphilic conjugate structures are provided, wherein a lipophilic albumin-binding moiety, or “lipid tail” (e.g. DSPE), is linked (e.g., covalently linked) via a linker (e.g., PEG linker) to an immunogen, such as a peptide or protein antigen. Without being bound by theory, the amphiphilic conjugate comprised in the vaccine of the disclosure is believed to use albumin as a noncovalent chaperone to traverse across mucosal surfaces through interactions of albumin with the neonatal Fc receptor (FcRn) expressed by mucosal epithelial cells. Increased uptake of the amphiphilic conjugate across mucus and ephithelial lining results in enhanced immune responses in local tissues, e.g., lymphoid tissues.

In some embodiments, an amphiphilic conjugate comprised in a vaccine of the present disclosure is a lipid conjugate as described in US 2013/0295129, the entire contents of which are incorporated herein by reference. In some embodiments, an amphiphilic conjugate comprises a hydrophobic tail that inserts into a cell membrane. In some embodiments, the hydrophobic tail enhances association of the conjugate to cell surfaces (e.g., epithelial cell surfaces). In some embodiments, the hydrophobic tail enables the amphiphilic conjugate to tether to cell membrane and retain the amphiphilic conjugate at a localized tissue (e.g., mucosal epithelium). In some embodiments, the hydrophobic tail enables the amphiphilic conjugate to tether to cell membrane and retain the amphiphilic conjugate at a tissue near the site of administration. In some embodiments, the hydrophobic tail enables the amphiphilic conjugate to tether to cell membrane and reduces systemic circulation of the conjugate. In some embodiments, the hydrophobic tail enables the amphiphilic conjugate to tether to cell membrane and reduces distribution of the conjugate to distal tissues.

In some embodiments, an amphiphilic conjugate of the present disclosure comprises an albumin-binding lipid, wherein the albuin-binding lipid allows the conjugate to efficiently cross a mucosal epithelium with albumin in vivo. In some embodiments, the amphiphilic conjugate comprises an albumin-binding lipid comprising a hydrophobic tail, wherein the hydrophobic tail inserts into the cell membrane, and wherein the conjugate efficiently crosses a mucosal epithelium with albumin in vivo. In some embodiments, the amphiphilic conjugate binds to endogenous albumin, which enables uptake of the conjugate with albumin by the neonatal Fc receptor (FcRn) and targets the conjugate to local lymphoid tissues where it accumulates. In some embodiments, the amphiphilic conjugate includes an immunogen, such as an antigenic peptide or protein antigen, and thereby induces or enhances a protective immune response.

In some embodiments, the amphiphilic conjugates are efficiently targeted to the lymph nodes or local lymphoid-tissue. In some embodiments, the lymph node-targeting conjugates comprise a highly lipophilic, albumin-binding domain (e.g., an albumin-binding lipid), and a cargo such as an immunogen (e.g., antigenic peptide, or protein antigen). In some embodiments, lymph node-targeting conjugates include three domains: a highly lipophilic, albumin-binding domain (e.g., an albumin-binding lipid), a cargo such as an immunogen (e.g., antigenic peptide or protein antigen), and a linker (e.g., a polar block linker) which promotes solubility of the conjugate. Accordingly, in certain embodiments, the general structure of the amphiphilic conjugate is L-P-C, where “L” is an albumin-binding lipid, “P” is a polar block linker, and “C” is a cargo such as an immunogen (e.g., antigenic peptide or protein antigen). In some embodiments, the cargo itself can also serve as the polar block domain, and a separate polar block domain is not required. Therefore, in certain embodiments the conjugate has only two domains: an albumin-binding lipid and a cargo such as an immunogen (e.g., antigenic peptide or protein antigen).

In some embodiments, the amphiphilic conjugate is administered or formulated with an adjuvant.

### (i) Lipids

In some embodiments, the lipid component of the amphiphilic conjugates of the present disclosure comprises a hydrophobic tail. In some embodiments, the hydrophobic tail inserts or is capable of inserting into a cell membrane. In some embodiments, the lipid is linear, branched, or cyclic. In some embodiments, the lipid is greater than 12 carbons in length. In some embodiments, the lipid is 13 carbons in length. In some embodiments, the lipid is 14 carbons in length. In some embodiments, the lipid is 15 carbons in length. In some embodiments, the lipid is 16 carbons in length. In some embodiments, the lipid is 17 carbons in length. In some embodiments, the lipid is 18 carbons in length. In some embodiments, the lipid is 19 carbons in length. In some embodiments, the lipid is 20 carbons in length. In some embodiments, the lipid is 21 carbons in length. In some embodiments, the lipid is 22 carbons in length. In some embodiments, the lipid is 23 carbons in length. In some embodiments, the lipid is 24 carbons in length. In some embodiments, the lipid is 25 carbons in length. In some embodiments, the lipid is 26 carbons in length. In some embodiments, the lipid is 27 carbons in length. In some embodiments, the lipid is 28 carbons in length. In some embodiments, the lipid is 29 carbons in length. In some embodiments, the lipid is 30 carbons in length. In some embodiments, the lipid at least 17 to 18 carbons in length, but may be shorter if it shows good albumin binding and adequate targeting to the lymph nodes.

In certain embodiments, the activity of the amphiphilic conjugate relies, in part, on the ability of the conjugate to target the lymph nodes. In certain embodiments, the activity of the amphiphilic conjugate relies, in part, on the ability of the conjugate to associate with albumin, e.g., in the blood, tissues, lymph, or mucosal epithelium, of the subject. In some embodiments, the activity of the amphiphilic conjugate relies, in part, on the ability of the conjugate to associate with albumin and be transported across mucosal barriers via interaction of albumin with the FcRn expressed by mucosal epithelial cells. Therefore, amphiphilic conjugates of the present disclosure typically include a lipid that can bind to albumin. In preferred embodiments, the amphiphilic conjugates include a lipid that can bind to albumin under physiological conditions.

Lipids suitable for targeting the lymph node and/or for transporting the conjugate across mucosal epithelium can be selected based on the ability of the lipid or a lipid conjugate including the lipid to bind to albumin. Suitable methods for testing the ability of the lipid or lipid conjugate to bind to albumin are known in the art. For example, in certain embodiments, a plurality of lipid conjugates is allowed to spontaneously form micelles in aqueous solution. The micelles are incubated with albumin, or a solution including albumin such as Fetal Bovine Serum (FBS). Samples can be analyzed, for example, by ELISA, size exclusion chromatography or other methods to determine if binding has occurred. Lipid conjugates can be selected as lymph node-targeting conjugates if in the presence of albumin, or a solution including albumin such as Fetal Bovine Serum (FBS), the micelles dissociate and the lipid conjugates bind to albumin as discussed above.

Examples of preferred lipids for use in lymph node targeting lipid conjugates include, but are not limited to, fatty acids with aliphatic tails of 8-30 carbons including, but not limited to, linear unsaturated and saturated fatty acids, branched saturated and unsaturated fatty acids, and fatty acids derivatives, such as fatty acid esters, fatty acid amides, and fatty acid thioesters, diacyl lipids, cholesterol, cholesterol derivatives, and steroid acids such as bile acids, Lipid A or combinations thereof. In some embodiments, the lipid is saturated. In some embodiments, the lipid comprises at least one lipid tail comprising 8-30, 12-30, 15-25, or 16-20 carbons.

In certain embodiments, the lipid is a diacyl lipid or two-tailed lipid. In some embodiments, the tails in the diacyl lipid contain from about 8 to about 30 carbons and can be saturated, unsaturated, or combinations thereof. In some embodiments, the diacyl lipid is saturated. In some embodiments, the diacyl lipid is saturated and each tail comprises about 8 to about 30 carbons. In some embodiments, the diacyl lipid is saturated and each tail comprises 12 carbons. In some embodiments, the diacyl lipid is saturated and each tail comprises 13 carbons. In some embodiments, the diacyl lipid is saturated and each tail comprises 14 carbons. In some embodiments, the diacyl lipid is saturated and each tail comprises 15 carbons. In some embodiments, the diacyl lipid is saturated and each tail comprises 16 carbons. In some embodiments, the diacyl lipid is saturated and each tail comprises 17 carbons. In some embodiments, the diacyl lipid is saturated and each tail comprises 18 carbons. In some embodiments, the diacyl lipid is saturated and each tail comprises 19 carbons. In some embodiments, the diacyl lipid is saturated and each tail comprises 20 carbons. In some embodiments, the diacyl lipid is saturated and each tail comprises 21 carbons. In some embodiments, the diacyl lipid is saturated and each tail comprises 22 carbons. In some embodiments, the diacyl lipid is saturated and each tail comprises 23 carbons. In some embodiments, the diacyl lipid is saturated and each tail comprises 24 carbons. In some embodiments, the diacyl lipid is saturated and each tail comprises 25 carbons. In some embodiments, the diacyl lipid is saturated and each tail comprises 26 carbons. In some embodiments, the diacyl lipid is saturated and each tail comprises 27 carbons. In some embodiments, the diacyl lipid is saturated and each tail comprises 28 carbons. In some embodiments, the diacyl lipid is saturated and each tail comprises 29 carbons. In some embodiments, the diacyl lipid is saturated and each tail comprises 30 carbons. The tails can be coupled to the head group via ester bond linkages, amide bond linkages, thioester bond linkages, or combinations thereof. In some embodiments, the diacyl lipids are phosphate lipids, glycolipids, sphingolipids, or combinations thereof.

In some embodiments, the lipid is 1,2-distearoyl-sn-glycero-3-phosphoethanolamine (DSPE). In some embodiments, a diacyl lipid is synthesized as described in U.S. Pat. No. 9,107,904, the entire contents of which are incorporated herein by reference. In some embodiments, a diacyl lipid is synthesized as provided below:

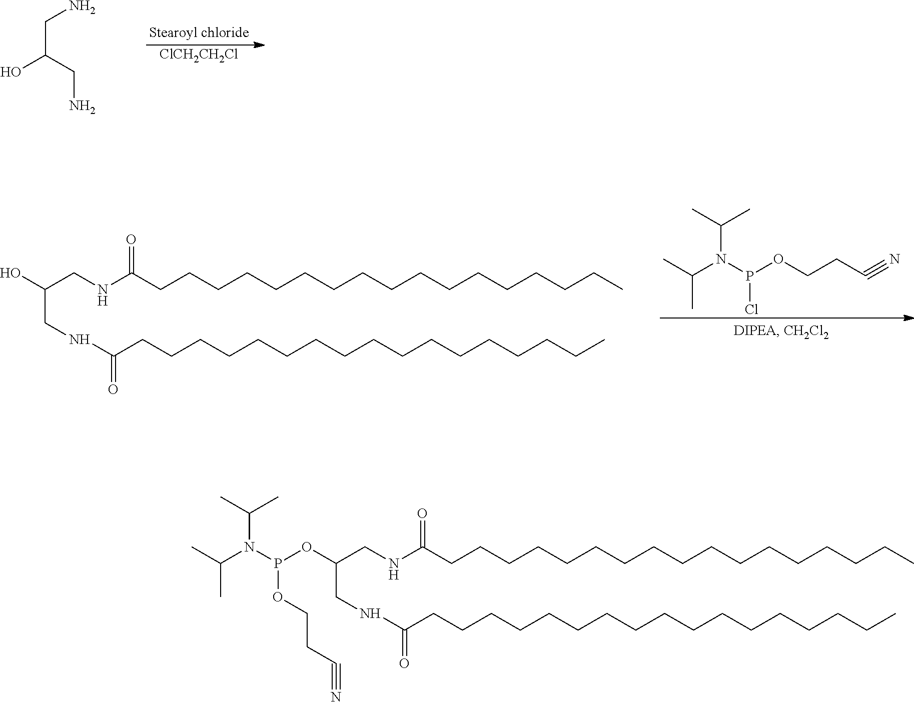

Preferably, amphiphilic conjugates include a lipid that is 8 or more carbon units in length. It is believed that increasing the number of lipid units can reduce insertion of the lipid into plasma membrane of cells, allowing the lipid conjugate to remain free to bind albumin and traffic across the mucosal epithelium and/or to the lymph node. For example, in some embodiments, the lipid can be a diacyl lipid composed of two C18 hydrocarbon tails. In some embodiments, the lipid for use in preparing amphiphilic conjugates is not a single chain hydrocarbon (e.g., C18).

### (ii) Cargo

In some aspects, the cargo of the amphiphilic conjugates provided herein is an immunogen. In some embodiments the immunogen is a peptide antigen (also referred to herein as an antigenic peptide), a protein antigen, or a polysaccharide antigen. In some embodiments, the immunogen is an antigenic peptide or a protein antigen. In some embodiments, the immunogen comprises or consists of an peptide antigen or a protein antigen. In some embodiments the immunogen comprises or consists of a polysaccharide antigen.

In some embodiments, the immunogen is an antigenic peptide. As used herein, an “antigenic peptide” has fewer than 50 amino acids and comprises at least one sequence of amino acids sufficient to elicit an immune response, e.g., cell-mediated immune response.

In some embodiments, the immunogen is not an antigenic peptide. In some embodiments, the immunogen does not elicit a cell-mediated immune response.

For many types of infectious diseases that transmit via muscosal routes such as HIV, SARS-CoV-2 and influenza, larger protein antigens that more closely resemble native proteins of the infectious agents are believed to be significantly more effective at inducing an immune response through vaccination than small peptides. Accordingly, in some embodiments, the immunogen is a protein antigen. As used herein, a “protein antigen” comprises at least 50 or more amino acids and includes at least one sequence of amino acids sufficient to elicit an immune response, e.g., a humoral antibody-mediated immune response.

In some embodiments, the protein antigen comprises at least 50 amino acids, at least 51 amino acids, at least 52 amino acids, at least 53 amino acids, at least 54 amino acids, at least 55 amino acids, at least 56 amino acids, at least 57 amino acids, at least 58 amino acids, at least 59 amino acids, at least 60 amino acids, at least 75 amino acids, at least 100 amino acids, at least 125 amino acids, at least 150 amino acids, at least 175 amino acids, at least 200 amino acids, at least 250 amino acids, at least 300 amino acids, at least 350 amino acids, at least 400 amino acids, at least 450 amino acids, at least 500 amino acids, at least 550 amino acids, at least 600 amino acids, at least 650 amino acids, at least 700 amino acids, at least 750 amino acids, at least 800 amino acids, at least 850 amino acids, at least 900 amino acids, at least 950 amino acids, at least 1000 amino acids, at least 1050 amino acids, at least 1100 amino acids, at least 1150 amino acids, at least 1200 amino acids, at least 1250 amino acids, at least 1300 amino acids, at least 1350 amino acids, at least 1400 amino acids, at least 1450 amino acids, at least 1500 amino acids, at least 1550 amino acids, at least 1600 amino acids, at least 1650 amino acids, at least 1700 amino acids, at least 1750 amino acids, at least 1800 amino acids, at least 1850 amino acids, at least 1900 amino acids, at least 1950 amino acids, at least 2000 amino acids, at least 2050 amino acids, at least 2100 amino acids, at least 2150 amino acids, at least 2200 amino acids, at least 2300 amino acids, at least 2400 amino acids, at least 2500 amino acids, at least 2600 amino acids, at least 2700 amino acids, at least 2800 amino acids, at least 2900 amino acids, at least 3000 amino acids, at least 3100 amino acids, at least 3200 amino acids, at least 3300 amino acids, at least 3400 amino acids, at least 3500 amino acids, at least 3600 amino acids, at least 3700 amino acids, at least 3800 amino acids, at least 3900 amino acids, at least 4000 amino acids, at least 4100 amino acids, at least 4200 amino acids, at least 4300 amino acids, at least 4400 amino acids, or at least 4500 amino acids.

In some embodiments, the protein antigen comprises greater than 50 amino acids, greater than 51 amino acids, greater than 52 amino acids, greater than 53 amino acids, greater than 54 amino acids, greater than 55 amino acids, greater than 56 amino acids, greater than 57 amino acids, greater than 58 amino acids, greater than 59 amino acids, greater than 60 amino acids, greater than 75 amino acids, greater than 100 amino acids, greater than 125 amino acids, greater than 150 amino acids, greater than 175 amino acids, greater than 200 amino acids, greater than 250 amino acids, greater than 300 amino acids, greater than 350 amino acids, greater than 400 amino acids, greater than 450 amino acids, greater than 500 amino acids, greater than 550 amino acids, greater than 600 amino acids, greater than 650 amino acids, greater than 700 amino acids, greater than 750 amino acids, greater than 800 amino acids, greater than 850 amino acids, greater than 900 amino acids, greater than 950 amino acids, greater than 1000 amino acids, greater than 1050 amino acids, greater than 1100 amino acids, greater than 1150 amino acids, greater than 1200 amino acids, greater than 1250 amino acids, greater than 1300 amino acids, greater than 1350 amino acids, greater than 1400 amino acids, greater than 1450 amino acids, greater than 1500 amino acids, greater than 1550 amino acids, greater than 1600 amino acids, greater than 1650 amino acids, greater than 1700 amino acids, greater than 1750 amino acids, greater than 1800 amino acids, greater than 1850 amino acids, greater than 1900 amino acids, greater than 1950 amino acids, greater than 2000 amino acids, greater than 2050 amino acids, greater than 2100 amino acids, greater than 2150 amino acids, greater than 2000 amino acids greater than 2300 amino acids, greater than 2400 amino acids, greater than 2500 amino acids, greater than 2600 amino acids, greater than 2700 amino acids, greater than 2800 amino acids, greater than 2900 amino acids, greater than 3000 amino acids, greater than 3100 amino acids, greater than 3200 amino acids, greater than 3300 amino acids, greater than 3400 amino acids, greater than 3500 amino acids, greater than 3600 amino acids, greater than 3700 amino acids, greater than 3800 amino acids, greater than 3900 amino acids, greater than 4000 amino acids, greater than 4100 amino acids, greater than 4200 amino acids, greater than 4300 amino acids, greater than 4400 amino acids, or greater than 4500 amino acids.

In some embodiments the protein antigen comprises about 50 to 5000 amino acids, about 50 to 4500 amino acids, about 50 to 4000 amino acids, about 50 to 3500 amino acids, about 50 to 3000 amino acids, or about 51 to 3000 amino acids. In some embodiments, the protein antigen comprises about 100 to 5000 amino acids, about 100 to 4500 amino acids, about 100 to 4000 amino acids, about 100 to 3500 amino acids, about 100 to 3000 amino acids, about 100 to about 2500 amino acids, about 100 to about 2000 amino acids, about 100 to about 1500 amino acids, about 100 to about 1000 amino acids, about 100 to about 750 amino acids, about 100 to about 500 amino acids, or about 100 to about 300 amino acids. In some embodiments the protein antigen comprises about 200 to 5000 amino acids, about 200 to 4500 amino acids, about 200 to 4000 amino acids, about 200 to 3500 amino acids, about 200 to 3000 amino acids, about 200 to about 2500 amino acids, about 200 to about 2000 amino acids, about 200 to about 1500 amino acids, about 200 to 1000 amino acids, about 300 to about 900 amino acids, about 400 to about 800 amino acids, or about 500 to about 700 amino acids. In some embodiments the protein antigen comprises about 250 to 5000 amino acids, about 500 to 5000 amino acids, about 750 to 5000 amino acids, about 1000 to 5000 amino acids, about 1500 to 5000 amino acids, about 2000 to 5000 amino acids, about 2500 to about 5000 amino acids, about 3000 to about 5000 amino acids, about 3500 to about 5000 amino acids, or about 4000 to 5000 amino acids. In some embodiments, the protein antigen comprises about 100 to about 3000 amino acids, about 250 to about 2750 amino acids, about 400 to about 2500 amino acids, about 500 to about 2500 amino acids, about 750 to about 2500 amino acids, about 1000 to about 2500 amino acids, or about 1500 to about 2500 amino acids.

In some embodiments, the protein antigen has a molecule weight (MW) of about 10 kDa to about 500 kDa. In some embodiments, the protein antigen has a molecule weight (MW) of about 10 kDa to about 500 kDa, about 10 kDa to about 450 kDa, about 10 kDa to about 400 kDa, about 10 kDa to about 350 kDa, about 10 kDa to about 300 kDa, about 10 kDa to about 250 kDa, about 10 kDa to about 200 kDa, about 10 kDa to about 150 kDa, about 10 kDa to about 100 kDa, about 10 kDa to about 50 kDa. In some embodiments, the protein antigen has a MW of about about 20 kDa to about 500 kDa, about 20 kDa to about 450 kDa, 20 kDa to about 400 kDa, 20 kDa to about 350 kDa, about 20 kDa to about 300 kDa, about 20 kDa to about 250 kDa, about 20 kDa to about 200 kDa, about 20 kDa to about 150 kDa, about 20 kDa to about 100 kDa, or about 20 kDa to about 50 kDa. In some embodiments, the protein antigen has a MW of about 30 kDa to about 500 kDa, about 30 kDa to about 450 kDa, about 30 kDa to about 400 kDa, 30 kDa to about 350 kDa, about 30 kDa to about 300 kDa, about 30 kDa to about 250 kDa, or about 30 kDa to about 200 kDa. In some embodiments, the protein antigen has a MW of about about 50 kDa to about 500 kDa, about 50 kDa to about 450 kDa, 50 kDa to about 400 kDa, about 50 kDa to about 350 kDa, about 50 kDa to about 300 kDa, about 50 kDa to about 250 kDa, or about 50 kDa to about 200 kDa, about 50 kDa to about 150 kDa, or about 50 kDa to about 100 kDa. In some embodiments, the protein antigen has a MW of about about 75 kDa to about 500 kDa, about 75 kDa to about 450 kDa, 75 kDa to about 400 kDa, about 75 kDa to about 350 kDa, about 75 kDa to about 300 kDa, about 75 kDa to about 250 kDa, about 75 kDa to about 200 kDa, about 75 kDa to about 150 kDa, or about 75 kDa to about 100 kDa. In some embodiments, the protein antigen has a MW of about about 100 kDa to about 500 kDa, about 100 kDa to about 450 kDa, 100 kDa to about 400 kDa, about 100 kDa to about 350 kDa, about 100 kDa to about 300 kDa, about 100 kDa to about 250 kDa, about 100 kDa to about 200 kDa, or about 100 kDa to about 150 kDa. In some embodiments, the protein antigen has a MW of about about 150 kDa to about 500 kDa, about 150 kDa to about 450 kDa, 150 kDa to about 400 kDa, about 150 kDa to about 350 kDa, about 150 kDa to about 300 kDa, about 150 kDa to about 250 kDa, about 150 kDa to about 200 kDa. In some embodiments, the protein antigen has a MW of about 200 kDa to about 500 kDa, about 200 kDa to about 450 kDa, about 200 kDa to about 400 kDa, about 200 kDa to about 350 kDa, about 200 kDa to about 300 kDa, or about 200 kDa to about 250 kDa. In some embodiments, the protein antigen has a MW of about 250 kDa to about 500 kDa, about 250 kDa to about 450 kDa, about 250 kDa to about 400 kDa, about 250 kDa to about 350 kDa, or about 250 kDa to about 300 kDa. In some embodiments, the protein antigen has a MW of about 300 kDa to about 500 kDa, about 300 kDa to about 450 kDa, about 300 kDa to about 400 kDa, or about 300 kDa to about 350 kDa. In some embodiments, the protein antigen has a MW of about 350 kDa to about 500 kDa, about 350 kDa to about 450 kDa, or about 350 kDa to about 400 kDa. In some embodiments, the protein antigen has a MW of about 400 kDa to about 500 kDa, or about 400 kDa to about 450 kDa.

In some embodiments, the protein antigen is a monomeric antigen (i.e., a single antigenic polypeptide chain).

In some embodiments, the protein antigen is a multimeric antigen, e.g., a dimer, trimer, tetramer, pentamer, hexamer, septamer, octamer, or decamer. In some embodiments, the protein antigen is a dimer antigen. In some embodiments, the protein antigen is a trimer antigen. In some embodiments, the multimeric antigen comprises identical monomer subunits, i.e., repeating sequences of the same antigen, such as two repeating sequences of the same antigen (i.e., a homodimer antigen) or three repeating sequences of the same antigen (i.e., a homotrimer antigen). In some embodiments, the multimeric antigen comprises different monomer subunits, i.e., different protein antigen sequences from the same pathogen, such as two different sequences from the same pathogen (i.e., a heterodimer antigen) or three different sequences from the same pathogen (i.e., a heterotrimer antigen). In some embodiments, two or more of the protein antigen sequences of the monomer subunits of the multimeric antigen are each from different pathogens.

In some embodiments, the protein antigen can be derived from a virus, bacterium, parasite, plant, protozoan, fungus.

Suitable antigenic peptides or protein antigens are commonly known in the art and are available from commercial, government, and scientific sources. The antigens may be purified or partially purified polypeptides derived from viral or bacterial sources. The antigens can be recombinant polypeptides produced by expressing DNA encoding the polypeptide antigen in a heterologous expression system.

In some embodiments, antigenic peptide or protein antigen can be from a virus, including but not limited to a virus from any of the following viral families: Arenaviridae, Arterivirus, Astroviridae, Baculoviridae, Badnavirus, Barnaviridae, Birnaviridae, Bromoviridae, Bunyaviridae, Caliciviridae, Capillovirus, Carlavirus, Caulimovirus, Circoviridae, Closterovirus, Comoviridae, Coronaviridae (e.g., Coronavirus, such as severe acute respiratory syndrome (SARS) virus), Corticoviridae, Cystoviridae, Deltavirus, Dianthovirus, Enamovirus, Filoviridae (e.g., Marburg virus and Ebola virus (e.g., Zaire, Reston, Ivory Coast, or Sudan strain)), Flaviviridae, (e.g., Hepatitis C virus, Dengue virus 1, Dengue virus 2, Dengue virus 3, and Dengue virus 4), Hepadnaviridae, Herpesviridae (e.g., Human herpesvirus 1, 3, 4, 5, and 6, and Cytomegalovirus), Hypoviridae, Iridoviridae, Leviviridae, Lipothrixviridae, Microviridae, Orthomyxoviridae (e.g., Influenzavirus A and B and C), Papovaviridae, Paramyxoviridae (e.g., measles, mumps, and human respiratory syncytial virus), Parvoviridae, Picornaviridae (e.g., poliovirus, rhinovirus, hepatovirus, and aphthovirus), Poxviridae (e.g., vaccinia and smallpox virus), Reoviridae (e.g., rotavirus), Retroviridae (e.g., lentivirus, such as human immunodeficiency virus (HIV) 1 and HIV 2), Rhabdoviridae (for example, rabies virus, measles virus, respiratory syncytial virus, etc.), Togaviridae (for example, rubella virus, dengue virus, etc.), and Totiviridae. Suitable viral antigens also include all or part of Dengue protein M, Dengue protein E, Dengue D1NS1, Dengue D1NS2, and Dengue D1NS3.

Viral antigens may be derived from a particular strain such as a papilloma virus, a herpes virus, e.g., herpes simplex 1 and 2; a hepatitis virus, for example, hepatitis A virus (HAV), hepatitis B virus (HBV), hepatitis C virus (HCV), the delta hepatitis D virus (HDV), hepatitis E virus (HEV) and hepatitis G virus (HGV), the tick-borne encephalitis viruses; parainfluenza, varicella-zoster, cytomeglavirus, Epstein-Barr, rotavirus, rhinovirus, adenovirus, coxsackieviruses, equine encephalitis, Japanese encephalitis, yellow fever, Rift Valley fever,and lymphocytic choriomeningitis.

In some embodiments, the antigenic peptide or protein antigen can be from a bacteria, including but not limited to a bacteria from any of the following families: Actinomyces, Anabaena, Bacillus, Bacteroides, Bdellovibrio, Bordetella, Borrelia, Campylobacter, Caulobacter, Chlamydia, Chlorobium, Chromatium, Clostridium, Corynebacterium, Cytophaga, Deinococcus, Escherichia, Francisella, Halobacterium, Heliobacter, Haemophilus, Hemophilus influenza type B (HIB), Hyphomicrobium, Legionella, Leptspirosis, Listeria, Meningococcus A, B and C, Methanobacterium, Micrococcus, Myobacterium, Mycoplasma, Myxococcus, Neisseria, Nitrobacter, Oscillatoria, Prochloron, Proteus, Pseudomonas, Phodospirillum, Rickettsia, Salmonella, Shigella, Spirillum, Spirochaeta, Staphylococcus, Streptococcus, Streptomyces, Sulfolobus, Thermoplasma, Thiobacillus, and Treponema, Vibrio, and Yersinia.

In some embodiments, the antigenic peptide or protein antigen can be from a parasite, including but not limited to a parasite from any of the following families: Cryptococcus neoformans, Histoplasma capsulatum, Candida albicans, Candida tropicalis, Nocardia asteroides, Rickettsia ricketsii, Rickettsia typhi, Mycoplasma pneumoniae, Chlamydial psittaci, Chlamydial trachomatis, Plasmodium falciparum, Trypanosoma brucei, Entamoeba histolytica, Toxoplasma gondii, Trichomonas vaginalis and Schistosoma mansoni. These include Sporozoan antigens, Plasmodian antigens, such as all or part of a Circumsporozoite protein, a Sporozoite surface protein, a liver stage antigen, an apical membrane associated protein, or a Merozoite surface protein.

In some embodiments, the protein antigen or antigenic peptide comprises a human immunodeficiency virus (HIV) antigen, a SARS-CoV-2 antigen, an influenza antigen, a rotavirus antigen, a cytomegalovirus (CMV) antigen, an Epstein-Barr virus antigen, a respiratory syncytial virus (RSV) antigen, or a cholera antigen. In some embodiments, the protein antigen comprises a human immunodeficiency virus (HIV) antigen, a SARS-CoV-2 antigen, an influenza antigen, a rotavirus antigen, a cytomegalovirus (CMV) antigen, an Epstein-Barr virus antigen, a respiratory syncytial virus (RSV) antigen, or a cholera antigen. In some embodiments, the antigenic peptide comprises a human immunodeficiency virus (HIV) antigen, a SARS-CoV-2 antigen, an influenza antigen, a rotavirus antigen, a cytomegalovirus (CMV) antigen, an Epstein-Barr virus antigen, a respiratory syncytial virus (RSV) antigen, or a cholera antigen.

In some embodiments, the protein antigen comprises an HIV antigen. In some embodiments, the protein antigen comprises a SARS-CoV-2 antigen. In some embodiments, the protein antigen comprises an influenza antigen. In some embodiments, the protein antigen comprises a rotavirus antigen. In some embodiments, the protein antigen comprises a CMV antigen. In some embodiments, the protein antigen comprises a cholera antigen. In some embodiments, the antigenic peptide comprises an HIV antigen. In some embodiments, the antigenic peptide comprises a SARS-CoV-2 antigen. In some embodiments, the antigenic peptide comprises an influenza antigen. In some embodiments, the antigenic peptide comprises a rotavirus antigen. In some embodiments, the antigenic peptide comprises a CMV antigen. In some embodiments, the antigenic peptide comprises a cholera antigen.

In some embodiments, the amino acid sequence of the antigenic peptide or protein antigen may be naturally existing amino acid sequence of the antigen. In some embodiments, the antigenic peptide or protein antigen may be a sequence modified from the naturally existing amino acid sequence of the antigen. The modifications may serve to enhance antigenicity or improve production of the amphiphilic conjugate.

Human immunodeficiency virus (HIV) antigens are commonly known in the art. Non-limiting examples of HIV antigens may be found in Jardine et al (2015) (Priming a broadly neutralizing antibody response to HIV-1 using a germline-targeting immunogen. Science. 349(6244):156-61); Kim et al (2021) (Current approaches to HIV vaccine development: a narrative review. J Int AIDS Soc., 24: e25793); and Haynes et al (2023) (Strategies for HIV-1 vaccines that induce broadly neutralizing antibodies. Nat Rev Immunol 23, 142-158), the entire contents of each of which are incorporated herein by reference.

In some embodiments, the HIV antigen comprises or consists of an HIV envelope protein (Env) antigen. In some embodiments, the HIV Env antigen is a gp120 antigen or gp140 antigen. In some embodiments, the HIV Env antigen is a gp120 antigen. In some embodiments, the HIV Env antigen is gp120 engineered outer domain-germ line-targeting immunogen 8 (eOD-GT8). In some embodiments, the eOD-GT8 gp120 antigen comprises or consist of the amino acid sequence of SEQ ID NO: 1.

In some embodiments, the HIV envelope protein antigen is a native-like trimer antigen (of repeating monomers) that mimics the structure of the virion-associated spike (e.g., HIV MD39 SOSIP). In some embodiments, the monomer that makes up the HIV evelope protein antigen trimer MD39 SOSIP comprises or consists of the amino acid sequence of SEQ ID NO: 3.

SARS-CoV-2 antigens are commonly known in the art. Examples of SARS-CoV-2 antigens used for existing vaccine technology as well as those under testing and experimentation may be found in Poland et al (2020) (SARS-Cov-2 Immunity: Review and Applications to Phase 3 Vaccine Candidates. Lancet 396:1595-606); Dalvie et al (2021) (Engineered SARS-CoV-2 receptor binding domain improves manufacturability in yeast and immunogenicity in mice. Proc. Natl. Acad. Sci. U.S.A. 118, e2106845118), and Jang et al (2020) (A vaccine targeting the RBD of the S protein of SARS-CoV-2 induces protective immunity. Nature 586, 572-577), which are incorporated herein by reference.

In some embodiments, the SARS-CoV-2 antigen comprises a SARS-CoV-2 spike protein (also known as “S protein”), or an antigenic fragment of the spike protein. In some embodiments, the SARS-CoV-2 antigen comprises an antigen from the S1 subunit of the spike protein. In some embodiments, the SARS-CoV-2 antigen comprises an antigen from the N-terminal domain of the spike protein. In some embodiments, the SARS-CoV-2 antigen comprises an antigen from the receptor binding domain (RBD) of the spike protein. In some embodiments, the SARS-CoV-2 antigen comprises an antigen of the S2 subunit of the spike protein.

In some embodiments, the protein antigen comprises the receptor binding domain (RBD) of the SARS-CoV-2 spike protein, or an antigen derived from the RBD. In some embodiments, the SARS-CoV-2 RBD protein antigen comprises or consists of the amino acid sequence of SEQ ID NO: 2.

Influenza antigens are commonly known in the art and may be found in Gomez Lorenzo et al (2013) (Immunobiology of influenza vaccines. Chest. 143(2):502-510; Rao et al (2010) Comparative efficacy of hemagglutinin, nucleoprotein, and matrix 2 protein gene-based vaccination against H5N1 influenza in mouse and ferret. PLoS One. 5(3):e9812), incorporated herein by reference. In some embodiments, the influenza antigen comprises a hemagglutinin (HA) antigen, a neuraminidase antigen, a nucleoprotein (NP) antigen or an ion channel matrix protein (M2) antigen.

Rotavirus antigens are commonly known in the art. Teachings of antigens known to elicit expression of antibodies, particularly neutralizing antibodies, against rotarovirus may be found in, e.g., U.S. Pat. No. 7,311,918B2, US 6,16431, and Dennehy (2008) (Rotavirus vaccines: an overview. Clin Microbiol Rev. 21(1):198-208), incorporated herein by reference. In some embodiments, the rotarovirus antigen comprises a VP4 antigen, VP6 antigen, or VP7 antigen.

Cytomegalovirus (CMV) antigens are commonly known in the art. Teachings of CMV antigens may be found at, e.g., Nelson et al (2018) (A new era in cytomegalovirus vaccinology: considerations for rational design of next-generation vaccines to prevent congenital cytomegalovirus infection. npj Vaccines 3, 38), incorporated herein by reference. Neutralizing antibodies targeting proteins gB, gH, and UL128-131A of CMV have been found after natural infection. In some embodiments, the CMV antigen comprises a gB antigen, gH antigen, or a UL128-131A antigen.

Currently, no vaccines against Epstein-Barr virus (EBV) have been successfully developed, though EBV antigens that elicit antibody production, particularly neutralizing antibody production, are commonly known in the art. Teachings of known EBV antigens may be found at Cui et al (2021) (Epstein Barr Virus: Development of Vaccines and Immune Cell Therapy for EBV-Associated Diseases. Front. Immunol., Vol 12), incorporated herein by reference. In some embodiments, the EBV antigen comprises a gp350 antigen, a gH antigen, a gL antigen, or gB antigen.

Most recent attempts to generate an respiratory syncytial virus (RSV) vaccine have been based on the F protein of RSV, as the F protein mediates virus entry into host cells and an anti-F antibody has been shown to reduce severe RSV disease in high-risk infants. Other proteins which have been shown to be capable of eliciting neutralizing antibodies include the N and M2-1 protein. Teachings of known RSV antigens may be found at, e.g., Ciconi et al (2020) (First-in-Human Randomized Study to Assess the Safety and Immunogenicity of an Investigational Respiratory Syncytial Virus (RSV) Vaccine Based on Chimpanzee-Adenovirus-155 Viral Vector-Expressing RSV Fusion, Nucleocapsid, and Antitermination Viral Proteins in Healthy Adults, Clinical Infectious Diseases, 70(10): 2073-2081) and Graham et al (2015) (Novel antigens for RSV vaccines. Curr Opin Immuno1.35:30-8), incorporated herein by reference. In some embodiments, the RSV antigen comprises a F protein antigen, an N protein antigen, or an M2-1 protein antigen.

Research on immune response to cholera (e.g., Vibrio cholerae) infection has focuses primarily on antibodies. Antibody responses have been found against the 0-specific polysaccharide of V. cholerae, as well as against the A subunit (CtxA) and B subunit (CtxB) of cholera toxin (see Harris (2018) Cholera: Immunity and Prospects in Vaccine Development. J Infect Dis. 15; 218(suppl_3):S141-S146; incorporated herein by reference). In some embodiments, the cholera antigen comprises the O-specific polysaccharide of V. cholerae, a CtxA antigen, or a CtxB antigen.

In some embodiments, the immunogen is a polysaccharide antigen. Polysaccharides are major components on the surface of bacteria. Polysaccharide-encapsulated bacteria are the leading cause for several serious bacterial infection in childen, such as bacterial meningitis and pneumonia. The polysaccharide capsules of bacteria determine their virulence, and therefore targeting their capsidal polysaccharide can confer significant protection against bacteria infections.

Bacterial polysaccharides are very heterogeneous within and between species, and they are also T-lymphocyte independent antigens. With a few exceptions, immunization with free polysaccharides generally stimulates short-lived B-cell responses and can even result in hyporesponsiveness to future vaccine doses. Recent studies suggest that polysaccharide conjugates may induce T-cell dependent response and stronger B-cell response, resulting in long-term immunity (see, e.g., Pollard et al (2009) Maintaining protection against invasive bacteria with protein-polysaccharide conjugate vaccines. Nat Rev Immunol 9, 213-220).

Polysaccharide antigens are commonly known in the art. Teachings of known polysaccharide antigens, particularly those that have been developed to be polysaccharide vaccines, may be found at, e.g., Perera et al (2021) (Polysaccharide Vaccines: A Perspective on Non-Typhoidal Salmonella” Polysaccharides 2, no. 3: 691-714); and Aithal et al (2012) (PolysacDB: A Database of Microbial Polysaccharide Antigens and Their Antibodies. PLoS ONE 7(4): e34613), the entire contents of each of which are incorporated herein by reference.

In some embodiments, the polysaccharide antigen is a cholera (e.g., Vibrio cholerae) antigen. In some embodiments, the polyssachride antigen is an O-specific polysaccharide of V. cholera.

### (iii) Linkers

In various aspects of the present disclosure, the lipid, e.g., albumin-binding lipid, and the cargo, e.g., immunogen, are connected by a linker molecule. In some embodiments, the linker is covalently conjugated to the lipid, to the cargo, or to both the lipid and the cargo. In embodiments, the linker is disposed between and covalently conjugated to each of the lipid and the cargo.

Depending on the amino acid sequence, some amino-acid based immunogens can be essentially insoluble. Therefore, in certain aspects, a polar block linker is included as a linker between the cargo and the lipid to increase solubility of the amphiphilic conjugate.

In some embodiments, the polar block linker enables the amphiphilic conjugate to bind to albumin. In some embodiments, the polar block linker increase the ability of the amphiphilic conjugate to bind to albumin. In some embodiments, the polar block linker increases the solubility of the conjugate without preventing its ability to bind to albumin.

In some embodiments, the polar block linker modulates (e.g., diminishes, or enhances) the ability of the lipid to insert into the plasma membrane of cells, such as cells adjacent to the mucosal of administration.

One of ordinary skill in the art will recognize that the length and composition of the linker can be adjusted based on the lipid and cargo selected. Additional non-limiting examples of linkers applicable for the amphiphilic conjugate of the present disclosure may be found in WO 2019/060425, the entire contents of which are incorporated herein by reference.

In some embodiments, suitable polar blocks include, but are not limited to, oligonucleotides such as those discussed below, a hydrophilic polymer including but not limited to poly(ethylene glycol) (MW: 500 Da to 20,000 Da), polyacrylamide (MW: 500 Da to 20,000 Da), polyacrylic acid; a string of hydrophilic amino acids such as serine, threonine, cysteine, tyrosine, asparagine, glutamine, aspartic acid, glutamic acid, lysine, arginine, histidine, or combinations thereof; polysaccharides, including but not limited to, dextran (MW: 1,000 Da to 2,000,000 Da); or combinations thereof.

In some embodiments, the polar block, whether a separate component or the cargo itself, provides solubility to the overall lipid conjugate based on the molecular weight of the polar block. For example, in some embodiments, a polar block having a molecular weight of 2,000 Da is sufficient to make the lipid conjugate soluble for albumin binding. In some embodiments, the polar block has a molecular weight of about 300 to about 20,000 Da. In some embodiments, the polar block has a molecular weight of about 1,000 to about 15,000 Da. In some embodiments, the polar block has a molecular weight of about 1,500 to about 10,000 Da. In some embodiments, the polar block has a molecular weight of about 2,000 to about 5,000 Da. In some embodiments, the polar block has a molecular weight of about 1,000 to about 2,500 Da. In some embodiments, the polar block has a molecular weight of about 1,000 to about 3,000 Da. In some embodiments, the polar block has a molecular weight of about 1,000 to about 3,500 Da. In some embodiments, the polar block has a molecular weight of about 1,000 to about 4,000 Da. In some embodiments, the polar block has a molecular weight of about 1,000 to about 5,000 Da. In some embodiments, the polar block has a molecular weight of about 5,000 to about 10,000 Da. In some embodiments, the polar block has a molecular weight of about 15,000 to about 20,000 Da.

In some embodiments, the hydrophobic lipid and the linker/cargo are covalently linked. In some embodiments, the covalent bond is a non-cleavable linkage or a cleavable linkage. In some embodiments, the non-cleavable linkage includes an amide bond or phosphate bond, and the cleavable linkage includes a disulfide bond, acid-cleavable linkage, ester bond, anhydride bond, biodegradable bond, or enzyme-cleavable linkage.

Ethylene Glycol (EG): In certain embodiments, the linker (first and/or second linker) comprises one or more ethylene glycol (EG) units, more preferably two or more EG units (i.e., polyethylene glycol (PEG)). For example, in certain embodiments, an amphiphilic conjugate includes a cargo (i.e., peptide antigen or protein antigen) and a hydrophobic lipid (e.g., albumin-binding lipid) linked by a polyethylene glycol (PEG) molecule or a derivative or analog thereof.

In some embodiments, amphiphilic conjugates suitable for use in the methods disclosed herein contain an immunogen, e.g., antigenic peptide or protein antigen, covalently linked to PEG which is in turn covalently linked to a hydrophobic lipid, e.g., albumin-binding lipid. The precise number of EG units depends on the lipid and the cargo.

In some embodiments, the linker (e.g., first linker) comprises a PEG molecule (e.g., first PEG molecule) or other similarly soluble polymer. The PEG molecule (e.g., first PEG molecule) is a repeating unit of polyethylene glycol represented as (PEG),, where n represents the number of repeating PEG monomers (i.e., EG units). In some embodiments, the number of repeating PEG monomers (n) in the PEG molecule can be between about 1 and about 150, between about 1 and about 125, between about 1 and about 100, between about 1 and about 50, between about 50 and about 100, between about 100 and about 150. In some embodiments the number of repeating PEG monomers (ne) in the PEG molecule can be between about 10 and about 90, between about 20 and about 80, between about 30 and about 70, or between about 40 and about 60 monomers. In certain embodiments, the number of repeating PEG monomers (n) in the PEG molecule can be between about 45 and about 150. In certain embodiments, the number of repeating PEG monomers in the PEG linker (e.g., first linker) is between about 45 and 55 monomers. For example, in certain embodiments, the number of repeating PEG monomers in the PEG linker (e.g., first linker) is about 48 monomers.

In some embodiments, the PEG linker (e.g., first linker) or PEG molecule has a molecular weight of about 300-20,000 daltons. In some embodiments, the PEG linker or PEG molecule has a molecular weight of about 1,000 daltons. In some embodiments, the PEG linker or PEG molecule has a molecular weight of about 1,500 daltons. In some embodiments, the PEG linker or PEG molecule has a molecular weight of about 2,000 daltons. In some embodiments, the PEG linker or PEG molecule has a molecular weight of about 2,500 daltons. or PEG molecule In some embodiments, the PEG linker or PEG molecule has a molecular weight of about 3,500 daltons. In some embodiments, the PEG linker or PEG molecule has a molecular weight of about 4,000 daltons. In some embodiments, the PEG linker or PEG molecule or PEG molecule has a molecular weight of about 5,000 daltons. In some embodiments, the PEG linker or PEG molecule has a molecular weight of about 6,000 daltons. In some embodiments, the PEG linker or PEG molecule has a molecular weight of about 7,000 daltons. In some embodiments, the PEG linker or PEG molecule has a molecular weight of about 8,000 daltons. In some embodiments, the PEG linker or PEG molecule has a molecular weight of about 9,000 daltons. In some embodiments, the PEG linker or PEG molecule has a molecular weight of about 10,000 daltons. In some embodiments, the PEG linker or PEG molecule has a molecular weight of about 11,000 daltons. In some embodiments, the PEG linker or PEG molecule has a molecular weight of about 12,000 daltons. In some embodiments, the PEG linker or PEG molecule has a molecular weight of about 13,000 daltons. In some embodiments, the PEG linker or PEG molecule has a molecular weight of about 14,000 daltons. In some embodiments, the PEG linker or PEG molecule has a molecular weight of about 15,000 daltons. In some embodiments, the PEG linker or PEG molecule has a molecular weight of about 16,000 daltons. In some embodiments, the PEG linker or PEG molecule has a molecular weight of about 17,000 daltons. In some embodiments, the PEG linker or PEG molecule has a molecular weight of about 18,000 daltons. In some embodiments, the PEG linker or PEG molecule has a molecular weight of about 19,000 daltons. In some embodiments, the PEG linker or PEG molecule has a molecular weight of about 20,000 daltons.

Second linker: In some embodiments, the cargo of the amphiphilic conjugate is a large protein antigen (e.g., a dimer or trimer antigen) and requires a longer linker to avoid steric hindrance of the large protein antigen in the amphiphilic conjugate. In such cases, in some embodiments a second linker is conjugated to a first linker to form a suitable linker for the large protein antigen, wherein the first linker is any linker described above. The second linker and the first linker are conjugated to each other, directly or indirectly (e.g., conjugated via click chemistry), and are disposed between the lipid and the cargo.

In some embodiments, the second linker is diposed between and connects the first linker and the cargo. In some embodiments, the second linker is disposed between and connects the first linker and the lipid.

In some embodiments, the second linker comprises a PEG molecule, e.g., a second PEG molecule (e.g., a second repeating unit of PEG monomers). In some embodiments the PEG molecule of the second linker is the same as the PEG molecule in the first linker. In some embodiments the PEG molecule of the second linker is different from the PEG molecule in the first linker. In some embodiments, the number of repeating PEG monomers (m) in the second linker can be 1 to 20 monomers. In some embodiments, the number of repeating PEG monomers (m) in the second linker can be 2 to 18, 5 to 15, or 8 to 12 monomers. In some embodiments, the number of repeating PEG monomers (m) in the second linker is 4 monomers.

In some embodiments the second linker comprises a dibenzocyclooctyne (DBCO) group (or an equivalent functional group) linked to a PEG molecule, e.g., a second PEG molecule (e.g., a second repeating unit of PEG monomers). In some embodiments the second linker can be represented as dibenzocyclooctyne-(PEG)ₘ (or DBCO-(PEG)ₘ), wherein m represents the number of repeating PEG monomers. In some embodiments, the number of repeating PEG monomers (m) in the second linker can be 1 to 20 monomers. In some embodiments, the number of repeating PEG monomers (m) in the second linker can be 2 to 18, 5 to 15, or 8 to 12 monomers. In some embodiments, the number of repeating PEG monomers (m) in the second linker is 4 monomers.

In some embodiments, the second linker further comprises a maleimide group. In some embodiments, the second linker comprises DBCO-(PEG)ₘ-maleimide. In certain embodiments, the second linker comprises DBCO-(PEG)₄-maleimide. The structure of DBCO-(PEG)₄-maleimide is shown below.

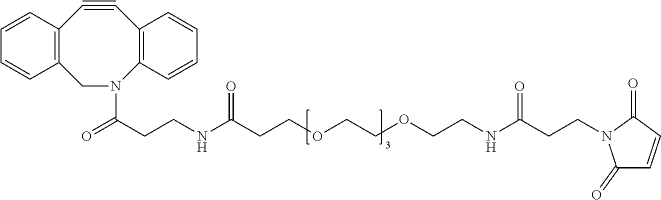

A representative schematic of the structure of a non-limiting example of an amphiphilic conjugate comprising a DBCO-(PEG)₄-maleimide second linker is shown below:

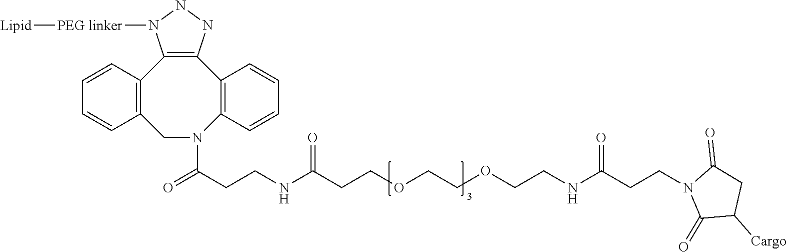

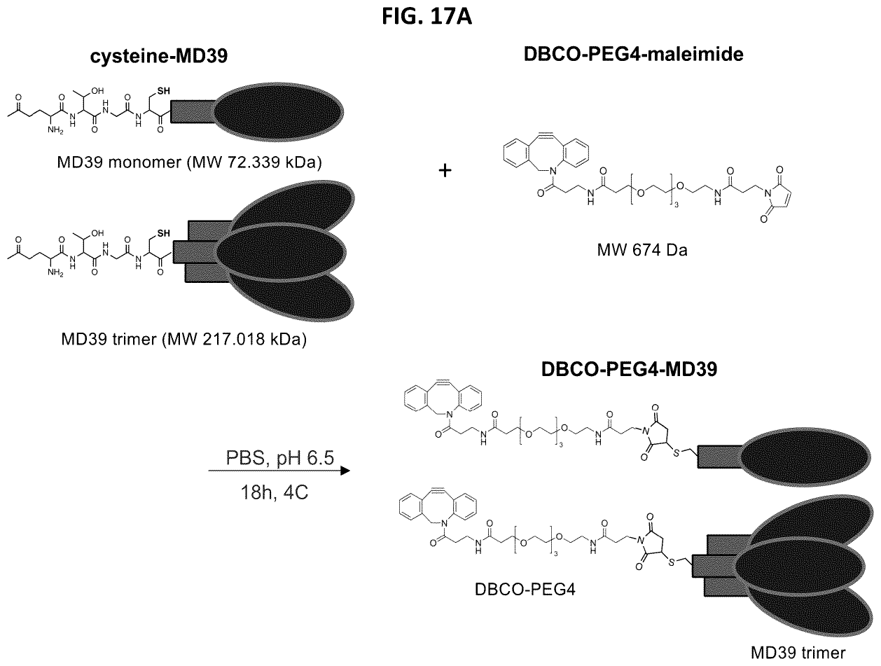

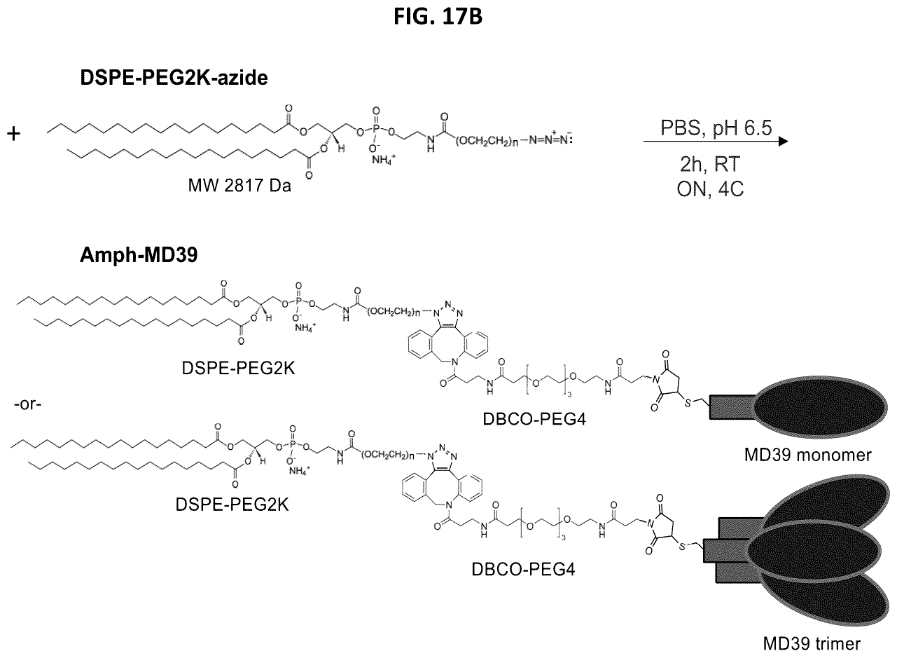

Non-limiting examples of amphiphilic conjugates comprising a DBCO-(PEG)₄-maleimide (DSPE-PEG2K-DBCO-PEG4-MD39) are depicted in FIG. 17B.

Oligonucleotide Linkers. In certain embodiments, the linker is an oligonucleotide. Non-limiting examples of oligonucleotide linkers applicable for the amphiphilic conjugate of the present disclosure may be found in WO 2019/060425, the entire contents of which are incorporated herein by reference. The linker can have any sequence, for example, the sequence of the oligonucleotide can be a random sequence, or a sequence specifically chosen for its molecular or biochemical properties (e.g., highly polar). In certain embodiments, the polar block linker includes one or more series of consecutive adenine (A), cytosine (C), guanine (G), thymine (T), uracil (U), or analog thereof. In certain embodiments, the polar block linker consists of a series of consecutive adenine (A), cytosine (C), guanine (G), thymine (T), uracil (U), or analog thereof.

In certain embodiments, the linker is one or more guanines, for example between 1-10 guanines. It has been discovered that altering the number of guanines between a cargo such as a CpG oligonucleotide, and a lipid tail controls micelle stability in the presence of serum proteins. Therefore, the number of guanines in the linker can be selected based on the desired affinity of the conjugate of the present disclosure for serum proteins such as albumin. It has been previously shown that when the cargo in an amphilic conjugate is a CpG immunostimulatory oligonucleotide and the lipid tail is a diacyl lipid, the number of guanines affects the ability of micelles formed in aqueous solution to dissociate in the presence of serum: 20% of the non-stabilized micelles (lipo-G0T10-CG) were intact, while the remaining 80% were disrupted and bonded with FBS components. In the presence of guanines, the percentage of intact micelles increased from 36% (lipo-G2T8-CG) to 73% (lipo-G4T6-CG), and finally reached 90% (lipo-G6T4-CG). Increasing the number of guanines to eight (lipo-G8T2-CG) and ten (lipo-G10T0-CG) did not further enhance micelle stability. Therefore, in certain embodiments, the linker in a conjugate suitable for use in the methods disclosed herein can include 0, 1, or 2 guanines.

### Methods of Preparing Amphiphilic Conjugates

### (i) Methods of Preparing Antigenic Peptides or Protein Antigens

In some embodiments, the antigenic peptide or protein antigen described herein for use in the amphiphilic conjugates are made in transformed host cells using recombinant nucleic acid, e.g., DNA or RNA, techniques. To do so, a recombinant nucleic acid molecule coding for the antigenic peptide or protein antigen is prepared. Methods of preparing such nucleic acid molecules are well known in the art. For example, sequences coding for the antigenic peptides or protein antigens can be excised from a nucleic acid molecule using suitable restriction enzymes. Alternatively, the nucleic acid molecule can be synthesized using chemical synthesis techniques, such as the phosphoramidate method. A combination of these techniques can be used.

The methods of making an antigenic peptide or protein antigen also include preparing a vector capable of expressing the antigenic peptide or protein antigen in an appropriate host. The vector comprises the nucleic acid molecule that codes for the peptide or protein antigen operatively linked to appropriate expression control sequences. Methods of affecting this operative linking, either before or after the nucleic acid molecule is inserted into the vector, are well known in the art. Expression control sequences include promoters, activators, enhancers, operators, ribosomal nuclease domains, start signals, stop signals, cap signals, polyadenylation signals, and other signals involved with the control of transcription or translation. The resulting vector comprising the nucleic acid molecule encoding the peptide or protein antigen is used to transform an appropriate host. This transformation may be performed using methods well known in the art.

Any of a large number of available and well-known host cells may be suitable for use in the methods disclosed herein. The selection of a particular host is dependent upon a number of factors recognized by the art. These include, for example, compatibility with the chosen expression vector, toxicity of the peptides encoded by the nucleic acid molecule, rate of transformation, ease of recovery of the peptides, expression characteristics, bio-safety and costs. A balance of these factors must be struck with the understanding that not all hosts may be equally effective for the expression of a particular nucleic acid sequence. Within these general guidelines, useful microbial hosts include bacteria (such as E. coli sp.), yeast (such as Saccharomyces sp.) and other fungi, insects, plants, mammalian (including human) cells in culture, or other hosts known in the art.

Next, the transformed host is cultured and purified. Host cells may be cultured under conventional fermentation conditions so that the desired compounds are expressed. Such fermentation conditions are well known in the art. Finally, the antigenic peptides or protein antigens are purified from the cells or culture medium by methods well known in the art.

The antigenic peptides or protein antigens may also be prepared by synthetic methods. For example, solid phase synthesis techniques may be used. Suitable techniques are well known in the art, and include those described in Merrifield (1973), Chem. Polypeptides, pp. 335-61 (Katsoyannis and Panayotis eds.); Merrifield (1963), J. Am. Chem. Soc. 85: 2149; Davis et al. (1985), Biochem. Intl. 10: 394-414; Stewart and Young (1969), Solid Phase Peptide Synthesis; U.S. Pat. No. 3,941,763; Finn et al. (1976), The Proteins (3rd ed.) 2: 105-253; and Erickson et al. (1976), The Proteins (3rd ed.) 2: 257-527, the entire contents of each of which are incorporated by reference herein. Solid phase synthesis is the preferred technique of making individual peptides since it is the most cost-effective method of making small peptides. Compounds that contain derivatized peptides or which contain non-peptide groups may be synthesized by well-known organic chemistry techniques.

Other methods of nucleic acid expression and synthesis are generally known to one of ordinary skill in the relevant art.

The nucleic acid molecules described above can be contained within a vector that is capable of directing their expression in, for example, a cell that has been transduced with the vector. Accordingly, expression vectors containing a nucleic acid molecule encoding a peptide or protein antigen and cells transfected with these vectors are among the embodiments provided herein.

Vectors suitable for use include T7-based vectors for use in bacteria (see, for example, Rosenberg et al., Gene 56: 125, 1987), the pMSXND expression vector for use in mammalian cells (Lee and Nathans, J. Biol. Chem. 263:3521, 1988), and baculovirus-derived vectors (for example the expression vector pBacPAKS from Clontech, Palo Alto, Calif.) for use in insect cells. The nucleic acid inserts, which encode the polypeptide of interest in such vectors, can be operably linked to a promoter, which is selected based on, for example, the cell type in which expression is sought. For example, a T7 promoter can be used in bacteria, a polyhedrin promoter can be used in insect cells, and a cytomegalovirus or metallothionein promoter can be used in mammalian cells. Also, in the case of higher eukaryotes, tissue-specific and cell type-specific promoters are widely available. These promoters are so named for their ability to direct expression of a nucleic acid molecule in a given tissue or cell type within the body. Skilled artisans are well aware of numerous promoters and other regulatory elements which can be used to direct expression of nucleic acids.

In addition to sequences that facilitate transcription of the inserted nucleic acid molecule, vectors can contain origins of replication, and other genes that encode a selectable marker. For example, the neomycin-resistance (neor) gene imparts G418 resistance to cells in which it is expressed, and thus permits phenotypic selection of the transfected cells. Those of skill in the art can readily determine whether a given regulatory element or selectable marker is suitable for use in a particular experimental context.

Viral vectors that are suitable for use include, for example, retroviral, adenoviral, and adeno-associated vectors, herpes virus, simian virus 40 (SV40), and bovine papilloma virus vectors (see, for example, Gluzman (Ed.), Eukaryotic Viral Vectors, CSH Laboratory Press, Cold Spring Harbor, N.Y.).

Prokaryotic or eukaryotic cells that contain and express a nucleic acid molecule that encodes a peptide or protein antigen are also suitable for use. A cell is a transfected cell, i.e., a cell into which a nucleic acid molecule, for example a nucleic acid molecule encoding a peptide or protein antigen has been introduced by means of recombinant DNA techniques. The progeny of such a cell are also considered suitable for use in the methods disclosed herein.

The precise components of the expression system are not critical. For example, a peptide or protein antigen can be produced in a prokaryotic host, such as the bacterium E. coli, or in a eukaryotic host, such as an insect cell (e.g., an Sf21 cell), or mammalian cells (e.g., COS cells, NIH 3T3 cells, or HeLa cells). These cells are available from many sources, including the American Type Culture Collection (Manassas, Va.). In selecting an expression system, it matters only that the components are compatible with one another. Artisans or ordinary skill are able to make such a determination. Furthermore, if guidance is required in selecting an expression system, skilled artisans may consult Ausubel et al. (Current Protocols in Molecular Biology, John Wiley and Sons, New York, N.Y., 1993) and Pouwels et al. (Cloning Vectors: A Laboratory Manual, 1985 Suppl. 1987).

The expressed peptide or protein antigens can be purified from the expression system using routine biochemical procedures, and can be used, e.g., conjugated to a albumin-binding lipid via a linker, as described herein.

### (ii) Methods of Preparing the Amphiphilic Conjugate

In some aspects, the present disclosure provides methods for assembling the amphiphilic conjugate.

In certain embodiments, a cargo immunogen (e.g., antigen peptide or protein antigen) is covalently conjugated to a linker by reacting a free thiol group of a cysteine residue comprised in the antigen or protein antigen with a reactive maleimide group present in the linker. In some embodiments, the cysteine residue having a free thiol group is at or near the N-terminus of the antigenic peptide or protein antigen. In some embodiments, the cysteine residue having a free thiol group is at or near the N-terminus of the antigenic peptide or protein antigen.

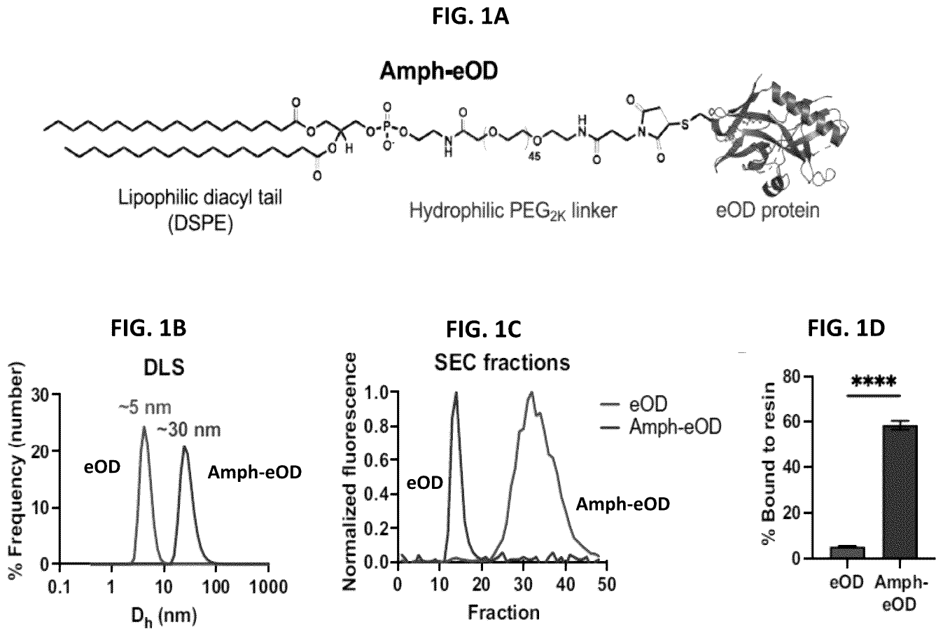

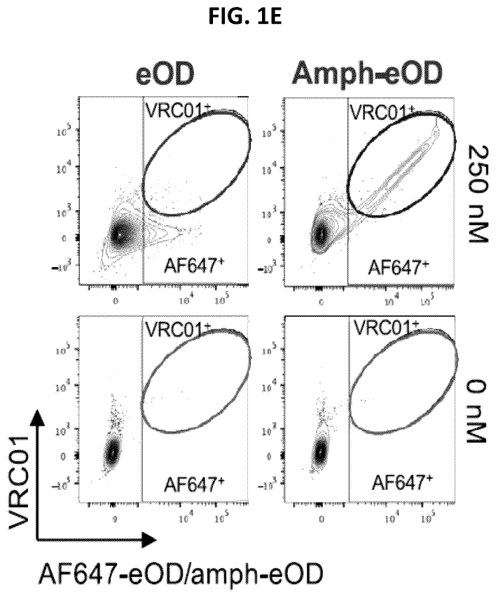

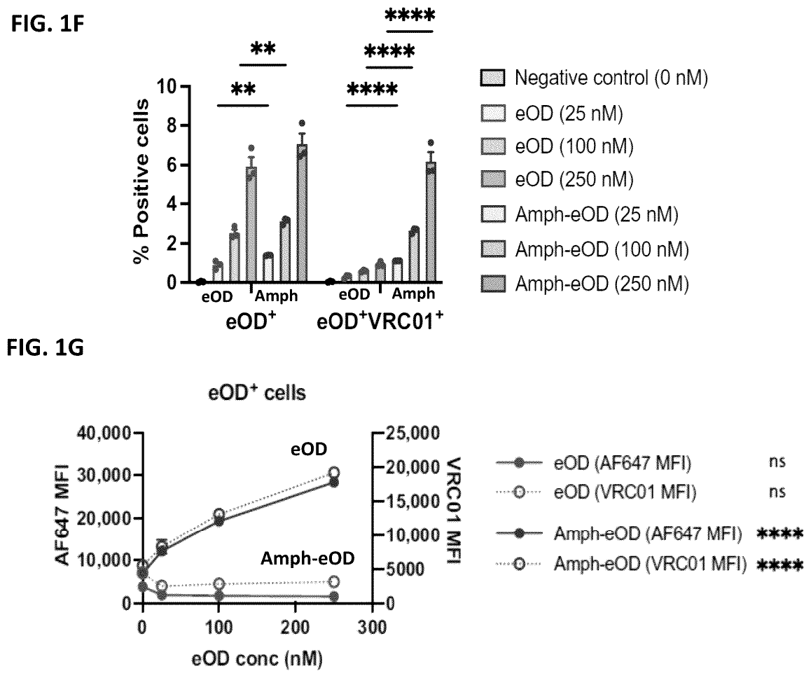

In some embodiments, a cargo immunogen (e.g., antigen peptide or protein antigen) comprising a cysteine residue containing a free thiol group at or near the N-terminus is allowed to react with the malemide group comprised in a lipid-PEG linker-maleimide molecule (e.g., DSPE-PEG2K-maleimide) to form a covalent bond, thereby forming an amphiphilic conjugate (e.g., DSPE-PEG2K-protein antigen, see FIG. 1A).

In some embodiments, a cargo immunogen (e.g., antigen peptide or protein antigen) comprising a cysteine residue containing a free thiol group at or near the N-terminus is allowed to react with the malemide group of a second linker (e.g., DBCO-(PEG)ₘ-maleimide such as DBCO-(PEG)₄-maleimide), forming an intermediate product (e.g., DBCO-(PEG)ₘ-protein antigen). Subsequently, the DBCO group of the intermediate products is allowed to react with a reactive azide group of a lipid-PEG linker-azide molecule (e.g., DSPE-PEG-2K-azide) to form a covalent bond, thereby forming an amphiphilic conjugate (e.g., DSPE-PEG2K-DBCO-PEG4-protein antigen, see FIGS. 17A-17B).

The amphiphilic conjugates of the invention can be purified and characterized using standard methods in the art.

### Methods of Using the Vaccines

In certain aspects, the present disclosure provides methods of vaccinating a subject, comprising transmucosally (e.g., intranasally) administering to the subject a vaccine comprising an amphiphilic conjugate disclosed herein. The present disclosure also provides methods of immunizing a subject, comprising transmucosally (e.g., intranasally) administering to the subject a vaccine comprising an amphiphilic conjugate disclosed herein. Transmucosally (e.g., intranasally) administering the vaccine to the subject induces or enhances an immune response, e.g., humoral immune response or cell-mediated immune response, in the subject. In some embodiments, transmucosally (e.g., intranasally) administering the vaccine induces a greater immune response, e.g., humoral immune response or a cell-mediated immune response, than the peptide or protein antigen alone.

In some embodiments, the method comprises inducing a humoral immune response. In some embodiments, the humoral immune response (e.g., antibody expression) is systemic. In some embodiments, the humoral immune response (e.g., antibody expression) is localized. In some embodiments, the humoral immune response (e.g., antibody expression) is at mucosal surfaces.

In some embodiments, the method comprises inducing production of an antibody that binds to the peptide or protein antigen of the amphiphlilic conjugate. The antibody produced can be an IgG antibody or IgA antibody. In some embodiments, the antibody is an IgG antibody. In some embodiments, the antibody is an IgA antibody. In some embodiments, the antibody is a neutralizing antibody. In some embodiments, the method comprises inducing production of a neutralizing antibody against the pathogenic antigen (e.g., HIV, SARS-CoV2). In some embodiments, the method comprises inducing sustained levels of a neutralizing antibody against the pathogenic antigen (e.g., HIV, SARS-CoV2). In some embodiments, the method comprises inducing increased levels of IgG and/or IgA antibodies in any one or more of serum, upper and/or lower respiratory mucosa, or genitourinary mucosa. In some embodiments, the method comprises inducing increased GC and/or follicular helper T cell (Tfh) responses in the NALT.

In some embodiments, the method comprises inducing a sustained level of antibody (e.g., IgA and/or IgA) titre in the serum, vaginal and/or feces of a subject for at least 10 weeks, 15 weeks, 20 weeks, 25 weeks 30 weeks, 35 weeks, 40 weeks, 45 weeks or 50 weeks. In some embodiments, the method comprises inducing a high level of antibody (e.g., IgA and/or IgG) titre in the serum, vaginal and/or feces of a subject for at least 10 weeks, 15 weeks, 20 weeks, 25 weeks 30 weeks, 35 weeks, 40 weeks, 45 weeks or 50 weeks. In some embodiments, the antibody secreting cells (ASC) that produce the antibody are present in a subject at least 0.5 years, at least 1 year, at least 1.5 years, at least 2 years, at least 3 years, at least 4 years, or at least 5 years after administration of the vaccine. In some embodiments, the ASC cells are detected in the female reproductive tract (FRT) and/or bone marrow (BM).

### (i) Formulations

The present disclosure provides vaccines comprising amphiphilic conjugates disclosed herein. The vaccines are for administration by transmucosal (e.g., nasal, vaginal, rectal, or sublingual) routes. The vaccines can be administered using bioerodible inserts and can be formulated in dosage forms appropriate for each route of administration.

As further studies are conducted, information will emerge regarding appropriate dosage levels for treatment of various conditions in various subjects or patients, and the ordinary skilled worker, considering the therapeutic context, age, and general health of the recipient, will be able to ascertain proper dosing. The selected dosage depends upon the desired therapeutic effect, on the route of administration, and on the duration of the treatment desired.

Formulations for administration to the mucosa can be spray dried drug particles, which may be incorporated into a tablet, gel, capsule, suspension or emulsion. Standard pharmaceutical excipients are available from any formulator.

In some embodiments, the vaccine comprising an amphiphilic conjugate further comprises an adjuvant.

### (ii) Adjuvants

A vaccine comprising an amphiphilic conjugate can be administered alone, or in combination with an adjuvant. In some embodiments, the vaccine can be administered separately from the adjuvant. In some embodiments, the vaccine is formulated together with the adjuvant.

The adjuvant may be, without limitation, alum (e.g., aluminum hydroxide, aluminum phosphate); saponins purified from the bark of the Q. saponaria tree such as QS21 (a glycolipid that elutes in the 21st peak with HPLC fractionation; Antigenics, Inc., Worcester, Mass.); poly[di(carboxylatophenoxy)phosphazene (PCPP polymer; Virus Research Institute, USA); Flt3 ligand; Leishmania elongation factor (a purified Leishmania protein; Corixa Corporation, Seattle, Wash.); ISCOMS (immunostimulating complexes which contain mixed saponins, lipids and form virus-sized particles with pores that can hold antigen; CSL, Melbourne, Australia); Pam3Cys; SB-AS4 (SmithKline Beecham adjuvant system #4 which contains alum and MPL; SBB, Belgium); non-ionic block copolymers that form micelles such as CRL 1005 (these contain a linear chain of hydrophobic polyoxypropylene flanked by chains of polyoxyethylene, Vaxcel, Inc., Norcross, Ga.); and Montanide IMS (e.g., IMS 1312, water-based nanoparticles combined with a soluble immunostimulant, Seppic).

Adjuvants may be TLR ligands. Adjuvants that act through TLR3 include without limitation double-stranded RNA. Adjuvants that act through TLR4 include without limitation derivatives of lipopolysaccharides such as monophosphoryl lipid A (MPLA; Ribi ImmunoChem Research, Inc., Hamilton, Mont.) and muramyl dipeptide (MDP; Ribi) andthreonyl-muramyl dipeptide (t-MDP; Ribi); OM-174 (a glucosamine disaccharide related to lipid A; OM Pharma SA, Meyrin, Switzerland). Adjuvants that act through TLRS include without limitation flagellin. Adjuvants that act through TLR7 and/or TLR8 include without limitation single-stranded RNA, oligoribonucleotides (ORN), synthetic low molecular weight compounds such as imidazoquinolinamines (e.g., imiquimod (R-837), resiquimod (R-848)). Adjuvants acting through TLR9 include without limitation DNA of viral or bacterial origin, or synthetic oligodeoxynucleotides (ODN), such as CpG ODN. Another adjuvant class is phosphorothioate containing molecules such as phosphorothioate nucleotide analogs and nucleic acids containing phosphorothioate backbone linkages.

The adjuvant can also be oil emulsions (e.g., Freund's adjuvant); saponin formulations; virosomes and viral-like particles; bacterial and microbial derivatives; immunostimulatory oligonucleotides; ADP-ribosylating toxins and detoxified derivatives; alum; BCG; mineral-containing compositions (e.g., mineral salts, such as aluminium salts and calcium salts, hydroxides, phosphates, sulfates, etc.); bioadhesives and/or mucoadhesives; microparticles; liposomes; polyoxyethylene ether and polyoxyethylene ester formulations; polyphosphazene; muramyl peptides; imidazoquinolone compounds; and surface active substances (e.g. lysolecithin, pluronic polyols, polyanions, peptides, oil emulsions, keyhole limpet hemocyanin, and dinitrophenol).

Adjuvants may also include immunomodulators such as cytokines, interleukins (e.g., IL-1, IL-2, IL-4, IL-5, IL-6, IL-7, IL-12, etc.), interferons (e.g., interferon-.gamma.), macrophage colony stimulating factor, and tumor necrosis factor.

In some embodiments, the adjuvant is a STING (STimulator of Interferon Genes) agonist. The STING signaling pathway in immune cells is a central mediator of innate immune response and when stimulated, induces expression of various interferons, cytokines and T cell recruitment factors that amplify and strengthen immune activity. Recent work has shown that STING agonists are effective adjuvants and efficiently elicit an immune response, described, for example in Dubensky, T., et al., Therapeutic Advances in Vaccines, Vol. 1(4): 131-143 (2013); and Hanson, M., et al., The Journal of Clinical Investigation, Vol. 125 (6): 2532-2546 (2015), the entire contents of each of which are hereby incorporated by reference.

In some embodiments, a STING agonist is a cyclic dinucleotide. In certain embodiments, cyclic dinucleotides include, but are not limited to, cdAMP, cdGMP, cdIMP, c-AMP-GMP, c-AMP-IMP, and c-GMP-IMP, and analogs thereof including, but not limited to, phosphorothioate analogues. In some embodiments, suitable cyclic dinucleotides for use in the present disclosure are described in some detail in, e.g., U.S. Pat. Nos. 7,709,458 and 7,592,326; WO 2007/054279; US 2014/0205653; and Yan et al. Bioorg. Med. Chem Lett. 18: 5631 (2008), each of which is hereby incorporated by reference.

In certain embodiments, a STING agonist is chemically synthesized. In certain embodiments, a STING agonist is an analog of a naturally occurring cyclic dinucleotide. STING agonists, including analogs of cyclic dinucleotides, suitable for use in the disclosure are provided in U.S. Pat. Nos. 7,709,458 and 7,592,326; and US 2014/0205653.

In some embodiments, the adjuvant is saponin monophosphoryl-lipid-A (MPLA) nanoparticle adjuvant (SMNP). In some embodiments, the adjuvant is cdGMP.

### (iii) Transmucosal Administration

The present disclosure provides methods of vaccinating and/or immunizing a subject comprising transmucosally (e.g., intranasally) administering the vaccine in an effective amount to the subject. Transmucosal administration includes nasal, oral (sublingual), intratracheal, vaginal and rectal routes. Transmucosal administration is sometimes preferred to parenteral routes of administration (e.g., subcutaneous, instramuscular, intravenous and intrathecal) because it is non-invasive, does not required trained medical personnel to administer, and is possible for a subject to self-administer.

In some embodiments, e.g., wherein the immunogen is a peptide antigen, the transmucosal administration does not include intratracheal administration.

In some embodiments, the present disclosure provides methods of vaccinating a subject comprising intranasally administering the vaccine in an effective amount to the subject. In some embodiments, the present disclosure provides methods of immunizing a subject comprising intranasally administering the vaccine in an effective amount to the subject.

In some embodiments, the subject is a mammal. In some embodiments, the subject is a non-human mammal, or a primate. In some embodiments, the subject is human.

In some embodiments, the vaccine is administered repeatedly. In certain embodiments, an initial dose may be followed by administration of a second or a plurality of subsequent doses of the vaccine in an amount that can be approximately the same or less or more than that of the initial dose. In some embodiments, at least 2 doses, at least 3 doses, at least 4 doses, or at least 5 doses of the vaccine are administered to elicit an effective immune response (e.g., inducing an antibody-mediated immune response, inducing a cell-mediated immune response, and/or achieving a desired level of neutralizing antibodies).

In some embodiments, a subsequent dose of the vaccine is administered about 1 week, 2 weeks, 3 weeks, a month, 1.5 months, 2 months, 2.5 months, 3 months, 4 months, 5 months, 6 months, 9 months, or a year or more after administration of a previous dose.

In some embodiments, the vaccine is administered every 2 weeks, every 4 weeks, every 6 weeks, every 8 weeks, every 10 weeks, every 12 weeks, or every 16 weeks.

In some embodiments, a booster dose of the vaccine is administered one to several years (e.g., 2 years, 3 years, 5 years, 10 years, 15 years) after a previous dose.

In some embodiments, a dose of the vaccine comprises about 1 to 500 μg, 20 to 500 μg, 50 to 450 μg, 75 to 400 μg, 100 to 300 μg, or 150 to 250 μg of the amphiphilic conjugate. In some embodiments, a dose of the vaccine comprises about 1 μg, 2 μg, 3 μg, 4 μg, 5 μg, 6 μg, 7 μg, 8 μg, 9 μg, 10 μg, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg, 200 μg, 210 μg, 220 μg, 230 μg, 240 μg, 250 μg, 260 μg, 270 μg, 280 μg, 290 μg, or 300 μg of the amphiphilic conjugate.

In some embodiments, the vaccine is administered in combination with an SMNP adjuvant. In some embodiments, an amount of about 1 to 400 μg, 1 to 50 μg, 50 to 100 μg, 50 to 200 μg, 50 to 300 μg, 50 to 400 μg, 100 to 200 μg, 100 to 300 μg, 100 to 400 μg, 200 to 400 μg, or 300 to 400 μg of SMNP is administered in combination with a dose of the vaccine. In some embodiments, about 1 μg, 2 μg, 3 μg, 4 μg, 5 μg, 6 μg, 7 μg, 8 μg, 9 μg, 10 μg, 20 μg, 30 μg, 40 μg, 50 μg, 60 μg, 70 μg, 80 μg, 90 μg, 100 μg, 125 μg, 150 μg, 175 μg, 200 μg, 225 μg, 250 μg, 275 μg, 300 μg, 325 μg, 350 μg, 375 μg, 400 μg of SMNP is administered in combination with a dose of the vaccine.

In some embodiments, the vaccine is administered in combination with an cdGMP adjuvant. In some embodiments, an amount of about 5 to 50 μg, 50 to 150, or 100 to 400 μg of cdGMP is administered in combination with a dose of the vaccine. In some embodiments, about 5 μg, 10 μg, 15 μg, 20 μg, 25 μg, 30 μg, 40 μg, 50 μg, 60 μg, 70 μg, 80 μg, 90 μg, 100 μg, 125 μg, 150 μg, 175 μg, 200 μg, 225 μg, 250 μg, 275 μg, 300 μg, 325 μg, 350 μg, 375 μg, 400 μg of cdGMP is administered in combination with a dose of the vaccine.

### (iv) Target Infectious Diseases

In certain aspects, the methods provided herein comprise inducing an immune response to prevent or reduce severity of an infectious disease.

In some embodiments, the infectious disease is caused by a pathogen. In some embodiments, the pathogen can infects a subject through mucosal surfaces.

Infectious disease that can benefit from the methods provided herein include, but are not limited to, HIV/AIDS, coronavirus disease 19 (COVID-19), influenza, rotavirus infection (e.g., diarrhea), cytomegalovirus (CMV) infection, Epstein-Barr virus infection (e.g., mononucleosis), respiratory syncytial virus (RSV) infection, and cholera.

Acquired immunodeficiency syndrome (AIDS) is a syndrome that is caused by human immunodeficiency virus (HIV). HIV is spread primarily by unprotected sex (including anal and vaginal sex), contaminated hypodermic needles or blood transfusions, and from mother to child during pregnancy, delivery, or breastfeeding. Following initial infection of HIV, an individual may not notice any symptoms, or may experience a brief period of influenza-like illness. Typically, this is followed by a prolonged incubation period with no symptoms. If the infection progresses, it interferes with the immune system, increasing the risk of developing common infections such as tuberculosis, as well as other opportunistic infections, and tumors which are rare in people who have normal immune function. These late symptoms of infection are referred to as acquired immunodeficiency syndrome (AIDS).

Coronavirus disease 19 (COVID-19) is a respiratory disease caused by the SARS-CoV-2 virus, a member of a large family of viruses called coronaviruses. The virus is thought to spread from person to person through droplets released when an infected person coughs, sneezes, or talks. It may also be spread by touching a surface with the virus on it and then touching one's mouth, nose, or eyes, though less common.

Influenza (also known as “flu”) is an infection of the nose, throat and lungs caused by influenza virus. There are four types of influenza virus, termed influenza viruses A, B, C, and D. Aquatic birds are the primary source of Influenza A virus (IAV), which is also widespread in various mammals, including humans and pigs. Influenza B virus (IBV) and Influenza C virus (ICV) primarily infect humans, and Influenza D virus (IDV) is found in cattle and pigs. IAV and IBV circulate in humans and cause seasonal epidemics, and ICV causes a mild infection, primarily in children. In humans, influenza viruses are primarily transmitted through respiratory droplets produced from coughing and sneezing. Transmission through aerosols and intermediate objects and surfaces contaminated by the virus also occur.

Rotarovirus infection commonly results in severe, watery diarrhea and vomiting in infants and young children, which could lead to hospitalization and death in children. People who are infected with rotavirus shed the virus in their stool, and rotarovirus spreads via fecal-oral transmission.

Cytomegalovirus (CMV) infection is a common infection that infects people of all ages. Most people infected with CMV show no signs or symptoms, and the virus can be dormant (inactive) in various tissues for a long time. Various stimuli can reactivate the dormant CMV, resulting in virus growth which can sometimes cause disease. Serious infections typically develop only in infants infected before birth and in people with a weakened immune system. Infected people may shed CMV in their urine or saliva intermittently. The virus is also excreted in mucus in the cervix (the lower part of the uterus), semen, stool, and breast milk. Thus, the virus is spread through sexual and nonsexual contact.

Epstein-Barr virus (EBV, also known as human herpesvirus 4) is the virus that infects B cells, with infection ranging from asymptomatic to infectious mononucleosis. EBV spreads most commonly through bodily fluids, especially saliva. However, EBV can also spread through blood and semen during sexual contact, blood transfusions, and organ transplantations.

Respiratory syncytial virus (RSV) is a respiratory virus that infects lungs and breathing passages. In adults and older, healthy children, RSV symptoms are mild and typically mimic the common cold. However, in young children, older adults, people with heart and lung disease, or anyone with a weak immune system, RSV infection can be severed. RSV is spread through contact with droplets from the nose and throat of infected people when they cough and sneeze. RSV can also spread through dried respiratory secretions on bedclothes and similar items.

Cholera is an acute diarrheal illness caused by infection of the intestine with Vibrio cholerae bacteria. People can get sick when they swallow food or water contaminated with cholera bacteria. The infection is often mild or without symptoms, but can sometimes be severe and life-threatening.

In certain aspects, the methods provided herein comprise inducing immunity to an infectious pathogen. Non-limiting examples of the infectious pathogen include a human immunodeficiency virus (HIV), a SARS-CoV-2 virus, an influenza virus, a rotavirus, a cytomegalovirus (CMV), an Epstein-Barr virus (EBV), a respiratory syncytial virus (RSV), and a cholera bacteria. Immunity against other common infectious pathogens can also be induced using the methods described herein.

In some embodiments of the methods provided herein, the immune response that is induced in the subject comprises expression of an IgA antibody targeting the pathogen. In some embodiments, the immune response that is induced in the subject comprises expression of IgG antibodies targeting the pathogen. In some embodiments, the immune response that is induced in the subject comprises expression of both IgA and IgG antibodies targeting the pathogen. In some embodiments, the immune response that is induced in the subject comprises expression of neutralizing antibodies targeting the pathogen.

### EXAMPLES

This invention is further illustrated by the following examples which should not be construed as limiting. The contents of all references, GenBank Accession and Gene numbers, and published patents and patent applications cited throughout the application are hereby incorporated by reference. Those skilled in the art will recognize that the invention may be practiced with variations on the disclosed structures, materials, compositions and methods, and such variations are regarded as within the ambit of the invention.

Reference numbers in brackets “[ ]” herein refer to the corresponding literature listed in the attached Bibliography which forms a part of this Specification, and the literature is incorporated by reference herein.

### Example 1: Summary

A vaccine platform that uses endogenous albumin as a chaperone to enhance lymph node trafficking of peptide antigens or molecular adjuvants following parenteral injection was previously developed. One of albumin's primary functions in vivo is to serve as a fatty acid transporter, as albumin bears seven different lipid binding pockets [23, 24]. By conjugating peptides or Toll-like receptor agonist adjuvants to an amphiphilic albumin-binding lipid tail (forming an ‘amph-vaccine’), important changes to the pharmacokinetic behavior of these vaccine components can be achieved: First, following injection, the lipid tail of amph-vaccines associates with endogenous albumin present in the interstitial fluid at the injection site, causing the conjugates to be efficiently redirected to lymphatic vessels and draining lymph nodes, following the convection path of albumin (whereas unmodified peptides disperse into the blood where they are rapidly diluted and degraded) [25]. Second, upon reaching the dense cellular microenvironment of lymph nodes, the lipid tails of amph-peptides insert into cell membranes, promoting prolonged antigen retention in the draining lymphoid tissue [26, 27]. These alterations in pharmacokinetics of amph-peptides compared to soluble peptide vaccines lead to strong enhancements in systemic T cell responses and anti-tumor immunity following parenteral immunization [25, 28, 29].

In addition to constitutive trafficking from blood to tissues to lymph, albumin is also bidirectionally transported across mucosal barriers via interactions with the neonatal Fc receptor (FcRn) expressed by mucosal epithelial cells. The FcRn has received attention as a ‘mucosal gateway’ for improving drug uptake across the mucosal epithelium in nasopharyngeal, pulmonary, and gastrointestinal tissues [7, 30-32]. It is widely expressed on mucosal epithelial cells in adult animals and humans, where it plays an essential role in recycling IgG and albumin through bidirectional transcytosis of both molecules [33-35]. Albumin-binding amph-vaccines might be capable of FcRn-mediated uptake across the mucosa, e.g., nasal mucosa, enabling higher levels of antigen to reach the NALT. In addition, membrane tethering of amph-immunogens might prolong the availability of antigen in the nasal passages and NALT tissue, to promote local immune priming while avoiding systemic dissemination of antigen away from the site of action of locally co-administered mucosal adjuvants. It was hypothesized that together these two effects may promote stronger mucosal and systemic immunity.

Given that the majority of licensed vaccines are thought to operate via induction of protective antibody responses [36,37], the examples provided herein prepared and tested large protein immunogen amphiphilic conjugates designed to elicit humoral immune responses in the setting of HIV and SARS-CoV-2. As described below, the amphiphilic conjugates showed enhanced persistence and uptake across the nasal mucosa compared to unmodified antigens, leading to greatly increased GC and follicular helper T cell (Tfh) responses in the NALT. Intranasal amphiphilic conjugate immunization led to high levels of IgG and IgA in serum, upper and lower respiratory mucosa, and distal genitourinary mucosal sites, including the induction of substantial neutralizing antibody responses to a SARS-Cov-2 RBD immunogen. Further, amphiphilic conjugate immunization enhanced vaccine uptake in the nasal passages of non-human primates and enhanced IgG and IgA responses relative to soluble protein immunization. Together, the data presented in the Examples herein demonstrate that vaccines of the present disclosure enhance both mucosal and systemic immunity elicited by intranasal immunization.

### Example 2: Synthesis of Protein Antigen-Amphiphile Conjugates with Albumin Binding and Membrane Insertion Properties

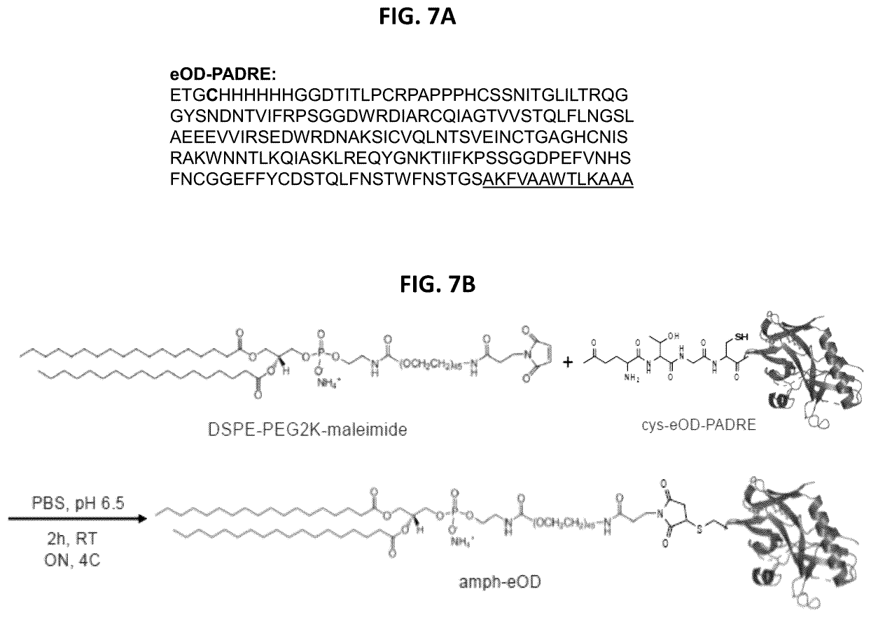

To assess whether appending an albumin-binding moiety to subunit protein vaccine antigens could alter antigen uptake across the nasal mucosa, conjugates of an HIV Env protein immunogen linked to a poly(ethylene glycol) (PEG)-DSPE amphiphile were first synthesized. This PEG-lipid was previously demonstrated to bind to albumin with an equilibrium K_{D}˜125 nM [25]. As a test antigen for this concept, the Env immunogen eOD-GT8 (gp120 engineered outer domain-germline targeting immunogen 8, hereafter eOD), a ˜25 kDa germline targeting antigen that was recently shown to successfully prime VRC01-class HIV broadly neutralizing antibody responses in a phase I clinical trial [38-41] was selected. eOD was fused at the C-terminus with the PADRE universal helper epitope and a terminal free cysteine was introduced at the N-terminus to enable coupling to maleimide-functionalized PEG2K-DSPE to form a thioether linkage (FIGS. 7A-7B). The resulting amph-eOD (FIG. 1A) formed ˜30 nm diam. micelles in aqueous solution (FIG. 1B), facilitating purification from unreacted eOD (˜5 nm) by size exclusion chromatography (SEC) (FIG. 1C).

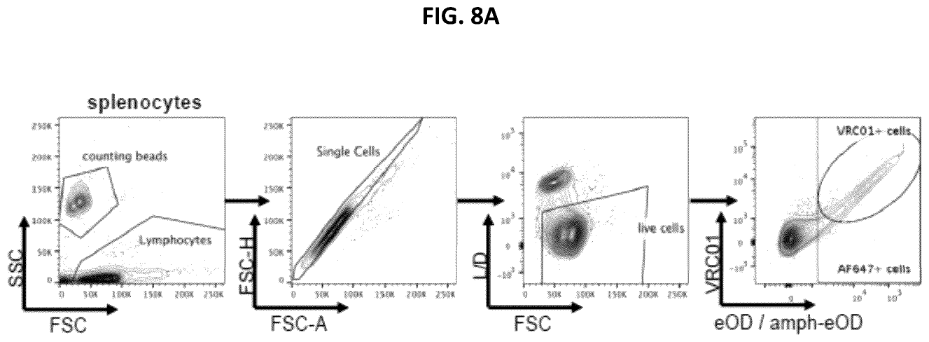

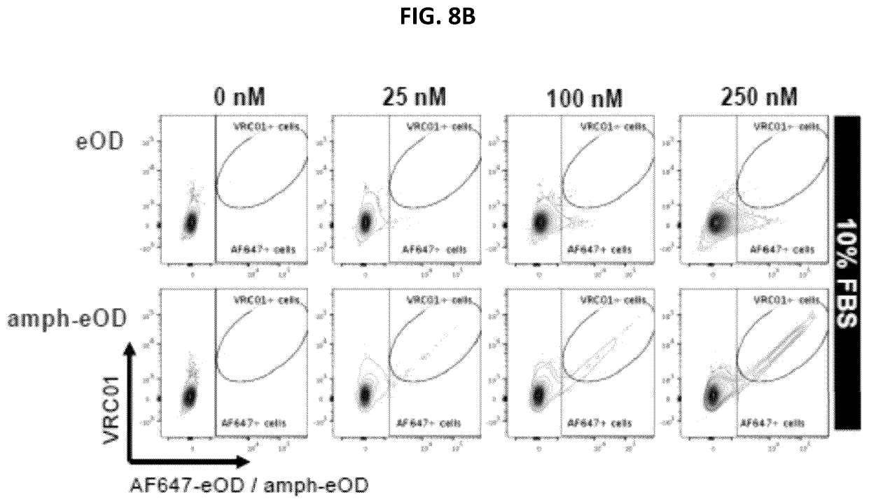

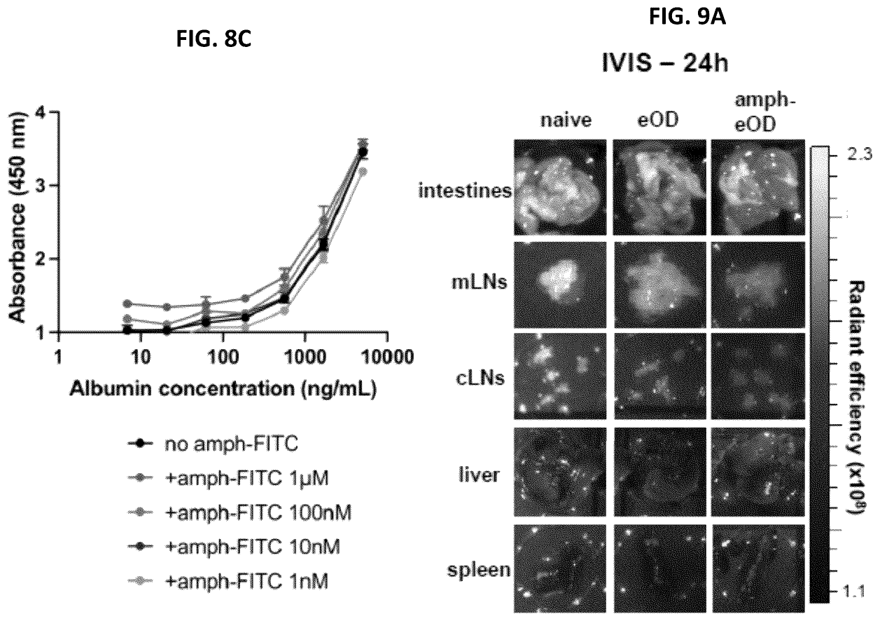

It was previously shown that PEG-DSPE coupling to small peptide antigens endows the conjugates with the ability to bind to albumin, and to also interact with cell membranes, altering in vivo trafficking behavior [25, 26]. To evaluate whether the amphiphile tail could similarly alter the behavior of much larger protein immunogens, fluorescently-labeled amph-eOD was first incubated with an albumin-functionalized agarose resin for 2 hr at 37° C. followed by separation of the resin and measurement of protein remaining in solution. Sixty percent of added amph-eOD bound to the albumin-resin, versus <5% of unmodified eOD (FIG. 1D). Next, the interaction of amph-eOD with lymphocytes was assessed. Titrated concentrations of Alexa dye-labeled eOD or amph-eOD were added to mouse splenocytes in 10% serum at 37° C., then stained extracellularly at 4° C. with fluorescently-labeled VRC01 monoclonal antibody to detect eOD coating the cell surfaces. Flow cytometry analysis revealed that both eOD and amph-eOD showed association with splenocytes within 1 hr, but amph-eOD showed >15-fold greater levels of uptake (FIGS. 1E-1G, FIGS. 8A-8B). Further, the vast majority of cell-associated amph-eOD was localized on the cell surfaces, as revealed by VRC01 staining (FIGS. 1E-1G). The percentage of eOD+VRC01+ double positive cells increased proportionally with amph-eOD but not eOD concentration (FIGS. 1F-1G). Thus, amph-protein conjugates were surprisingly found to exhibit albumin-binding and membrane-insertion properties similar to previously studied amph-peptide conjugates, which we hypothesized would alter antigen trafficking and persistence in vivo.

### Example 3: Amphiphile Modification Enhances Uptake and Retention of eOD Antigen in the Nasal Cavity Following Intranasal Immunization in Mice

Albumin is transported bi-directionally across respiratory mucosal surfaces via interactions with the neonatal Fc receptor (FcRn) [31, 42, 43]. Amph-protein immunogens might show enhanced uptake across the nasal mucosal epithelium by using albumin as a non-covalent chaperone. To test this idea, it was first assessed whether DSPE-PEG binding to albumin would inhibit its interaction with FcRn using an enzyme-linked immunosorbent assay (ELISA) to measure albumin binding to plate-bound FcRn. Incubation of albumin with fluorescein isothiocyanate-labeled DSPE-PEG at concentrations up to 1 μM showed no inhibition of albumin-FcRn binding (FIG. 8C).

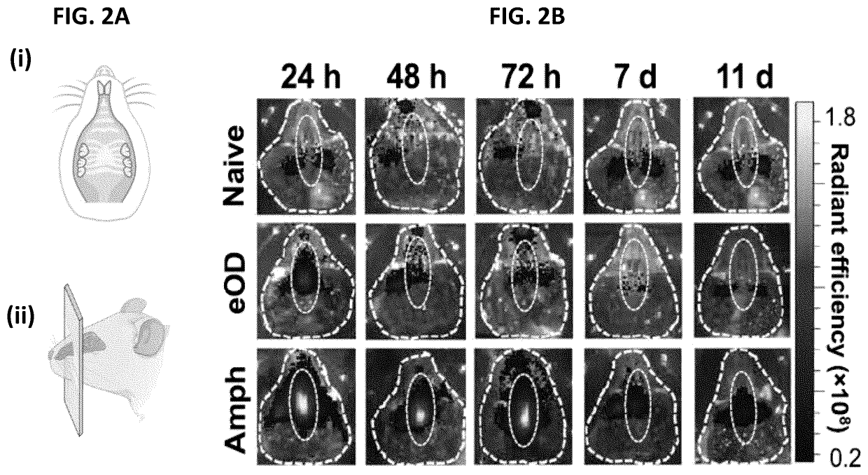

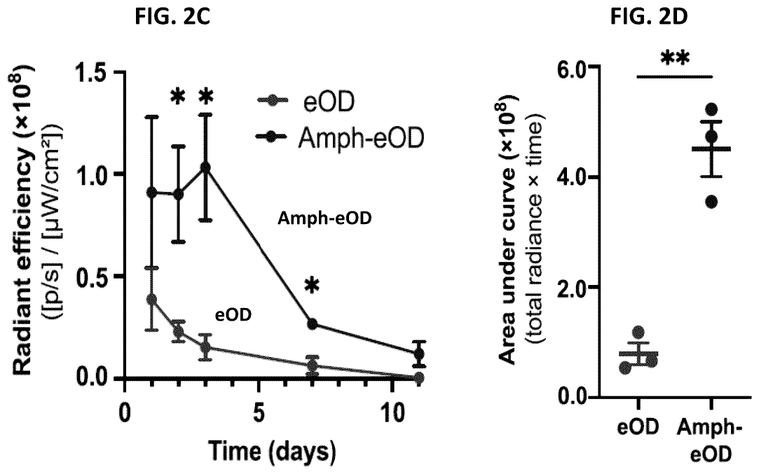

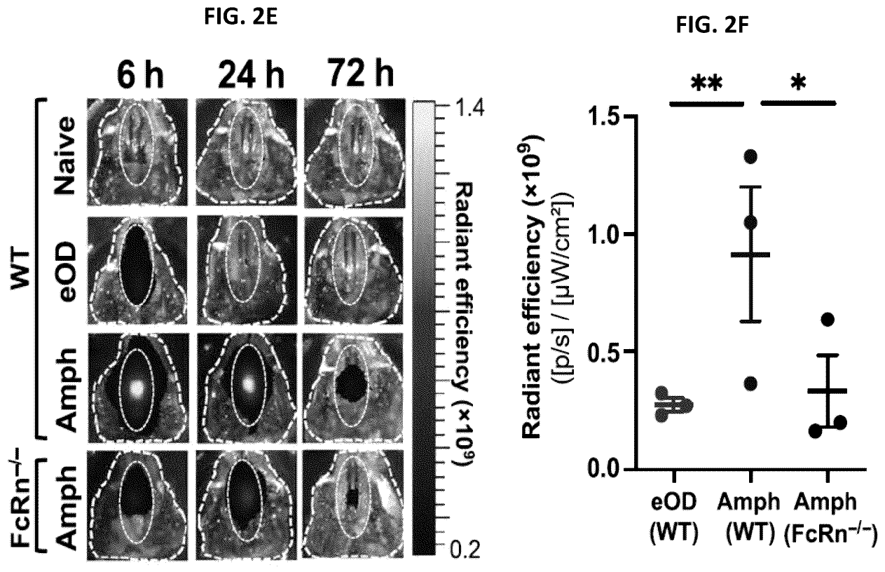

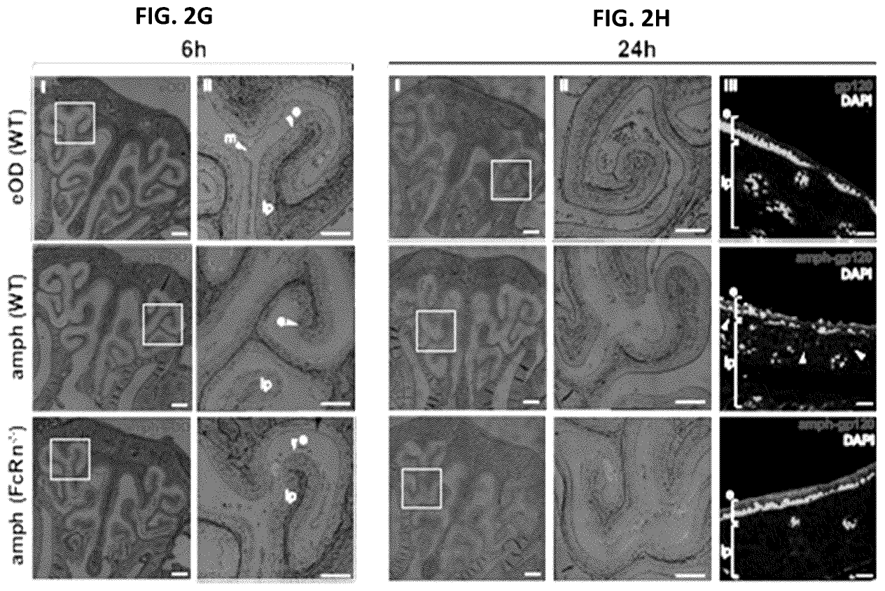

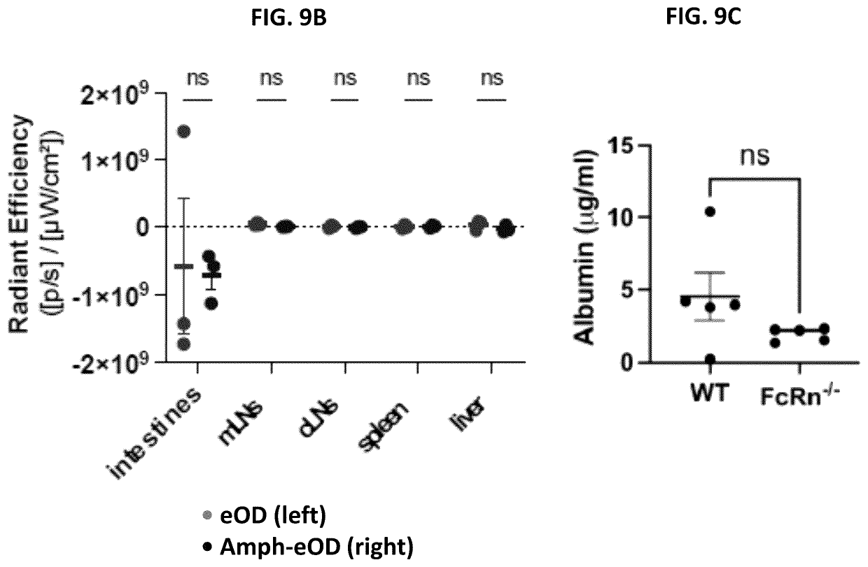

Trafficking of fluorescent amph-eOD vaccine in the nasal cavity of mice over time following intranasal administration was next investigated. Total vaccine uptake in the nasal cavity was quantified by In Vivo Imaging System (IVIS) measurement of fluorescence signal in a defined region of interest (ROI) of the mouse snout over time (FIG. 2A, (i)) and was further characterized by histological imaging of cross-sections of the nasal cavity (FIG. 2A, (ii)). First, BALB/c mice were immunized intranasally with Alexa fluor-labeled eOD or amph-eOD mixed with saponin adjuvant; upper jaws were removed from the mouse snout and the signal on the ventral side of the nasal cavity was quantified by IVIS over 11 days (FIG. 2B). Amph-eOD showed significant accumulation and persistence in the nasal cavity over 72 h, with vaccine still detectable at 7- and 11-days post-immunization (FIG. 2B-C). By contrast, free eOD exhibited some initial signal at 24 h (<40% of amph-eOD), which quickly decreased to background. Vaccine exposure assessed as area-under the-curve (AUC) for the nasal fluorescence signal over time was ˜5.7× greater for amph-eOD than eOD (FIG. 2D). Furthermore, amph-eOD did not disseminate to reach the systemic compartment or distal lymphatic tissues, as negligible vaccine accumulation was observed by IVIS in the spleen, liver, intestines, cervical LNs, or mesenteric LNs at 24 h (FIGS. 9A-9B).

While not wishing to be bound by theory, enhanced amph-vaccine persistence in the nasal cavity could be mediated by a combination of (1) the lipid tail promoting association with the epithelial cell surfaces and (2) amphiphile binding to albumin in the mucus layer promoting FcRn-mediated transcytosis into the underlying nasal submucosa. Notably, IVIS imaging revealed rapid clearance of amph-eOD administered with adjuvant i.n. in FcRn^{-/-} mice compared to wild type (WT) animals; amph-eOD persistence in the FcRn-deficient animals was similar to unmodified eOD in WT mice (FIGS. 2E-2F).

To determine whether enhanced antigen persistence correlated with actual uptake into the nasal tissue, histological sections from the mid-point of the nasal passages were imaged (FIG. 2A, (ii)). Confocal imaging revealed immediate qualitative differences in vaccine accumulation and uptake in the nasal cavity at 6 h (FIG. 2G). eOD was only faintly observed on the epithelial cell surface (‘e’) and appeared instead to be primarily trapped at the top of the mucus layers (‘m’) lining the airways (FIG. 2G, (ii) right panels)). Conversely, amph-eOD was predominantly accumulated at the epithelial surface overlying the lamina propria (‘lp’) in WT mice, concentrating in the respiratory nasoturbinates. Amph-eOD also exhibited clear accumulation at the epithelial surface of FcRn^{-/-} mice (FIG. 2G, (i, ii)), which is distributed to the amphiphile tail's ability to insert into cell membranes. At 24 h post administration, eOD was nearly undetectable in the nasal cavity, while amph-eOD was still accumulated at the epithelial surface in WT and FcRn^{-/-} animals (FIG. 2H, (i, ii) left and middle panels)). However, higher magnification imaging with DAPI staining to delineate the epithelium and underlying submucosa revealed clear pockets of amph-eOD uptake into the lamina propria in WT mice; this submucosal accumulation was absent in FcRn^{-/-} mice (FIG. 2H, (iii) right panels). These data suggest that association of eOD with epithelial cells is promoted by the DSPE lipid tail, but transport across the epithelial barrier is significantly dependent on FcRn.

### Example 4: Intranasal Amph-gp120 Induces Superior Germinal Center and Tfh Cell Responses in NALT in an FcRn-Dependent Manner

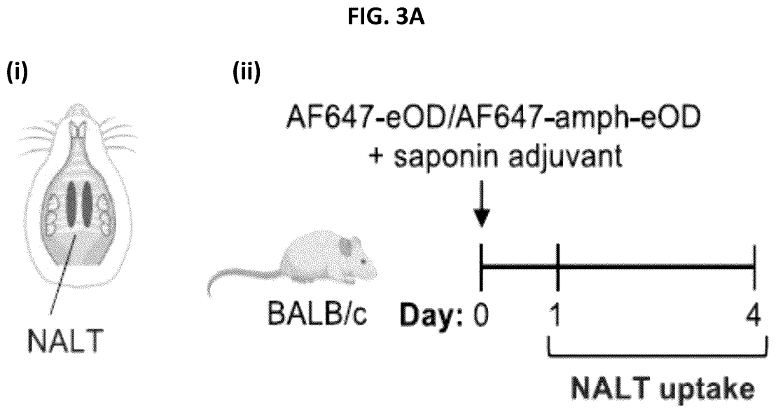

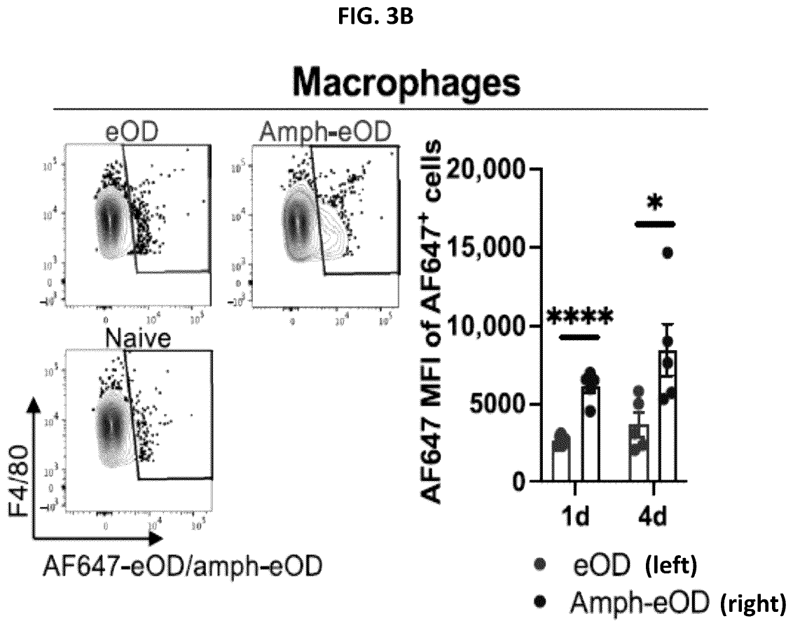

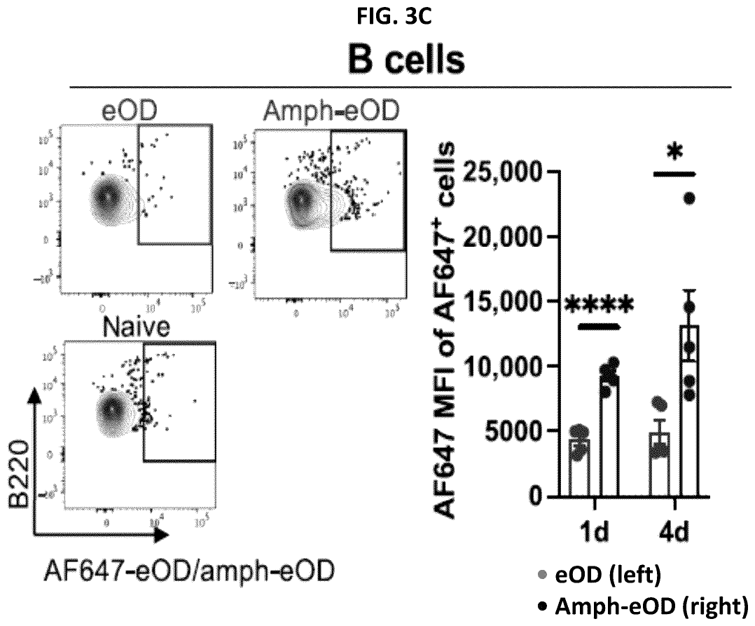

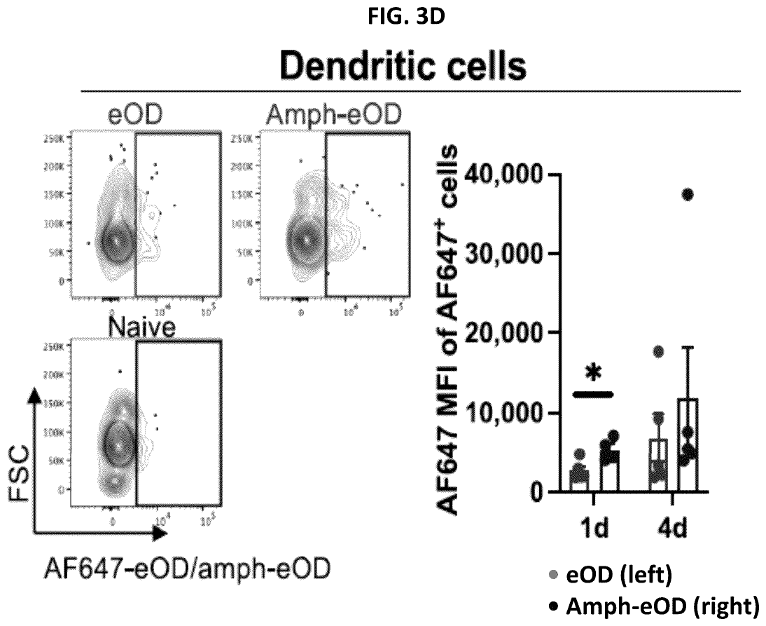

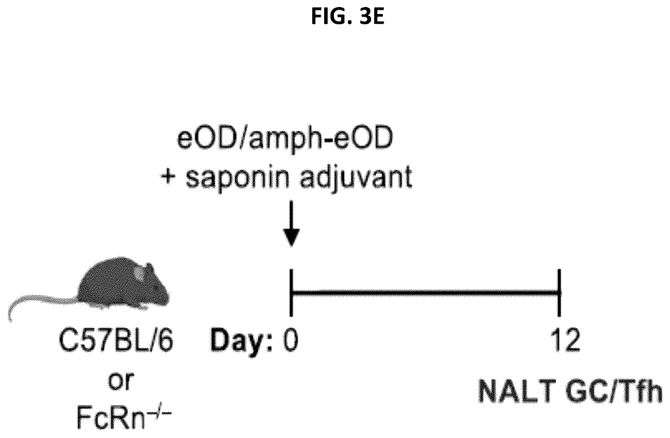

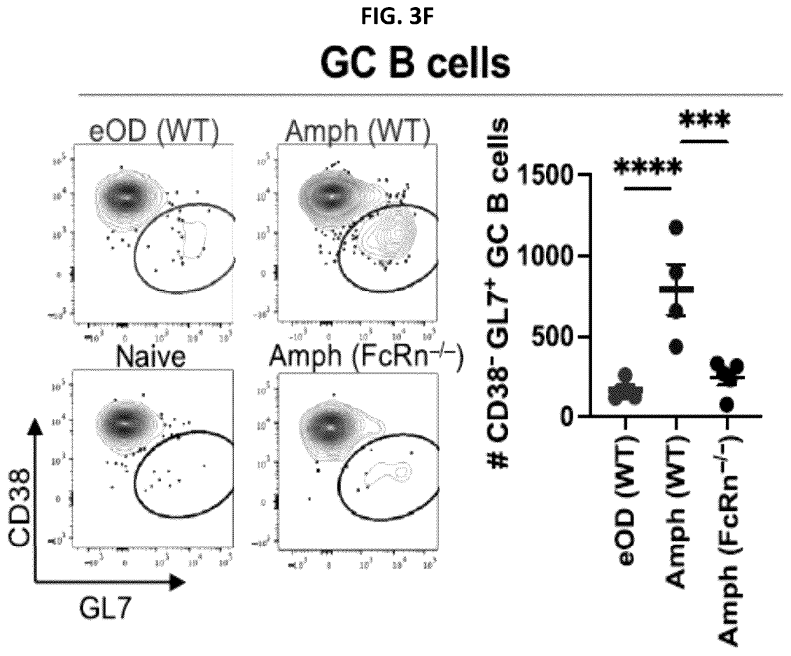

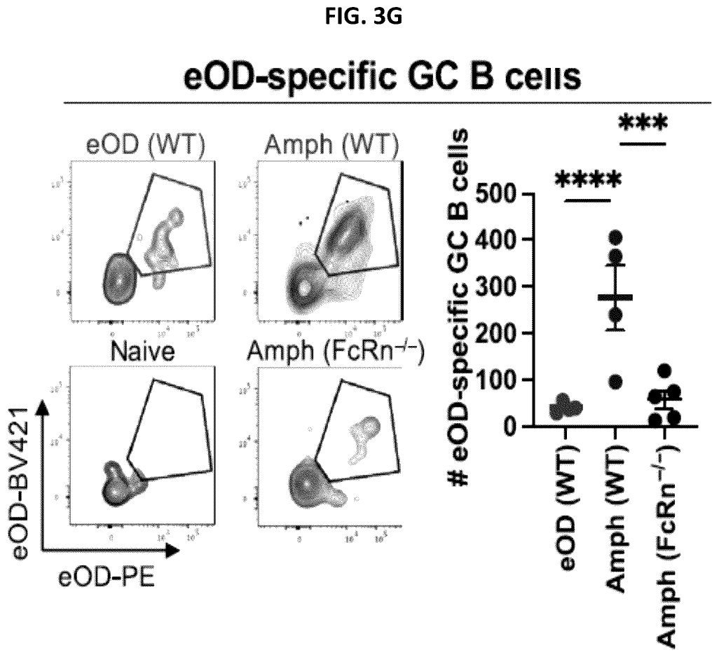

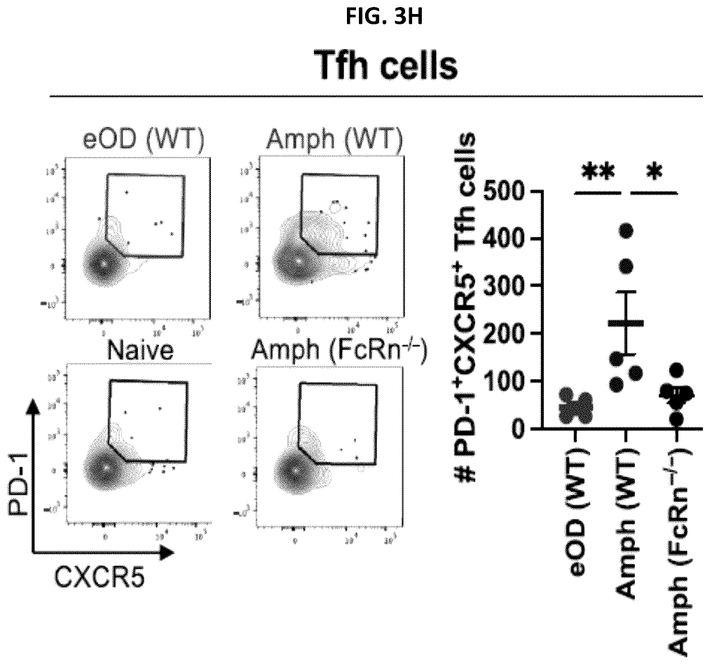

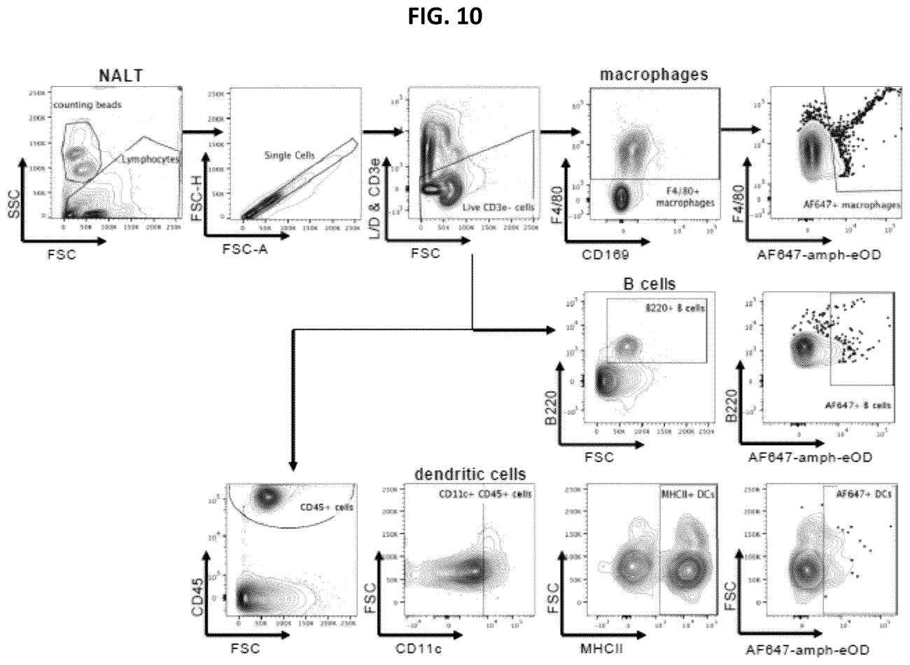

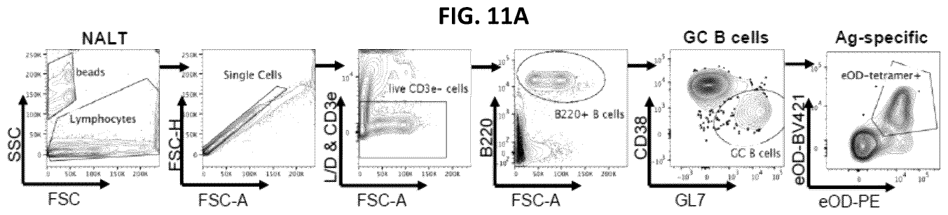

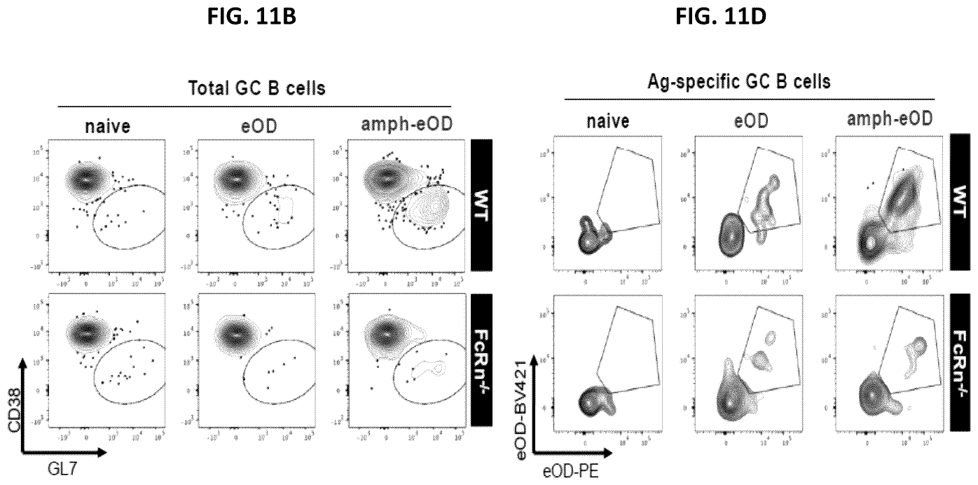

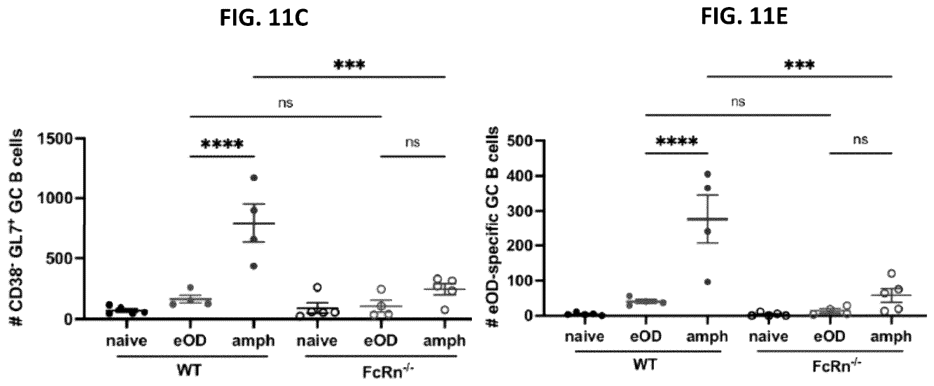

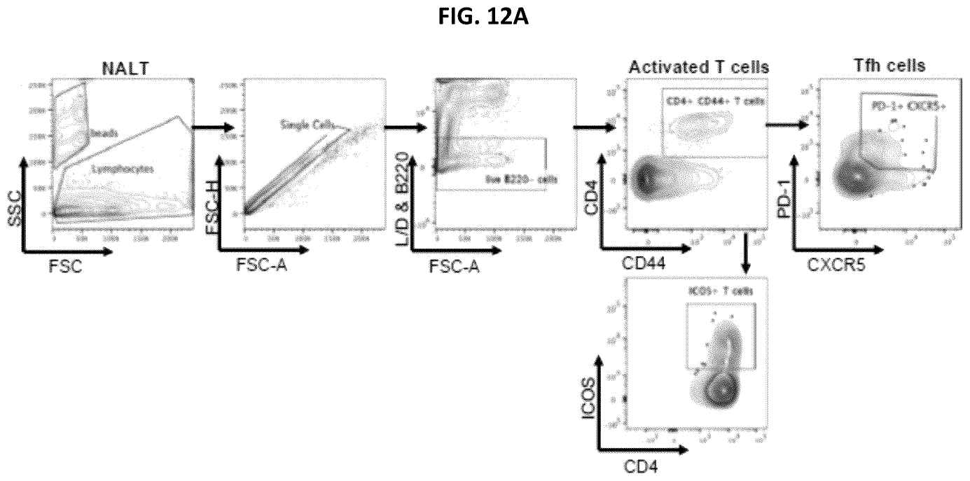

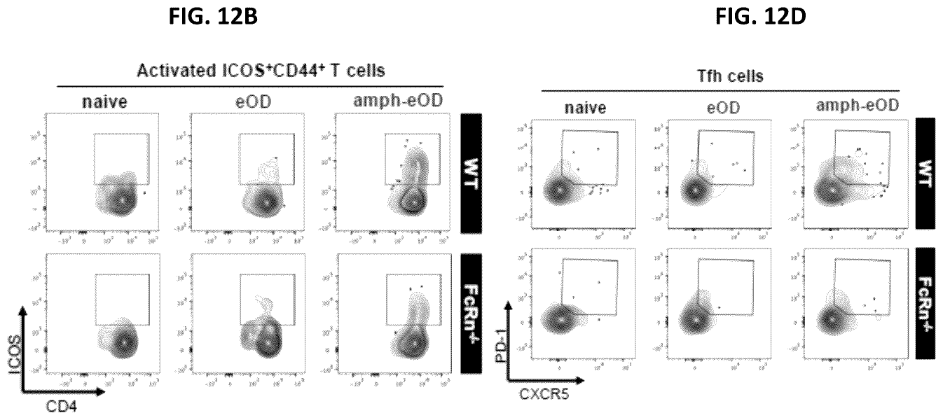

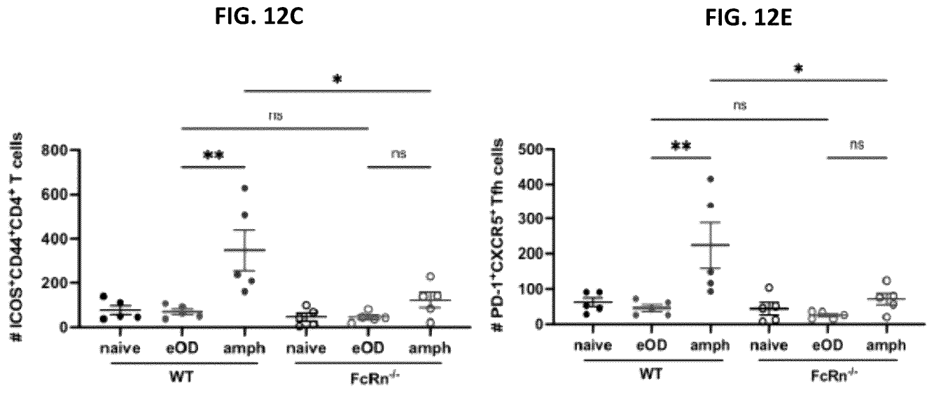

It was hypothesized that enhanced vaccine retention in the nasal cavity and increased uptake across the nasal mucosal epithelia would result in greater amounts of antigen reaching the NALT located on the dorsal side of the soft palate underlying the nasal passage (FIG. 3A, (i)), thereby priming a stronger local GC response. Thus, fluorescent eOD or amph-eOD accumulation and persistence in the NALT over time was investigated by flow cytometry following intranasal immunization (FIG. 3A, (ii)). Amph-eOD accumulation in F4/80⁺ macrophages and B cells significantly exceeded that of eOD both 1- and 4-days post-immunization (FIG. 3B-C, FIG. 10). Uptake in CD11c⁺MHCII⁺ dendritic cells was also greater for amph-eOD compared to eOD 1 day after immunization (FIG. 3D, FIG. 10). These findings indicate that amph-eOD reaches the NALT and is taken up by key antigen presenting cell (APC) populations to a greater extent than unmodified eOD. To determine the impact of enhanced antigen delivery to the nasal lymphoid tissue on the initial stages of the adaptive immune response to eOD, germinal center (GC) B cell and T follicular helper (Tfh) cell responses in the NALT were evaluated 12 days following i.n. immunization with eOD and saponin adjuvant (FIG. 3E). Amph-eOD induced a greater GC response in the NALT of WT mice, both in terms of total GC B cells (4.8-fold) and eOD-binding antigen-specific GC B cells (6.8-fold) in comparison to soluble eOD immunization (FIG. 3F-G, FIG. 11). Strikingly, these amplified responses were completely dependent on FcRn, as amph-eOD immunization in FcRn^{-/-} animals elicited responses comparable to eOD in WT mice (FIG. 3F-G, FIG. 11B-E). These trends were mirrored in NALT follicular helper T cell (Tfh) responses: amph-eOD elicited greater Tfh responses compared to both eOD in WT mice and amph-eOD in FcRn^{-/-} mice (FIG. 3H, FIG. 12A-E), and also induced greater overall activation of T cells (ICOS⁺CD4⁺CD44⁺ T cells) compared to eOD in WT mice (p<0.01) and amph-eOD in FcRn^{-/-} mice (p<0.05) (FIG. 12B-C). Thus, amph-conjugate immunization was found to greatly amplify mucosal GC and T cell responses in a manner dependent on FcRn.

### Example 5: Intranasal Amph-eOD Elicits Robust Systemic and Mucosal Antibody Responses in Mice

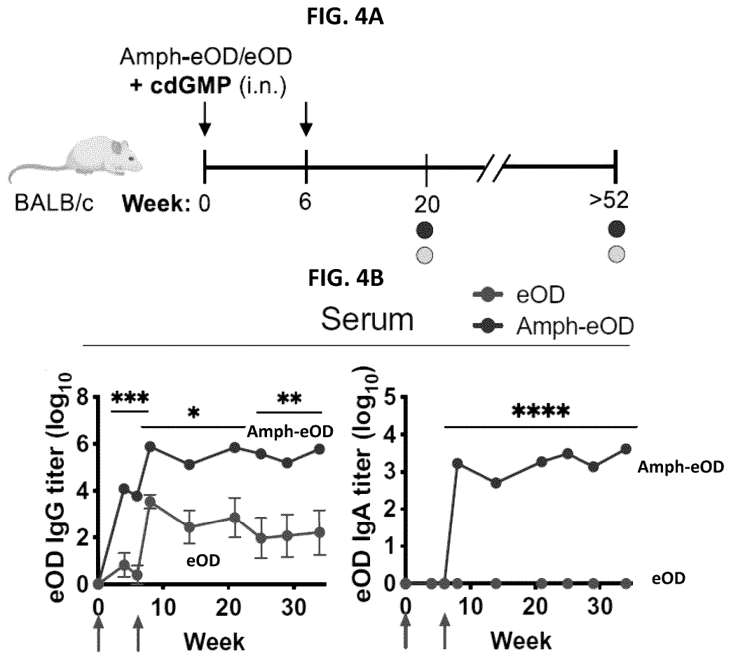

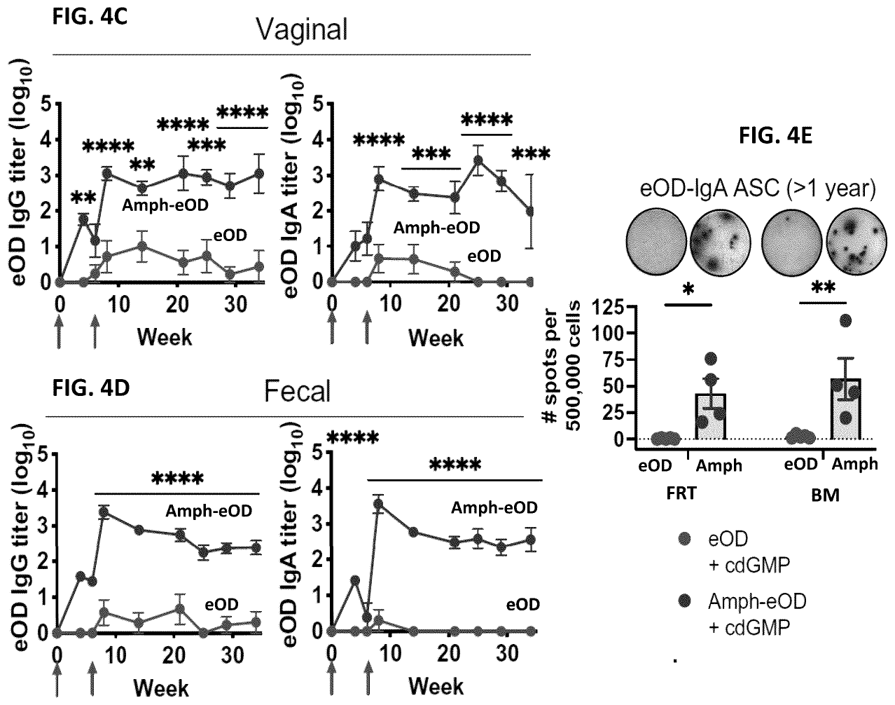

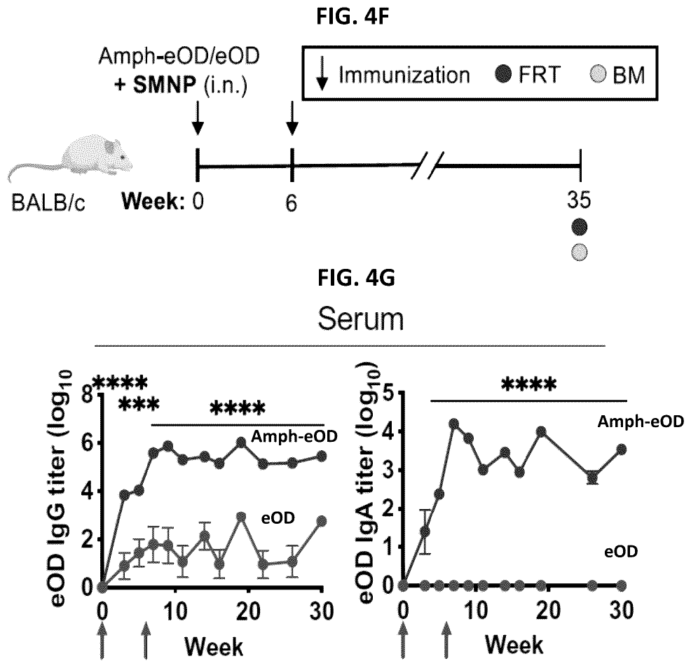

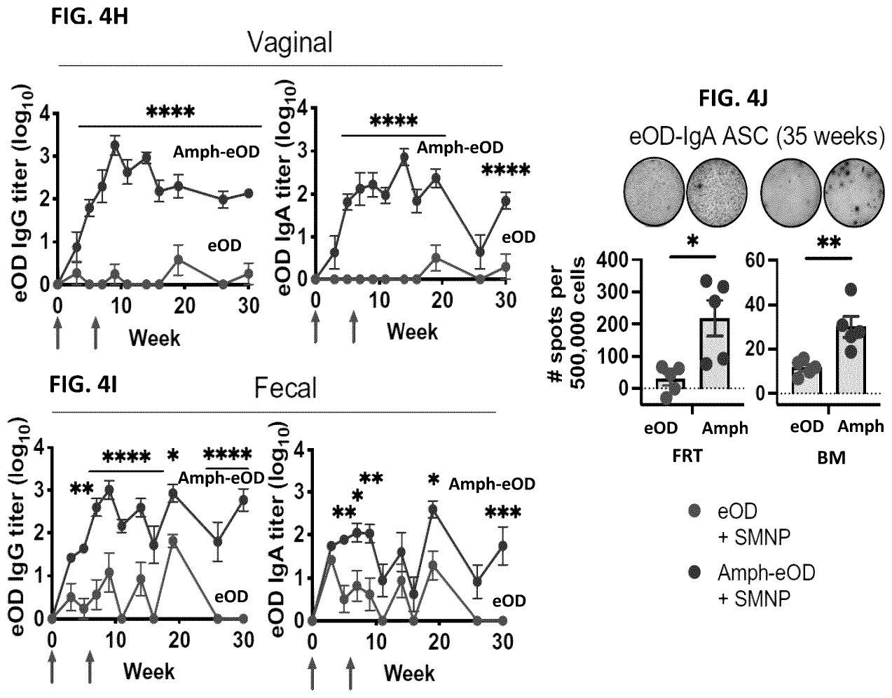

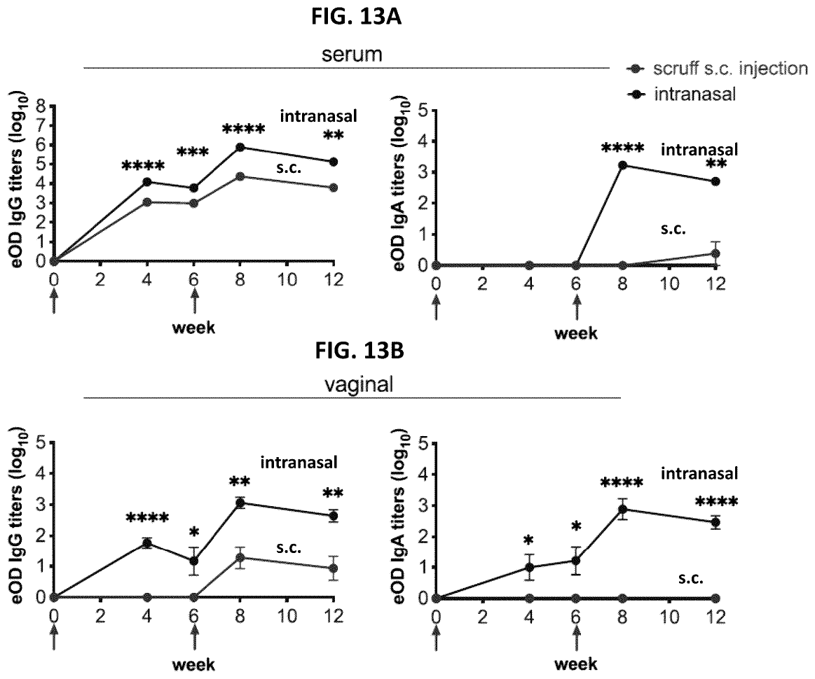

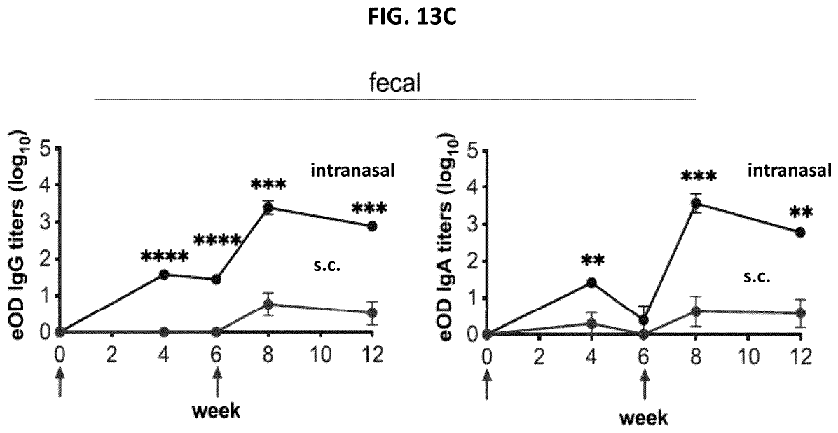

Output antibody responses elicited by i.n. amphiphile or soluble protein immunization were evaluated both systemically and at distal mucosal sites relevant for HIV transmission such as the rectal and genitourinary mucosa. First, studies combining eOD with the cyclic dinucleotide, cyclic dimeric guanosine monophosphate (cdGMP), were carried out (FIG. 4A). Cyclic dinucleotides (CDNs) activate the innate immune sensor STimulator of INterferon Genes (STING) and have been previously reported to be an effective mucosal vaccine adjuvant in mice [44-46]. Intranasal immunization with amph-eOD and cdGMP induced very high serum IgG and IgA responses, with endpoint antigen-specific serum IgG titers of ˜10⁶ and IgA titers of ˜10³-10⁴ that were sustained over 35 weeks (FIG. 4B). Amph-vaccination increased IgG responses over unmodified eOD by more than 2 logs, and primed strong serum IgA responses that were completely absent following soluble protein immunization. Notably, amph-eOD also induced striking sustained mucosal IgG and IgA responses in the vaginal tract (FIG. 4C) and rectal mucosa (FIG. 4D), where soluble eOD immunization again elicited only weak to undetectable responses. Intranasal versus parenteral (subcutaneous) vaccination with amph-eOD was also directly compared. Subcutaneous immunization with amph-eOD elicited potent systemic IgG titers in blood but failed to prime mucosal responses (FIGS. 13A-13C).

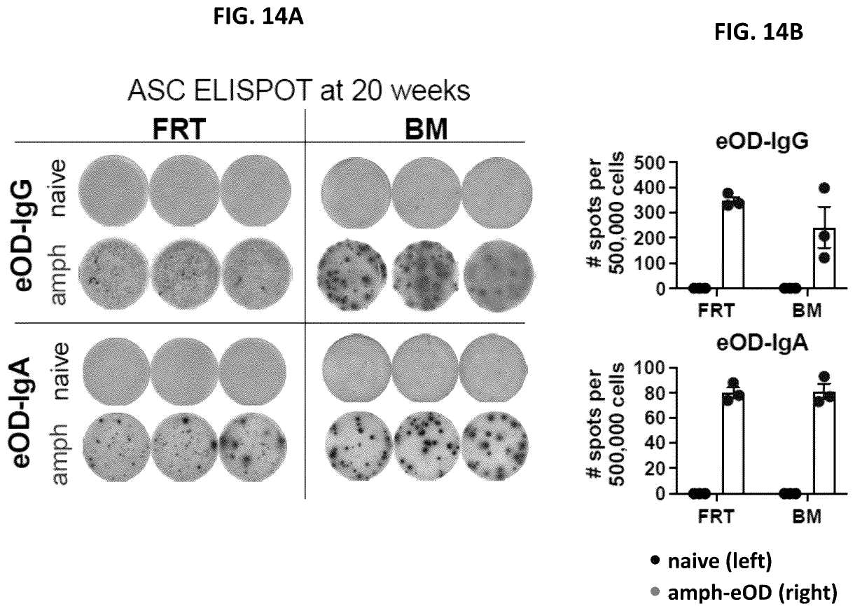

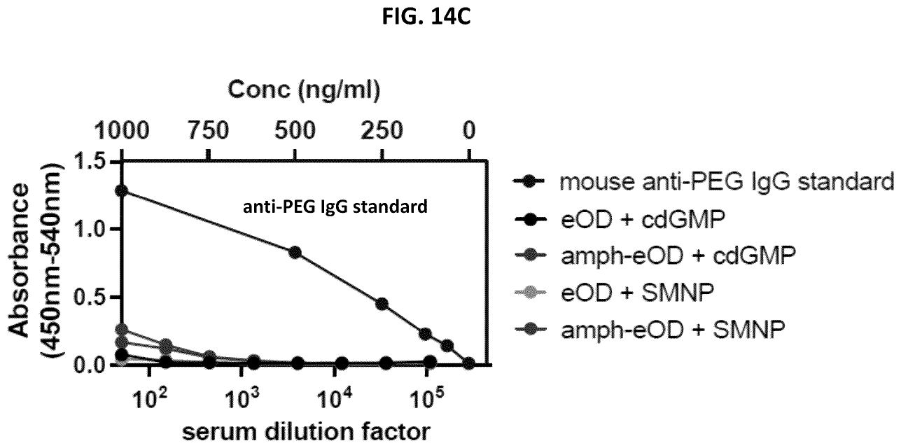

Next, cohorts of mice were euthanized at different time points and the female reproductive tract (FRT) and bone marrow (BM) were isolated and analyzed via antibody-secreting cell (ASC) ELISPOT to identify long-lived plasma cells. Amph-eOD immunization led to high levels of both eOD-specific IgA and IgG plasma cells in the FRT and BM 20 weeks after immunization (FIGS. 14A-14B). Even more striking, more than one year post immunization, mice immunized with amph-eOD retained significant populations of eOD-specific IgA plasma cells resident in the FRT and in the BM, whereas eOD-immunized mice showed negligible ASCs in either niche (FIG. 4E).

CDNs are in clinical trials as immunostimulators for cancer therapy but have yet to be used with vaccines in humans. Accordingly, a similar study was next carried out using an ISCOMs-like saponin adjuvant called SMNP [47], which has a nanoparticle structure and composition similar to the Matrix M adjuvant in advanced clinical testing for SARS-CoV-2 vaccines by Novavax [48] (FIG. 4F). Like CDNs, ISCOM-based adjuvants have been shown to be effective intranasal adjuvants in preclinical studies [49, 50]. Similar to the findings with cdGMP, i.n. immunization with amph-eOD and SMNP induced striking serum eOD-specific IgG and IgA titers of ˜10⁶ and ˜10⁴, respectively, greatly exceeding those induced by unmodified eOD at all timepoints pre- and post-boost (FIG. 4G). Amph-eOD/SMNP immunization also induced robust long-term mucosal IgG and IgA responses in the vaginal tract (FIG. 4H) and rectal mucosa (FIG. 4I), with amph-eOD post-boost titers consistently ˜10³-fold higher than those from eOD in the vaginal mucosa and 10-100-fold higher in fecal samples. After 35 weeks, the FRT and BM were analyzed by ASC ELISPOT, again showing significantly elevated numbers of eOD-specific IgA plasma cells in the FRT (P<0.05) and BM (P<0.1) of mice immunized with amph-eOD compared to eOD (FIG. 4J). Strikingly, with both cdGMP and SMNP adjuvants, the population of IgA plasma cells established in the female reproductive tract was similar or greater in magnitude to that in the bone marrow (FIGS. 4E-4J).

Taken together, these studies indicate that intranasal immunization with amph-conjugated antigen can promote robust long-term systemic and mucosal antigen-specific humoral immunity in mice with multiple adjuvants.

Recently, some concerns have arisen from clinical studies of the SARS-CoV-2 mRNA vaccines regarding the possibility of antibody responses against PEG included in vaccine formulations, which might induce allergic reactions in human volunteers. Thus, serum samples from the studies above using saponin or cdGMP adjuvants were analyzed for the presence of anti-PEG IgG. Despite the use of strong adjuvants, anti-PEG responses elicited by amph-eOD were barely above background (FIG. 14C).

### Example 6: Induction of High Levels of Neutralizing Antibodies Against SARS-CoV-2 in the Respiratory Mucosa by Amph-Vaccination

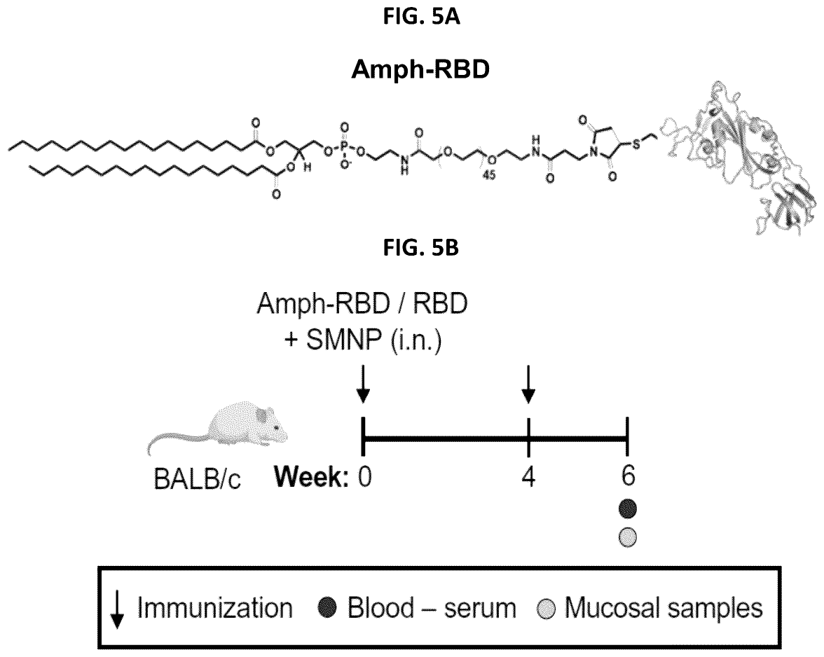

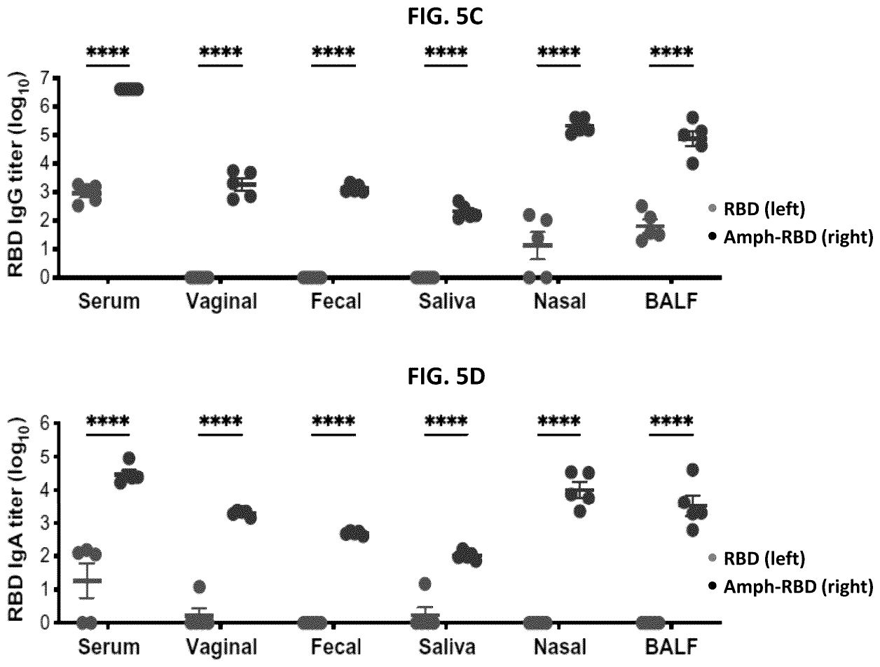

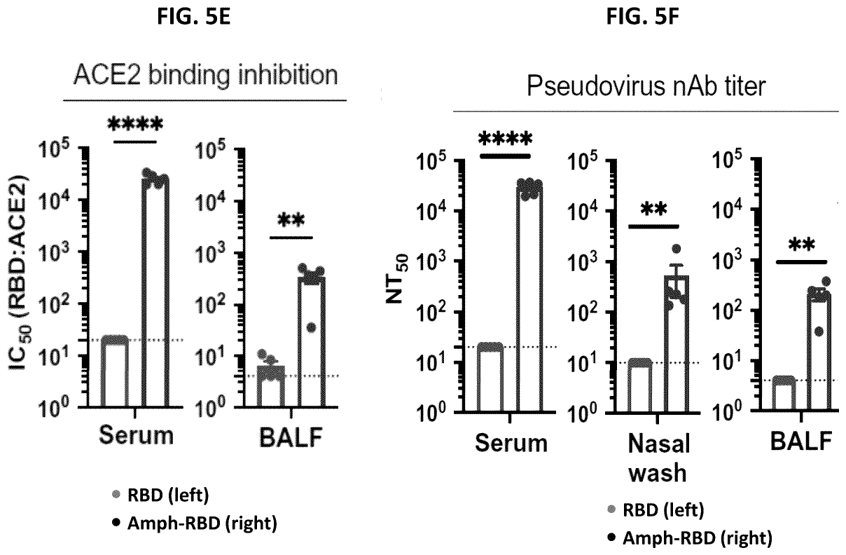

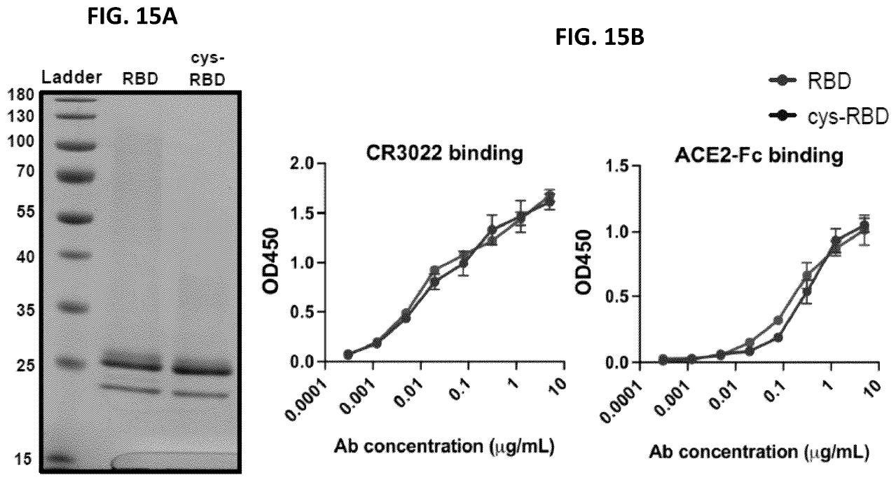

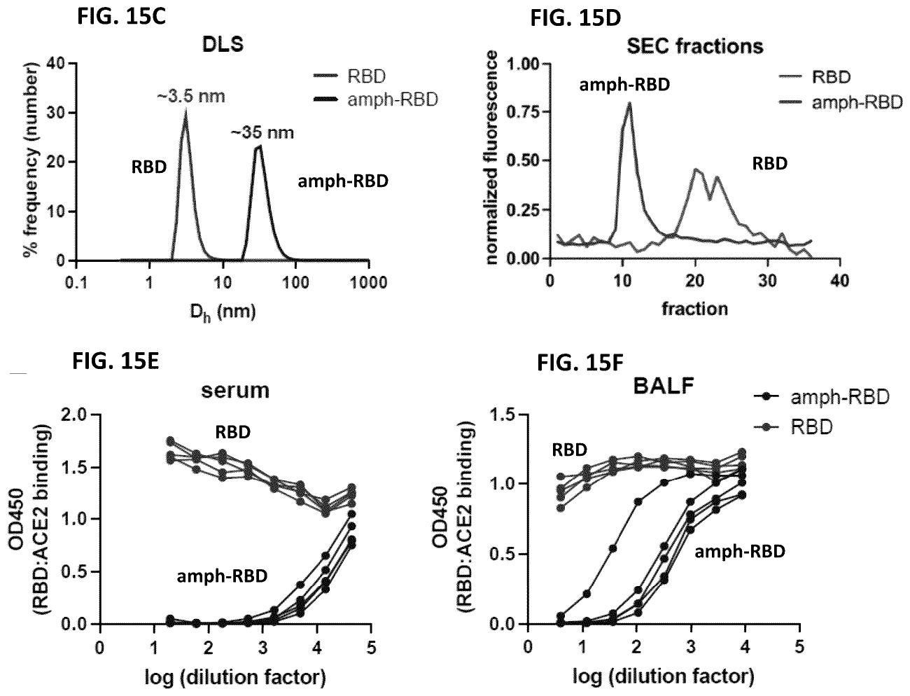

eOD is a germline targeting immunogen designed to initiate priming of human B cells with the capacity to produce broadly neutralizing antibodies similar to the CD4 binding site bnAb VRC01 [38-41], but this immunogen cannot induce neutralizing antibody responses in wild-type mice, due to genetic differences in the CDR3 regions of murine vs. human antibodies. Further, responses induced in the local respiratory mucosa by i.n. immunization are not relevant for protection from HIV. These considerations provided the motivation to test the utility of amph-conjugation in the setting of vaccines for SARS-CoV-2, as WT mice readily produce neutralizing antibodies against this virus and nAb responses in the nasal passages and airways are highly relevant for protection [51-53]. The receptor binding domain (RBD) of the SARS-CoV-2 spike protein was chosen as the target antigen to incorporate into the amphiphile platform as it is the target of most human neutralizing antibodies [54]. Soluble RBD protein is known to be poorly immunogenic [55, 56]; it was therefore tested whether amphiphile conjugation of RBD would enhance its immunogenicity and promote protective systemic and respiratory mucosal antibody responses in tandem. To this end, an engineered RBD immunogen recently developed was employed, which is expressed in Pichia pastoris and expresses at much higher levels and exhibits substantially greater stability than the wild-type RBD sequence [57]. Modifying the RBD immunogen with an N-terminal cysteine did not impact its production, stability, or antigenicity profile (FIGS. 15A-15B), and enabled conjugation of the protein with maleimide-functionalized PEG_{2K}-DSPE (FIG. 5A). Similar to amph-eOD, conjugated amph-RBD formed ˜35 nm diam. micelles in aqueous solution, facilitating purification from unreacted RBD (˜5 nm) by SEC (FIGS. 15C-15D).

To assess the immunogenicity of amph-RBD, BALB/c mice were immunized i.n. with amph-RBD or RBD combined with SMNP adjuvant at 0 and 4 weeks; at wk 6, serum and mucosal samples were collected and assayed for RBD-specific IgG/A titers and pseudovirus neutralization (FIG. 5B). As shown in FIGS. 5C-5D, amph-RBD dramatically outperformed soluble RBD for eliciting antigen-specific serum and mucosal IgG and IgA responses. Serum Ig levels were three orders of magnitude greater for amph-RBD vs. RBD, and importantly, amph-RBD elicited potent IgG and IgA responses in nasal washes and bronchiolar lavage fluid (BALF), where soluble RBD immunization elicited weak or no responses (FIGS. 5C-5D). An ACE2-RBD binding inhibition assay revealed an IC50 for blocking ACE2 binding by RBD of ˜25,000 in the serum and ˜300 in the BALF from amph-RBD-immunized mice (FIG. 5E, FIGS. 15E-15F). Finally, analysis of SARS-CoV-2 pseudovirus neutralization revealed serum nAbs at titers of ˜30,000, and mean nasal and BAL nAb titers of ˜500 and ˜200, respectively (FIG. 5F). In contrast, intranasal immunization with soluble RBD elicited no detectable neutralizing response in any compartment (FIG. 5F). Thus, intranasal amph-RBD vaccination dramatically enhances the induction of neutralizing antibody responses at mucosal portals of entry for the SARS-CoV-2 virus.

### Example 7: Amph-Conjugated Vaccines Exhibit Enhanced Immunogenicity in Non-Human Primates

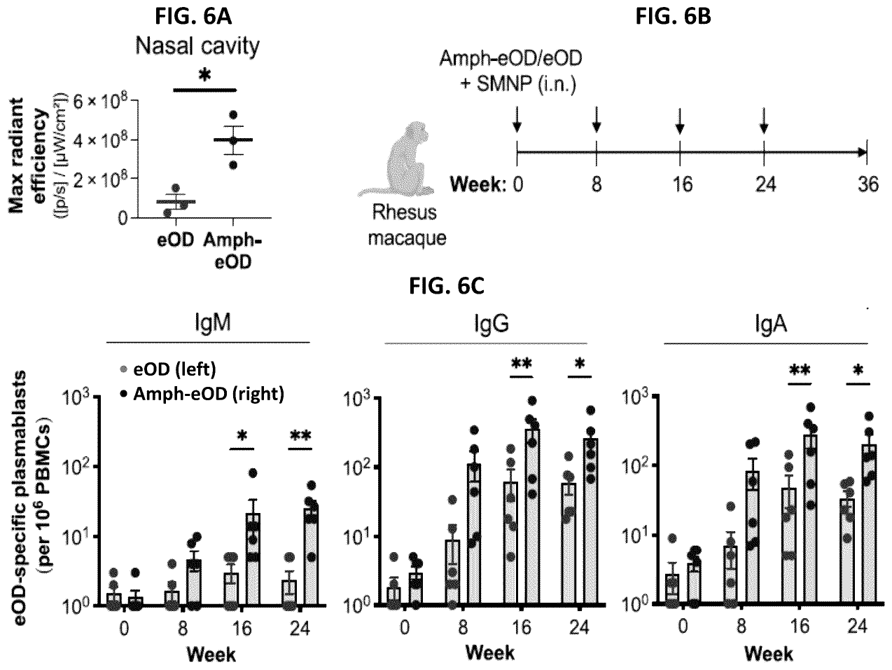

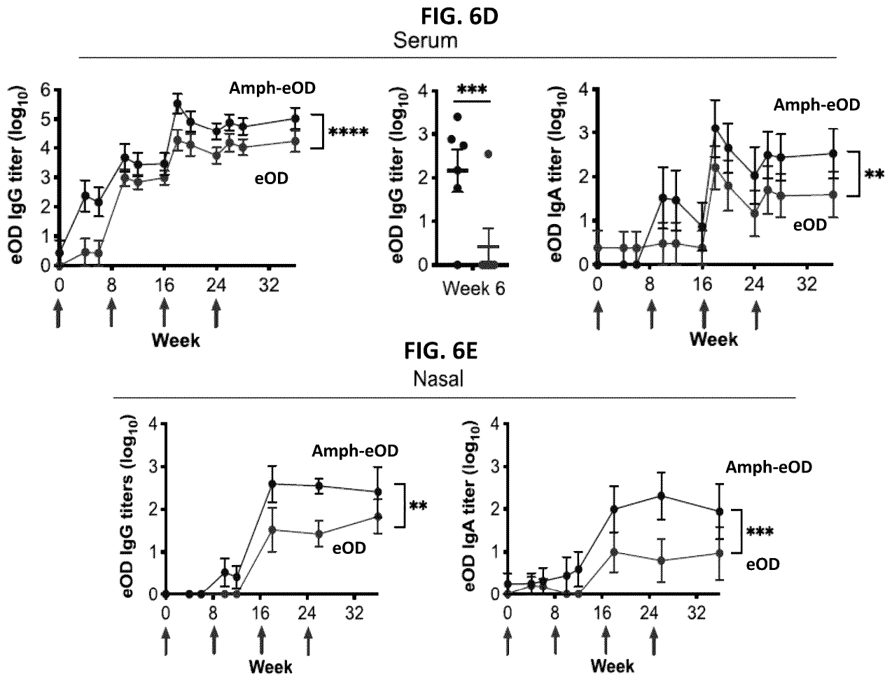

The systemic and mucosal antibody responses elicited by amph-conjugate vaccines in mice were compelling, but many vaccine technologies that are effective in small animals fail to translate well to larger animals and humans. Thus, whether amph-conjugates would also be effective in non-human primates (NHPs) was next evaluated, using the eOD immunogen. Trafficking of the amphiphile vaccine versus soluble protein after intranasal immunization was first evaluated in rhesus macaques. Alexafluor-labeled amph-eOD or soluble eOD was administered intranasally with SMNP adjuvant; after 24 h, the tonsils, adenoids, cervical LNs, axillary LNs, and nasal tissue including turbinates were collected and evaluated by IVIS imaging for fluorescence signal from the labeled immunogens. Similar to the observations in mice, amph-eOD was detected in the nasal tissue at a significantly higher level than eOD (FIG. 6A). Negligible signal was detected in the cervical LNs or axillary LNs (data not shown).

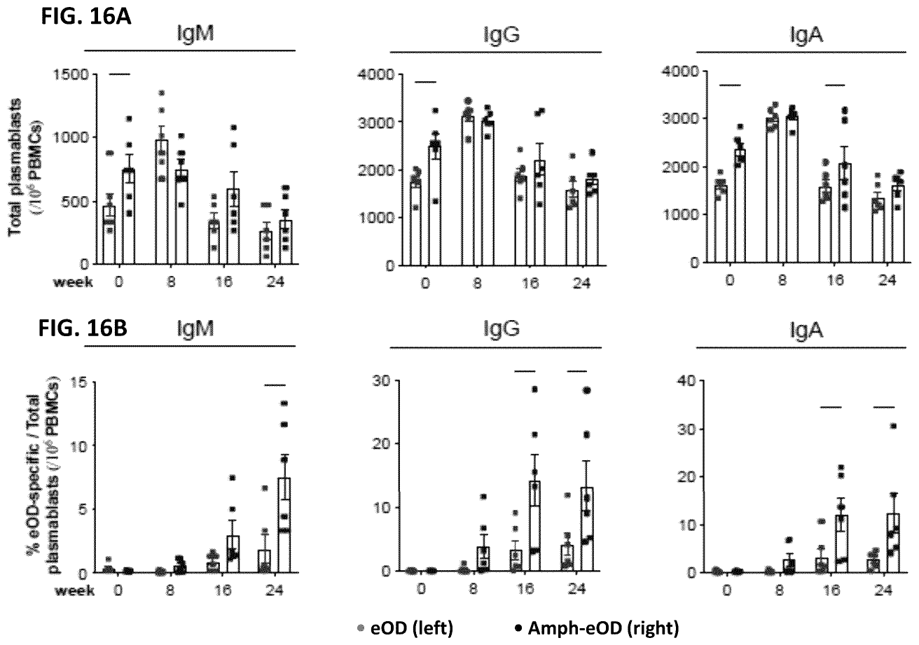

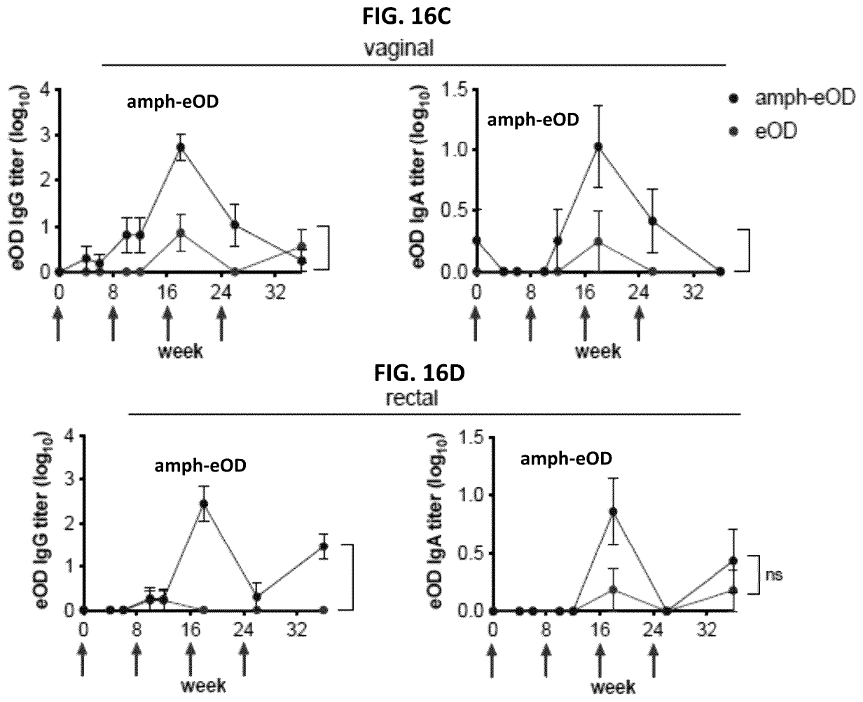

To assess vaccine immunogenicity, NHPs were immunized i.n. with amph-eOD or eOD combined with SMNP at 0, 8, 16, and 24 wks (FIG. 6B). PBMCs were collected 5 days after each immunization to assay plasma blast responses by antibody secreting cells (ASC) ELISPOT. Amph-eOD induced significantly higher eOD-specific IgM, IgG, and IgA plasma blast responses after the second and third boosts, quantified as total number of antigen-specific plasma blasts or as a percentage of total plasma blasts (FIG. 6C, FIGS. 16A-16B). In the serum, amph-eOD i.n. immunization seroconverted all animals following a single dose, whereas serum IgG titers primed by soluble eOD were near baseline until the first boost was administered (FIG. 6D). Antigen-specific serum IgG and IgA titers were consistently ˜10-fold higher in NHPs immunized with amph-eOD compared to eOD even following repeated boosting (FIG. 6D). In the nasal mucosa, IgG and IgA were ˜1 log higher in NHPs immunized with amph-eOD compared to eOD at wks 18 and 26 and were sustained after boosting (FIG. 6E). Distinct from the findings in mice, amph-eOD elicited sporadic vaginal and rectal IgG and IgA responses: while overall vaginal IgG, vaginal IgA, and rectal IgG from amph-eOD were significantly greater than eOD (p<0.01, p<0.05, and p<0.0001, respectively), these responses were not consistently sustained throughout the study (FIGS. 16C-16D). Altogether, these data in the closest available animal model to humans suggest that amph-conjugate intranasal immunization is a promising strategy for enhancing both systemic and mucosal immunity to subunit vaccines.

### Example 8: Synthesis of Amphiphilic Conjugate with HIV Trimer Antigen and Intranasal Immunization in Mice

The best immunogen candidates for eliciting broadly neutralizing antibodies against HIV are native-like trimers such as MD39 SOSIP. Thus, motivated by promising results with amph-eOD, an amphiphile conjugate with HIV MD39 SOSIP trimer, which is a much larger protein antigen, was synthesized. For conjugation of this larger trimer protein, a longer linker was employed to avoid steric hindrance upon incorporation into the amphiphile platform.

Amph-MD39 synthesis and purification: HIV MD39 SOSIP trimer with C-terminal cysteine (≥1 mg/ml) was first reduced with 10 molar equivalents of tris(2-carboxyethyl)phosphine (TCEP) for 15 minutes at 25° C. TCEP was removed through centrifugal filtration using 10 kDa molecular weight cutoff (MWCO) Amicon spin filters while washing the protein three times with phosphate-buffered saline (PBS). Protein (1 to 5 mg/ml) was then reacted with 5 molar equivalents of DBCO-PEG4-maleimide (dibenzocyclooctyne-PEG4-maleimide, MW 674.74 Da) (Sigma) in PBS for 18 hours at 4° C. Unreacted maleimide-PEG4-DBCO was then removed using 10 kDa MWCO Amicon spin filters, and the product was analyzed by UV-Vis spectrophotometry (Nanodrop One, Thermo Fisher Scientific) for the presence of a DBCO peak at 309 nm. MD39-DBCO was then mixed (≥1 mg/ml) with 5 molar equivalents of dried DSPE-PEG2K-azide (1,2-distearoyl-sn-glycero-3-phosphoethanolamine-N-[azido(polyethylene glycol)-2000], MW 2816.519 Da) (Avanti Polar Lipids) in PBS for 2 hours at 25° C. with intermittent vortexing, followed by gentle mixing for 18 hours at 4° C. The product was then measured again by UV-Vis: the absence of a DBCO peak at 309 nm verified the reaction had progressed to completion. MD39 concentration was determined by the protein peak at 280 nm and corrected for the background lipid absorbance from 310-500 nm. Protein amphiphile was purified by affinity chromatography using azide-functionalized agarose beads in a gravity column eluted with PBS in order to separate unreacted MD39-DBCO amph-MD39. The conjugated protein-amphiphile was quantified by UV-Vis.

Mouse immunizations and blood collection: Immunization studies were carried out using age-matched 8- to 10-week-old female BALB/cJ mice (strain 000651) purchased from the Jackson Laboratory.

BALB/c mice were immunized intranasally by administering vaccines in 20 μl of phosphate-buffered saline (PBS; 10 μl per nare with 30- to 60-s interval between nares) with the mouse anesthetized in the supine position. Animals were primed on day 0 and boosted on day 42 and 84 with a 5-μg dose of MD39 (soluble MD39 or amph-MD39) combined with 5 μg of saponin monophosphoryl lipid A (MPLA) nanoparticle (SMNP) adjuvant. For longitudinal immune monitoring, blood and mucosal samples were collected bi- or triweekly for ELISA. Blood was collected by cheek or retro-orbital bleed; serum was isolated using serum separator tubes and centrifuged at 10,000 g for 5 min to collect supernatant. Vaginal mucosal fluid was collected from anesthetized mice by vaginal lavage using 75 μl sterile PBS (3×25 μl instillations, each aspirated three to five times) combined with 5 μl of 25× protease inhibitor (EDTA-free SIGMAFAST Protease Inhibitor Cocktail Tablets, Sigma-Aldrich); fluid was centrifuged at 12,000×g for 10 minutes at 4° C. to collect supernatant.

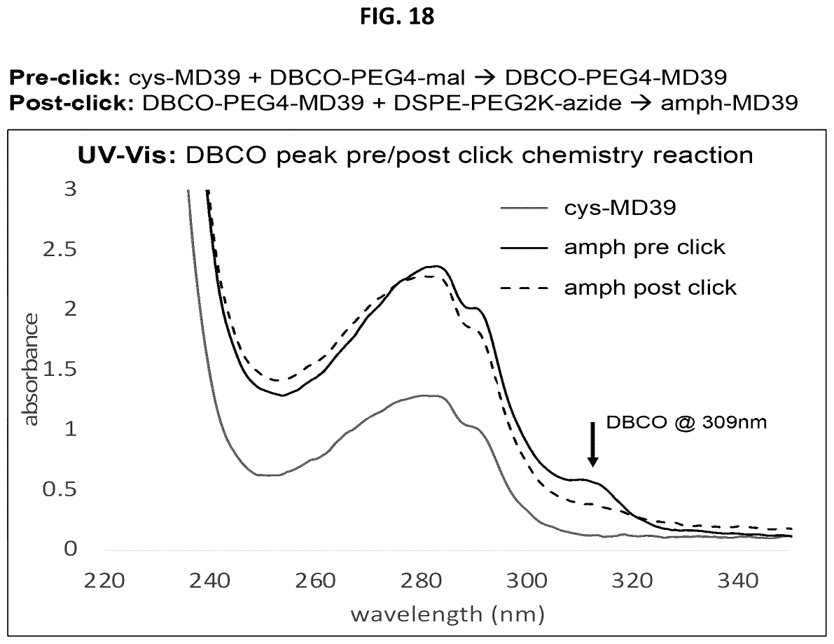

Results: Inclusion of a second linker was effective for efficient synthesis of amphiphile-MD39 trimer conjugates. MD39 with C-terminal cysteine was first reacted with DBCO-PEG4-maleimide linker to form intermediate product DBCO-PEG4-MD39, prior to click chemistry reaction with the functionalized lipid DSPE-PEG2K-azide to form final product amph-MD39 (FIGS. 17A-17B). The intermediate product DBCO-PEG4-MD39 was clearly identified by UV-Vis spectrophotometry by the co-presence of an MD39 peak at 280 nm and DBCO peak at 309 nm; the post click amph-MD39 product was identified by an MD39 peak at 280 nm and absence of DBCO peak at 309 nm, indicating the reaction went to completion (FIG. 18).

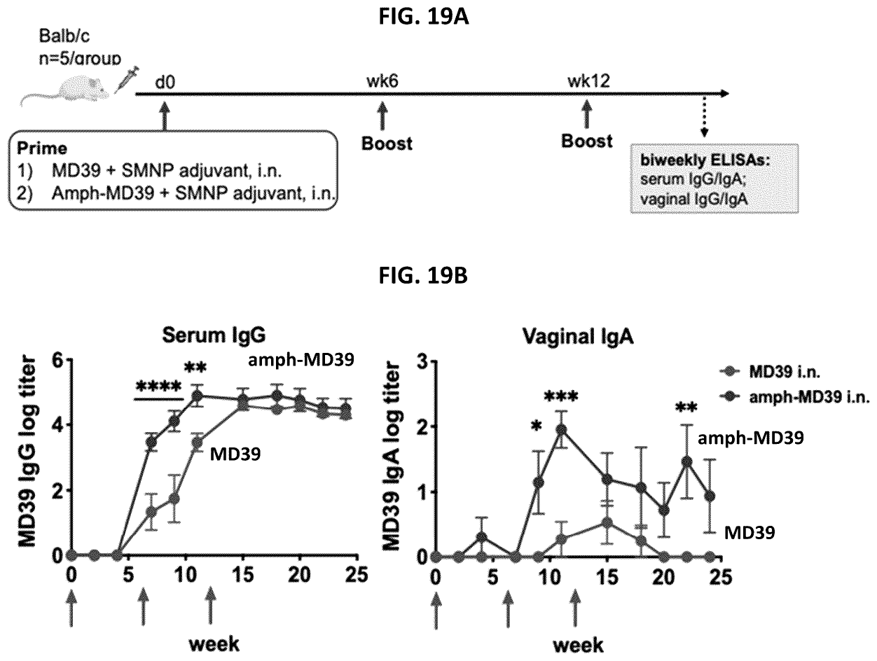

Intranasal immunization with amph-MD39 elicited significantly greater serum and mucosal antigen-specific antibody responses compared to soluble MD39 protein (FIG. 19B). Amph-MD39 elicited significantly greater serum IgG at weeks 7 (p<0.0001), 9 (p<0.0001), and 11 (p<0.01) post-prime and significantly greater vaginal mucosal IgA at weeks 9 (p<0.05), 11 (p<0.001), and 22 (p<0. 01) post-prime compared to soluble MD39. These results indicate that intranasal immunization with amphiphile conjugates of larger proteins such as MD39 trimers (roughly 10-fold greater MW than eOD or RBD monomers) can elicit robust antibody responses in both the serum and genitourinary mucosa.

By pairing a native trimer immunogen known to elicit broadly neutralizing antibodies with amphiphile conjugation for enhanced transmucosal uptake, this immunization strategy can be employed for eliciting broadly neutralizing antibodies against HIV in clinically-relevant sites of transmission such as the genitourinary mucosa.

### Example 9: Discussion

It was previously demonstrated that linking peptide antigens to amphiphilic lipid tails promotes albumin-mediated transport into lymphatics following parenteral injection, thereby enhancing antigen-specific T cell responses that are critical for cancer immunity [25, 27, 29]. Here, it was surprisingly found that this strategy can be employed with much larger protein immunogens relevant for humoral immunity, and that ‘albumin hitchhiking’ can be applied to greatly enhance intranasal delivery of immunogens by exploiting another natural transport mechanism of endogenous albumin—its capacity to be transcytosed across the mucosal epithelium by the neonatal Fc receptor (FcRn) [31, 42]. Amph-proteins showed prolonged residence in the nasal tissue following i.n. administration in both mice and non-human primates. In mice, this persistence was demonstrated to be linked to increased transport across the mucosal barrier and greater uptake in the NALT. The NALT is a secondary lymphoid organ located on the dorsal side of the soft palate underlying the nasal passage in rodents, analogous to the Waldeyer's Ring in primates and humans [16]. In mice the NALT consists of focal aggregates, whereas in primates the Waldeyer's Ring is more abundant consisting of tonsils and adenoids [15, 58]. Importantly, the NALT, tonsils, and adenoids all serve as key sites for initiation and orchestration of local mucosal antigen-specific immune responses [3, 59, 60]. Substantial increases in germinal center B cell and Tfh cell responses were found in the NALT following i.n. immunization with amph-conjugate immunogens when compared to free proteins. This increased antigen delivery and local immune priming correlated with greatly enhanced systemic IgG and IgA responses, as well as mucosal antibody responses, in both mice and non-human primates. Amph-modification of protein immunogens enabled intranasal immunizations to elicit strong serum IgG responses in conjunction with robust mucosal IgA responses. This is of great interest as many infectious diseases such SARS-CoV-2, influenza, rotavirus, and cholera are thought to require a combination of mucosal IgA and serum IgG antibodies for optimal protection [1-7]. Thus, the ability to activate both systemic IgG and mucosal IgA is likely to be of value in diverse vaccines.

Amph-proteins overcome a major obstacle to mucosal vaccine development: delivery of antigens across the mucus and epithelial barrier to the underlying mucosal immune compartment [18, 19]. In addition to efficient mucociliary clearance mechanisms, mucosal surfaces are lined with epithelial monolayers formed by intercellular tight junctions that prevent macromolecular uptake by diffusion [61]. Thus, transport of molecules across the nasal mucosal epithelium is thought to be restricted to active transport of small soluble proteins by goblet cells [22, 62], and transport of larger inert particulates by differentiated microfold cells (M cells). Similar to Peyer's Patches in the gut, M cells are also found lining the nasal cavity, both in the turbinate epithelium and in follicle-associated epithelium overlaying the NALT where they sit atop subepithelial domes (SED) of organized mucosal lymphoid tissue and act as ‘antigen delivery cells’ [16, 63, 64]. Here, M cells acquire antigen from the nasal mucosal lumen, transcytose it across the submucosal epithelium, and then hand off antigen to underlying DCs, macrophages, B cells, and other APCs in the SED. Following intranasal administration, a significant amount of amph-eOD was observed to be concentrated in the nasal turbinates, which may have allowed for M cell capture and transcytosis to serve as another mechanism for intranasal amph-eOD uptake [62, 65]. However, FcRn expressing columnar epithelial cells are much more abundant than M cells in the respiratory mucosa [62]. This, in combination with the data showing a clear dependence of amph-eOD uptake and immune responses on FcRn, indicates FcRn-mediated transcytosis is a more efficient pathway for antigen delivery in the nasal mucosa. Albumin-bound amph-antigens transcytosed by respiratory epithelial cells would be released at the basolateral surface, where they can then be taken up by underlying APCs. Interestingly, APCs such as macrophages, DCs, and B cells—where the highest amph-eOD uptake was observed—also express high levels of FcRn [66].

Recognized for its role in recycling and extending the half-lives of IgG and albumin, FcRn is increasingly targeted as a means to alter drug delivery and drug pharmacokinetics [30, 31, 67]. To date, the focus has largely been on developing engineered therapeutic monoclonal antibodies (i.e., Fc-fusions) with altered FcRn binding affinities or drug-albumin fusions, which extend serum half-life by exploiting FcRn-mediated recycling in the blood and increasing overall molecular weight to reduce the rate of kidney clearance. More recently, the FcRn transcytosis pathway has been explored for non-invasive protein delivery via FcRn-mediated transcytosis [43, 68-70]. For example, Pridgen et al. observed ˜10-fold higher uptake across the intestinal epithelium with FcRn-targeted nanoparticles versus non-targeted nanoparticles as a means to orally deliver encapsulated insulin across the intestinal mucosa in mice [71], while Bern et al. found that an engineered albumin-protein fusion with improved FcRn binding exhibited enhanced uptake across the nasal epithelium and increased serum half-life in mice [43].

More directly relevant to the present study, Roopenian and Zhu demonstrated that fusions of protein antigens with antibody Fc domains can enhance intranasal vaccination against HSV-2 [72] and HIV gag [73]. These antigen-Fc fusions enhanced systemic antibody and T cell responses to i.n. immunization, and mucosal antibody in BALF and vaginal fluid, but to our knowledge this approach has not been evaluated for efficacy in large animal models. An important distinction between approaches solely leveraging FcRn interactions and the amph-vaccine approach studied here is that Fc or albumin fusions administered to airway surfaces are delivered not only to the local mucosal lymphoid tissues but also reach the systemic circulation, and thereafter exhibit circulation times in the blood seen for antibodies/albumin; this has motivated the use of Fc and albumin fusions for delivery of systemic therapeutics such as erythropoietin [69,70]. Such broad distribution is problematic for vaccines, however: vaccine adjuvants by design provide very localized inflammatory cues to avoid systemic toxicity, but if antigens co-administered with these adjuvants do not also remain localized, a competing tolerogenic response can develop in uninflamed distal lymphoid tissues such as lymph nodes and spleen [74]. By contrast, the lipid tail of amphiphile conjugates promotes cell membrane interactions that prevent systemic dissemination of these conjugates. Here, localized stimulation of immune responses was observed following i.n. administration of amph-proteins, which activated responses in the NALT but did not significantly reach even the nearby draining cervical LNs or accumulate in tissues such as the spleen, liver, and intestines, indicating negligible systemic distribution.

Development of an amph-RBD COVID vaccine demonstrated the ability of this amph protein vaccine platform to induce functional neutralizing antibody responses at mucosal sites of respiratory pathogen entry. Clinical studies have shown that mucosal IgA is a strong correlate of protection against SARS-CoV-2 [6, 13, 14], but to date, most COVID vaccines have not focused on targeting mucosal tissues and few have been shown to induce functional neutralization at mucosal sites [53, 75, 76]. Amph-RBD immunization induced striking IgG and IgA antibody responses, including nAbs, in both serum and the upper and lower respiratory mucosa in mice. Thus, intranasal amph-RBD vaccination is a promising approach for eliciting mucosal protection against COVID. Additionally, needle-free mucosal vaccination provides practical advantages over parenteral vaccination in cases where mass vaccination is needed, such as the current global COVID-19 pandemic: easier administration, delivery that does not require personnel with medical training, better compliance, and avoiding risks of spreading blood-borne infections through needle contamination, all leading to better vaccination rates [77].

A limitation of these studies is the inherent challenge of immunological differences between animal models and humans. In mice, amph-protein immunization elicited not only robust local mucosal Ig responses, but also stimulated long-lived, high titer IgG and IgA at distal vaginal and rectal mucosal sites, accompanied by generation of resident antibody-secreting cells. By contrast, i.n. amph-protein immunization in non-human primates elicited enhanced systemic and nasal IgG and IgA responses compared to soluble protein administration, but distal mucosal responses in the vaginal tract and rectum were not sustained. However, such “common mucosal immunity” has been reported in small studies in macaques [1, 78-81] and humans. For example, i.n. immunization with the strong mucosal adjuvant cholera toxin B (CTB) led to volunteers showing antibody responses in urine or vaginal secretions [82, 83]. CTB has not advanced as an intranasal adjuvant due to its associated risk of triggering Bell's palsy [82], but these data suggest that with appropriate adjuvants, distal mucosal responses can be elicited in humans. Despite this limitation, the strong systemic and local mucosal antibody priming observed here in NHPs following intranasal amph-protein administration combined with the saponin adjuvant SMNP, an adjuvant currently in GMP development for a first-in-humans clinical trial, indicate that this approach is valuable for human vaccines.

Altogether, these results demonstrate that employing amphiphile-protein vaccines to deliver antigen across the mucosal epithelium presents an excellent strategy to promote mucosal immunity against HIV, SARS-CoV-2, and other infectious diseases.

### Example 10: Materials and Methods

Study Design. The major objective of this study was to evaluate the effect of modifying protein antigens with an amphiphilic PEG-lipid tail on systemic and mucosal immune responses elicited by intranasal vaccination in small and large animal models, and to define mechanisms of action underlying the action of these modified immunogens. Mice and non-human primates were immunized with clinically-relevant subunit protein immunogens combined with saponin or alternate adjuvants, and early local responses (antigen uptake, T cell priming, and germinal center induction) and later events (serum and mucosal antibody, plasma blast, plasma cell) responses were assessed over time. For mechanistic studies, we utilized fluorescently labeled proteins enabling immunogen trafficking in tissues and genetic knockout mouse models to dissect key pathways in the immune response.

### Immunogen Synthesis and Characterization

HIV eOD. eOD-GT8 gp120 protein was synthesized as previously described [84, 85]. The eOD protein, with a free N-terminal cysteine and C-terminal PADRE universal helper T cell epitope (AKFVAAWTLKAAA), was expressed in HEK cells and purified on a Nickel affinity column followed by size-exclusion chromatography on a Superdex 75 10/300 column (GE Healthcare).

eOD gp120 monomer (with PADRE epitope italicized
and underlined): MW 21.787 kDa
(SEQ ID NO: 1)
ETGCHHHHHHGGDTITLPCRPAPPPHCSSNITGLILTRQGGYSNDNTVI
FRPSGGDWRDIARCQIAGTVVSTQLFLNGSLAEEEVVIRSEDWRDNAKS
ICVOLNTSVEINCTGAGHCNISRAKWNNTLKQIASKLREQYGNKTIIFK
PSSGGDPEFVNHSFNCGGEFFYCDSTQLFNSTWENSTGSAKFVAAWTLK
AAA

SARS-CoV-2 RBD. An engineered RBD protein (‘RBD-L452K-F490W’) was produced in Komagataella phaffii (Pichia pastoris). This strain was cultivated in 200 mL flask culture and

secreted protein was purified as previously described [57]. For amphiphile conjugation, the RBD was genetically modified to include an N-terminal cysteine residue.

SARS-CoV-2 RBD monomer: MW 22.684 kDa
(SEQ ID NO: 2)
CITNLCPFGEVENATRFASVYAWNRKRISNCVADYSVLYNSASFSTFKC
YGVSPTKLNDLCFTNVYADSFVIRGDEVRQIAPGQTGKIADYNYKLPDD
FTGCVIAWNSNNLDSKVGGNYNYKYRLFRKSNLKPFERDISTEIYQAGS
TPCNGVEGENCYWPLQSYGFQPTNGVGYQPYRVVVLSFELLHAPATVCG
PKKSTN

HIV MD39 SOSIP: MD39 SOSIP is a HIV native-like trimer antigen (J. M. Steichen et al., Science. 366 (2019), the entire contents of which are incorporated herein by reference). MD39 SOSIP trimer has molecular weight (MW) of about 217.018 kDa.

MD39's monomer sequence: (monomer MW 72.339 kDa)
(SEQ ID NO: 3)
AENLWVTVYYGVPVWKDAETTLFCASDAKAYETEKHNVWATHACVPTDP
NPQEIHLENVTEEFNMWKNNMVEQMHEDIISLWDQSLKPCVKLTPLCVT
LQCTNVINNITDDMRGELKNCSFNMTTELRDKKQKVYSLFYRLDVVQIN
ENQGNRSNNSNKEYRLINCNTSAITQACPKVSFEPIPIHYCAPAGFAIL
KCKDKKFNGTGPCPSVSTVQCTHGIKPVVSTQLLLNGSLAEEEVIIRSE
NITNNAKNILVQLNTPVQINCTRPNNNTVKSIRIGPGQAFYYTGDIIGD
IRQAHCNVSKATWNETLGKVVKQLRKHFGNNTIIRFAQSSGGDLEVTTH
SFNCGGEFFYCNTSGLFNSTWISNTSVQGSNSTGSNDSITLPCRIKQII
NMWQRIGQAMYAPPIQGVIRCVSNITGLILTRDGGSTNSTTETFRPGGG
DMRDNWRSELYKYKVVKIEPLGVAPTRCKRRVVGRRRRRRAVGIGAVSL
GFLGAAGSTMGAASMTLTVQARNLLSGIVQQQSNLLRAPEPQQHLLKDT
HWGIKQLQARVLAVEHYLRDQQLLGIWGCSGKLICCTNVPWNSSWSNRN
LSEIWDNMTWLQWDKEISNYTQIIYGLLEESQNQQEKNEQDLLALDGTK
HHHHHHC

Amphiphile conjugation and labeling. eOD and RBD protein antigens with N-terminal cysteines (≥1 mg/ml) were first reduced with 10 molar equivalents of tris(2-carboxyethyl)phosphine (TCEP) for 15 minutes at 25° C. TCEP was removed through centrifugal filtration using 10 kDa MWCO Amicon spin filters while washing the protein 3× with PBS. Proteins (1-5 mg/ml) were then reacted with 4 equivalents of dried DSPE-PEG2K-maleimide (1,2-distearoyl-sn-glycero-3-phosphoethanolamine-N-[maleimide(polyethylene glycol)-2000]) (Avanti Polar Lipids) in PBS for 2 h at 25° C. with intermittent vortexing, followed by gentle mixing for 18 hr at 4° C. Protein amphiphiles were purified by size exclusion chromatography (SEC) using a Sepharose CL6B (Sigma-Aldrich) gravity column eluted with PBS. The conjugated protein amphiphile micelle and unconjugated protein peaks were detected by tryptophan fluorescence (exc: 280 nm/em: 340 nm). Micelle peak fractions were pooled, concentrated through centrifugal filtration using 10 kDa MWCO Amicon spin filters, and quantified by UV-Vis spectrophotometry (Nanodrop One, Thermo Scientific). Particle size was characterized by dynamic light scattering (Zetasizer Nano, Malvern).

Labeled eOD proteins and protein amphiphiles were prepared using AF647 NHS ester (ThermoFisher Scientific) by reaction of fluorophore with eOD or amph-eOD (≥1 mg/ml) in 0.1M sodium bicarbonate buffer for 1 h at 25° C., per the manufacturer instructions. VRC01 was synthesized as previously described [86]; labeled VRC01 was prepared using Pierce NHSRhodamine (ThermoFisher Scientific) by reaction of the fluorophore with human VRC01 (≥1 mg/ml) in PBS for 1 h at 25° C., per the manufacturer instructions. Labeled proteins were purified by centrifugal filtration using 10 kDa Amicon spin filters; degree of labeling (DOL) was characterized by UV-Vis spectrophotometry and confirmed to be ≥1.0.

Adjuvants. The STING agonist adjuvant bis-(3′-5′)-cyclic dimeric guanosine monophosphate (cdGMP) was purchased from InvivoGen. Saponin MPLA nanoparticle adjuvant (SMNP) was synthesized as previously described [87].

Albumin binding: affinity chromatography. Albumin binding of conjugates was evaluated using albumin-immobilized agarose affinity chromatography as previously described [25]. Pierce NETS-activated agarose resin (ThermoFisher Scientific) was functionalized with albumin by adding 26.4 mg BSA in 4.4 ml PBS directly to 330 mg agarose, per the manufacturer instructions. The resin reaction was mixed for 1 h at 25° C. followed by 4° C. overnight, then quenched with 1M Tris-HCl (pH 8.0) followed by extensive washing with PBS. Next, AF647-labeled eOD or amph-eOD was applied to the albumin-functionalized resin (0.3 μM final concentration in 2 ml column volume) and incubated with end-over-end mixing for 2 h at 37° C. Eluent was collected following column centrifugation at 1000×g for 2 min. The amount of protein or amphiphile conjugate retained in the column was determined by measuring AF647 fluorescence (640/670 nm) of the eluent vs starting sample on a fluorescent plate reader and normalizing by DOL.

Membrane insertion in splenocytes. Amphiphile insertion into cell membranes was evaluated in vitro in murine splenocytes isolated from naive mice. Single cell suspensions were incubated at 5×10⁶ cells/ml (1×10⁶ cells/well in a 96-well plate) in cRPMI (RPMI-1640+10% FBS+1% penicillin/streptomycin) containing 25, 100, or 250 nM AF647-eOD or AF647-amph-eOD for 1 h at 37° C. Cells were washed 1× with PBS, stained with Live/Dead Aqua (Invitrogen) at 1:1000 in 100 μl PBS for 15 min at 25° C., washed 1× in FACS buffer (PBS+1% BSA), then stained with Rhodamine-VRC01 at 1.0 μg/106 cells in 100 μl FACS buffer for at 4° C. Cells were then washed 2×, fixed with 2% paraformaldehyde, and stored at 4° C. until flow cytometry analysis on a BD LSR Fortessa.

Albumin-Neonatal Fc receptor (FcRn) binding measurements. To measure FcRn binding, 96-well enzyme-linked immunosorbent assay (ELISA) plates (Corning, #3690) were coated with 5.0 μg/ml streptavidin in phosphate-buffered saline (PBS) and incubated for 4 hours at 25° C., blocked for 18 hours at 4° C. with 1% casein in PBS (G-biosciences, 786-194), then washed three times with PBS+0.05% Tween 20 (pH 5.5). Biotinylated human FcRn (ACRO Biosystems, FCMH82W4) was added at 5 μg/ml in 1% casein in PBS (pH 5.5) and incubated for 2 hours at 25° C. prior to washing. Human albumin (Sigma, A3782, serially diluted 5-0 μg/ml) was pre-incubated for 2 hours at 25° C. with fluorescein isothiocyanate (FITC)-labeled 1,2-distearoylsn-glycero-3-phosphoethanolamine-N-[poly(ethylene glycol)-2000] (DSPE-PEG2K-FITC, Creative PEGworks, PLS-9927, serially diluted 10-0 μM) in 1% casein in PBS (pH 5.5), then added to the FcRn-coated plates and incubated for an additional 2 hours at 25° C. Goat anti-Human Albumin Antibody, horseradish peroxidase (HRP)-Conjugated (Bethyl Laboratories, A80-129P), diluted 1:3000 in 1% casein in PBS, was added and incubated for 30 minutes at 25° C. Plates were washed three times before adding tetramethylbenzidine (TMB) substrate (Thermo Fisher Scientific, 34028) followed by 2 N H₂SO₄ as a stop solution. Absorbance was measured at 450 nm.

### Murine Studies

Animal strains. All procedures were approved by the Massachusetts Institute of Technology Institutional Animal Care and Use Committee (IACUC) following local, state, and federal regulations. Immunization studies were carried out using age-matched 8-10 wk old female BALB/cJ mice (strain 000651), C57BL/6J mice (strain 000664), or FcRn−/− mice on a C57BL/6J background (strain 003982) purchased from The Jackson Laboratory.

IVIS trafficking. In vivo trafficking of AF647-labeled amph-eO D and eOD was evaluated following intranasal administration using an IVIS fluorescence imaging system (Perkin Elmer). Mice were fed an alfalfa-free diet (AIN-93M, Bio-Serv) for the duration of the study, starting 3 days before immunization, to eliminate background auto-fluorescence in the gut. BALB/c mice were immunized intranasally with 5 μg AF647-amph-eOD or AF647-eOD combined with 5 μg SMNP and compared to a naive control. Intranasal immunizations were administered dropwise in 20 μl PBS (10 μl per nare with 30-60 s interval between nares) with the mouse anesthetized in the supine position. Post-administration, mice remained anesthetized in the supine position for a minimum of 5 minutes to allow for uptake and prevent drainage. After 24 h, 48 h, 72 h, 7 d, and 11 d post-immunization, the following tissues were excised and AF647 fluorescence (radiant efficiency) was measured by IVIS: nasal cavity (snout minus lower mandible), cervical lymph nodes, intestines, mesenteric lymph nodes, liver, and spleen. The nasal cavity was imaged by removing the head from the mouse body, then removing and discarding the lower mandible from the snout; images were collected of the underside ventral surface of the upper palate (FIG. 2A, (i)).

To evaluate FcRn-dependence of amphiphile trafficking in the nasal mucosa, FcRn−/− mice were immunized intranasally with 5 μg AF647-amph-eOD combined with 5 μg SMNP and compared to WT mice (C57BL/6J) immunized intranasally with 5 μg AF647-amph-eOD or AF647-eOD combined with 5 μg SMNP. After 6 h, 24 h, and 72 h post-immunization, the nasal cavity was isolated as described above and AF647 fluorescence (radiant efficiency) was measured by IVIS.

Histology and fluorescence microscopy of nasal epithelium. Nasal cavity samples from FcRn−/− and C57BL/6 mice were processed for histology by FFPE (formalin fixed paraffin embedding) as follows: Samples were fixed in 10% neutral buffered formalin (NBF) for 24 h at 25° C., then transferred into 70% ethanol for storage at 4° C. Fixed samples were decalcified in 10% EDTA disodium salt dihydrate (Sigma) at pH 7.4 for 10 days at 4° C., changing the EDTA solution every 3 days. Decalcified tissues were embedded in paraffin and sliced into ˜5 μm coronal cross-sections using a microtome, starting 1 mm in from the nares and proceeding at 500 μm step intervals throughout the nasal cavity to a depth of 7.5 mm. Sections located 1.5-3 mm in from the nares were identified as the main site of vaccine deposition for detailed imaging (FIG. 2A, (ii)). Slices were mounted on a glass slide and stained with DAPI using Vectashield HardSet Antifade Mounting Medium with DAPI (Vector Laboratories), then imaged using a Leica SP8 laser scanning confocal microscope with 25× water objective or 63× oil objective. Images were processed in ImageJ.

ELISA for albumin quantification. To assay albumin concentrations in the nasal mucosa, nasal wash was collected from C57BL/6 or FcRn−/− mice as described above. Concentration of albumin in the nasal secretions was measured using a commercial mouse albumin ELISA kit (Abcam, cat #ab207620) per the manufacturer's instructions.

Flow cytometry analysis of NALT uptake. BALB/c mice were immunized intranasally with 10 μg AF647-eOD or AF647-amph-eOD combined with 5 μg SMNP. One and four days later, mice were euthanized and the NALT was isolated by excising the upper palate [88] and processing to a single cell suspension as follows: The upper palate was enzymatically and mechanically digested in 1 ml RPMI-1640 containing 0.8 mg/ml collagenase/dispase (Roche) and 0.1 mg/ml DNase (Roche) by first cutting into <1 mm chunks using fine-tipped spring-loaded scissors and then mashing in a 1.5 ml biomasher tube (Kimble). After incubating for 15 min at 37° C. with shaking, supernatant was removed and added to 10 ml FACS buffer (PBS+1% BSA) at 4° C.; the remaining tissue was subjected to a second round of digestion in 1 ml fresh enzyme mix for an additional 15 min at 37° C., then supernatant was removed and again added to cold FACS buffer. This FACS buffer solution was centrifuged at 500×g for 5 minutes to pellet cells, washed once in FACS buffer, passed through a 70 μm filter, and finally centrifuged and resuspended in FACS buffer in a Vbottom plate for antibody staining.

Cells were washed with PBS and first stained with Live/Dead Near-IR (Invitrogen) at 1:500 in 100 μl PBS for 15 min at 25° C., then treated with anti-mouse CD16/32 Fc block (TruStain FcX, BioLegend) at 1:100 in 50 μl FACS buffer for 10 min at 4° C. To identify different cell populations with vaccine uptake, cells were stained with the following antibodies at a dilution of 1:100 in 50 μl FACS buffer for 30 min at 4° C.: anti-mouse CD3ε APC-Cy7 (clone 145-2C11; BioLegend), B220 PerCP-Cy5.5 (RA3-6B2; BioLegend), CD45 BUV737 (30-F11; BD Biosciences), MHCII BV605 (M5/114.15.2; BioLegend), CD11b BV421 (M1/70; BioLegend), CD11c BV510 (N418; BioLegend), F4/80 BV711 (BM8; BioLegend), CD103 PE (2E7; BioLegend), CD8α BV786 (53-6.7; BD Biosciences), and CD169 PE-Cy7 (3D6.112; BioLegend). Cells were fixed with 2% paraformaldehyde and stored at 4° C. until flow cytometry analysis. Counting beads (Invitrogen) were added prior to running on a BD LSR Fortessa.

Flow cytometry analysis of NALT GC B cell and Tfh cell responses. FcRn−/− and C57BL/6 mice were immunized intranasally with 5 μg eOD or amph-eOD combined with 5 μg SMNP. After 12 days, mice were euthanized and the NALT was isolated and processed as described above. Cells were washed with PBS and first stained with Live/Dead Aqua (Invitrogen) at 1:500 in 100 μl PBS for 15 min at 25° C., then treated with anti-mouse CD16/32 Fc block (TruStain FcX, BioLegend) at 1:100 in 50 μl FACS buffer for 10 min at 4° C. To identify eOD-specific GC B cells, half the cells from each NALT sample were stained with the following panel in 50 μl FACS buffer for 30 min at 4° C.: anti-mouse CD3ε BV711 at 1:200 (clone 145-2C11; BioLegend), B220 PE-Cy7 at 1:200 (RA3-6B2; BioLegend), CD38 FITC at 1:200 (90; BioLegend), GL7 PerCP-Cy5.5 at 1:150 (GL7; BioLegend), eOD-tetramer PE at 1:100, and eOD-tetramer BV421 at 1:50. Fluorophore-labeled eOD tetramers were prepared by first reacting eOD with maleimide-PEG2-biotin (ThermoFisher) per the manufacturer's instructions, and then complexing 5 molar equivalents of biotinylated-eOD with 1 eq. of streptavidin-PE or streptavidin-BV421 (BioLegend) for 30 min at 25° C. To identify Tfh cells, half the cells from each NALT sample were stained with the following antibodies in 50 μl FACS buffer for 30 min at 4° C.: anti-mouse B220 BV510 at 1:200 (clone RA3-6B2; BioLegend), CD4 BV711 at 1:200 (GK1.5; BioLegend), CD44 PE-Cy7 at 1:200 (IM7; BioLegend), ICOS PE at 1:100 (7E.17G9; BioLegend), PD-1 BV650 at 1:50 (J43; BD Biosciences), and CXCR5-biotin at 1:50 (2G8; BD Biosciences) followed by streptavidin-BV421 at 1:100 (BioLegend).

Mouse immunizations and sample collection. BALB/c mice were immunized intranasally as described above. Mice were primed on day 0 and boosted on day 28 or 42 with a 5 μg dose of eOD or RBD combined with 25 μg cdGMP or 5 μg SMNP adjuvant, as indicated.

For longitudinal immune monitoring, blood and mucosal samples were collected bi- or triweekly for ELISA or PVNT antibody analysis, as indicated. Blood was collected by cheek or retroorbital bleed; serum was isolated using serum separator tubes and centrifuged at 10,000×g for 5 min to collect supernatant. Vaginal mucosal fluid was collected from anesthetized mice by vaginal lavage using 75 μl sterile PBS (3×25 μl instillations, each aspirated 3-5×) combined with 5 μl of 25× protease inhibitor (EDTA-free SIGMAFAST Protease Inhibitor Cocktail Tablets, Sigma); fluid was centrifuged at 12,000×g for 10 min at 4° C. to collect supernatant. Fecal wash was collected from mouse fecal pellets (4 pellets of ˜0.75 cm each per mouse) combined with 300 μl 1× protease inhibitor; samples were vortexed, incubated for 1 h at 4° C., vortexed a second time, then centrifuged at 13,000×g for 15 min at 4° C. to collect supernatant. Saliva wash was collected by dispensing 30 μl sterile PBS between the mouse's cheek and gumline (aspirated 3-5×), repeated on both sides, and combined with 10 μl of 2× protease inhibitor. All fluid samples were stored in aliquots at −80° C. for future analysis.

Post-euthanasia, bone marrow (BM) and female reproductive tract (FRT) tissue were collected to evaluate immune memory and resident plasma cell responses in the vaginal mucosa.

FRT was isolated from the vaginal opening to the ovaries, cut into 1-3 mm chunks using fine tipped spring-loaded scissors, and digested in 2 ml/sample of RPMI-1640 containing 2 mg/ml collagenase D (Roche), 0.6 U/ml Dispase II (StemCell Technologies), and 0.2 mg/ml DNase I (Roche) for 30 min at 37° C. with shaking. Samples were then centrifuged at 500×g for 5 minutes to pellet tissue and cells, supernatant discarded, and resuspended in 2 ml fresh digestion media for an additional incubation for 30 min at 37° C. with shaking. The digestion was quenched by adding an equal volume of RPMI-1640 containing 10% FBS and 1% penicillin/streptomycin. This solution plus remaining tissue was passed through a 70 μm cell strainer using the plunger end of a 1-ml syringe for additional mechanical digestion, then centrifuged at 500×g for 5 min and resuspended in 5 ml ACK lysis buffer for 5 min at 4° C. to lyse residual RBC. An equal volume of cRPMI was added to quench the ACK; samples were then centrifuged at 500×g for 5 min and rinsed 1× with cRPMI, passed through a 70 μm filter a second time, and finally centrifuged and resuspended a final time in cRPMI for counting and further analysis (ELISPOT, flow cytometry).

For RBD studies, nasal wash and bronchoalveolar lavage fluid (BALF) were collected to evaluate resident mucosal antibody responses in the upper and lower respiratory tract. Nasal wash was collected from 2×15 μl instillations of PBS, one in each nare (aspirated 3-5×), combined with 10 μl of 2× protease inhibitor. BALF was collected from 2×1 ml instillations of sterile PBS in the lungs using a 24G×.” catheter through the trachea. Both fluid samples were centrifuged at 12,000×g for 10 min at 4° C. to collect supernatant, then stored at −80° C.

ELISA analysis of mouse antibody titers. Anti-eOD and anti-RBD IgG and IgA binding titers were measured in mouse serum and mucosal samples (vaginal wash, fecal wash, saliva, nasal wash, and BALF) by ELISA. To capture eOD-specific antibodies from immunized mice, MAXIsorp (ThermoFisher) 96-well plates were coated directly with eOD antigen at 2 μg/ml in PBS overnight at 4° C. To capture RBD-specific antibodies, Costar Polystyrene High Binding 96-well plates (Corning) were coated directly with RBD antigen at 2 μg/ml in PBS overnight at 4° C. Plates were then blocked with PBS+2% BSA for 2 hr at 25° C. Mouse sera were diluted in block buffer (PBS+2% BSA) starting at 1:100 or 1:200, while mucosal samples were diluted in block buffer starting at 1:10, followed by 4× serial dilutions. For eOD ELISAs, VRC01 at 5 μg/ml was used as a positive control; for RBD ELISAs, mAb CR3022 or Fc-fusion protein ACE2-Fc at 5 μg/ml were used as positive controls. Samples were incubated in plates for 2 hr at 25° C., followed by detection with 1:5000 goat anti-mouse IgG-HRP (BioRad) or 1:2000 goat anti-mouse IgA-HRP (Invitrogen) in block buffer for 1 hr. Plates were developed using TMB substrate for 1-20 min and stopped with 2N sulfuric acid, and the resulting absorbance (A₄₅₀/A₅₄₀) was measured on a plate reader. For all titer analyses, samples directly compared across groups were developed for the same amount of time. Cut-off titers are reported as inverse dilutions giving an HRP absorbance (A₄₅₀-A₅₄₀) of 0.2 (RBD) or 0.1 (eOD) based on background.

ELISPOT analysis of mouse plasma cells. IgG and IgA plasma cells were analyzed in BM and FRT tissue at 35 or 52+ weeks post-prime, as indicated, using PVDF-MSIP filter plates (0.45 μm High Protein Binding Immobilon-P Membrane filter plates, Millipore) and Mouse IgG/A ELISpot-BASIC kits (Mabtech). To quantify eOD antigen-specific IgG and IgA plasma cells, filter plates were coated with 10 μg/ml eOD in 100 μl sterile PBS and incubated overnight at 4° C.; cells were plated at 500,000 and 250,000 cells/well in 100 μl cRPMI. To quantify total IgG and IgA plasma cells, filter plates were coated with 15 μg/ml anti-IgG (purified goat anti-mouse IgG capture antibody, Mabtech) or anti-IgA (monoclonal antibody MT45A, Mabtech), respectively, in 100 μl sterile PBS and incubated overnight at 4° C.; cells were plated at 100,000 and 50,000 cells/well in 100 μl cRPMI. Plates were then incubated for 18-20 h at 37° C., spot detection was carried out per manufacturer instructions, and plates were read on a CTL ImmunoSpot Analyzer.

Mouse parental control immunization. To compare intranasal immunization to a parenteral control, BALB/c mice were immunized intranasally or subcutaneously at the scruff of the neck with 5 μg amph-eOD combined with 25 μg cdGMP. Mice were primed on day 0 and boosted on day 42. Blood, vaginal, and fecal samples were collected at regular intervals as described above.

ELISA for anti-PEG antibodies. Antibody responses to PEG included in the amph-protein conjugates was assayed by ELISA. Briefly, MaxiSorp ELISA plates were coated with streptavidin at 1 μg/mL in PBS for 4 hours at 25° C., blocked with PBS+2% bovine serum albumin (BSA) overnight at 4° C., then washed three times with wash buffer (PBS containing 0.2% Tween20). Biotin-PEG-OH (Creative PEGWorks, cat. #PJK-1946) was added to the plates in blocking buffer (1 μg/mL) and incubated for 2 hours at 25° C. After washing plates three times with wash buffer, mouse serum samples and mouse anti-PEG IgG standard antibody (AffinityImmuno kit cat. #EL-141-PEG-mIGG, starting at 1 μg/ml followed by 2× serial dilutions) were added and incubated for 2 hours prior to washing. Anti-mouse IgG-HRP diluted 1:5000 in blocking buffer was used as a detection antibody. Samples were incubated for 1 hour at 25° C. before washing and adding TMB substrate, followed by 2 N H₂SO₄ as a stop solution. Absorbance was measured at 450 nm.

ACE2:RBD binding inhibition assay. Functional antibody inhibition of ACE2:RBD binding was measured in mouse serum and BALF as a preliminary indication of neutralizing antibodies using SARS-CoV-2 Surrogate Virus Neutralization Test Kits (Genscript), per manufacturer instructions. Mouse serum was diluted starting at 1:10 while BALF was diluted 1:2, followed by 4× serial dilutions. Inhibition (IC50) was defined as the sample dilution at which 50% reduction in ACE2:RBD binding was observed relative to a negative control (no inhibition).

Pseudovirus-based SARS-CoV-2 neutralization assay. The SARS-CoV-2 pseudoviruses expressing a luciferase reporter gene were generated in an approach similar to those described previously [89, 90]. Briefly, the packaging plasmid psPAX2 (AIDS Resource and Reagent Program), luciferase reporter plasmid pLenti-CMV Puro-Luc (Addgene), and spike protein expressing pcDNA3.1-SARS CoV-2 SACT were co-transfected into HEK293T cells by lipofectamine 2000 (ThermoFisher). The supernatants containing the pseudotype viruses were collected 48 h post-transfection, which were purified by centrifugation and filtration with 0.45 μm filter. To determine the neutralization activity of mouse serum and mucosal samples, HEK293ThACE2 cells were seeded in 96-well tissue culture plates at a density of 1.75×10⁴ cells/well overnight. Samples (serum, saliva, nasal wash, vaginal wash, fecal wash, and BALF) were first heat-inactivated at 56° C. for 30 min. Three-fold serial dilutions of heat-inactivated serum or mucosal samples were then prepared and mixed with 50 μL of pseudovirus. The mixture was incubated at 37° C. for 1 h before adding to HEK293T-hACE2 cells. 48 h after infection, cells were lysed in Steady-Glo Luciferase Assay (Promega) according to the manufacturer's instructions. SARS-CoV-2 neutralization titers (NT50) were defined as the sample dilution at which a 50% reduction in relative light unit (RLU) was observed relative to the average of the virus control wells.

### NHP Studies

Animals. Six female Indian rhesus macaques (Macaca mulatta) were assigned to the IVIS trafficking study (n=3 animals per group). Twelve female Indian rhesus macaques between 3-4 years of age were assigned to the longitudinal immunization study (n=6 animals per group). Macaques were distributed such that age, weight, and MHC genotyping were equivalent across groups. Animals were housed and maintained at the New Iberia Research Center (NIRC) of the University of Louisiana at Lafayette in accordance with the rules and regulations of the “Guide for the Care and Use of Laboratory Animals”. The entire study (protocol 8789-08) was reviewed and approved by the University of Louisiana at Lafayette Institutional Animal Care and Use Committee (IACUC). All animals were negative for SIV, simian T cell leukemia virus and simian retrovirus. The animals were also typed for MEW and those expressing the MamuB*008 or B*017 alleles were excluded while those expressing the MamuA*001 allele were distributed equally among the groups.

IVIS trafficking. In vivo trafficking of AF647-labeled amph-eOD and eOD was evaluated following intranasal administration using an IVIS fluorescence imaging system (Perkin Elmer). Macaques were immunized intranasally in a dropwise manner directly to each nostril, 200 μl per nare (400 μl total per animal), with 100 μg AF647-amph-eOD or AF647-eOD mixed with 375 μg SMNP. Post-administration, animals remained in the supine position under anesthesia for 10 minutes to allow for vaccine uptake and to prevent drainage. After 24 h, the tonsils, adenoids, cervical LNs, axillary LNs, and nasal tissue including turbinates were collected, fixed in 4% paraformaldehyde for 5 days, then transferred to PBS+0.1% PFA+0.05% sodium azide for storage at 4° C. prior to evaluation by IVIS.

Immunization study and sample collection. Animals were immunized intranasally at weeks 0, 8, 16, and 24 with 100 μg amph-eOD or eOD mixed with 375 μg SMNP, as described above. For longitudinal immune monitoring, peripheral blood mononuclear cells (PBMCs) were collected by venipuncture from the femoral vein, then Ficoll-separated and cryopreserved except for those used freshly for plasma blast ELISPOT assay. Serum samples were stored at −80° C. until ELISA analysis. Mucosal samples were collected by using Merocel sponges and processed as previously described [91] and stored at −80° C. until analysis.

ELISA analysis of NHP antibody titers. To measure eOD-specific antibody titers, MAXlsorp 96-well plates (ThermoFisher) were coated with 2 μg/mL of gp120 eOD monomer in PBS. Serum samples were diluted 1:50 and mucosal washes were diluted 1:10 in 2% BSA block buffer, followed by 4× serial dilutions. hVRC01 at 5 μg/ml was included as a positive control. Samples were incubated for 2 hr at RT, followed by detection with 1:5000 goat anti-human IgG-HRP (Jackson ImmunoResearch) or 1:2000 goat-anti-human IgA-HRP (ThermoFisher Scientific). Cutoff titers are reported as inverse dilutions giving an HRP absorbance (A₄₅₀-A₅₄₀) of 0.2 (IgA) or 0.1 (IgG) based on background.

ELISPOT analysis of NHP plasma cells. Total and antigen-specific plasma blast responses in peripheral blood were determined by ELISPOT assay as previously described [92]. Briefly, 96-well multiscreen HTS filter plates (Millipore) were coated overnight at 4° C. with 100 μl/well of 5 μg/ml of goat anti-monkey IgG, IgM or IgA antibodies (Rockland) or of 1 μg/ml of HIV eOD-gp120, respectively. Plates were washed with PBS-0.05% Tween 20 (PBS-T) and blocked with complete medium at 37° C. for 2 hours. Freshly isolated cells were plated in duplicates in serial 3-fold dilutions and incubated overnight in a 5% CO2 incubator at 37° C. Plates were washed with PBS-T and incubated with biotin-conjugated anti-monkey IgG, IgM, or IgA antibodies (Rockland) diluted 1:1,000 for 1 hour at 37° C. After washing, plates were incubated with horseradish peroxidase (HRP)-conjugated streptavidin diluted 1:1,000 (Vector labs) at room temperature for 2 hours and developed using the AEC substrate kit (BD Biosciences). To stop the reaction, plates were washed extensively with water followed by air drying. Spots were imaged and counted using a Immunospot ELISPOT Analyzer (Cellular Technology Limited). The number of spots specific for each Ig isotype was reported as the number of either total or antigen-specific antibody producing cells per million PBMCs.

### Statistical Analysis

Statistics were analyzed using GraphPad Prism software. For comparison of more than two groups, one- or two-way ANOVA was performed with α=0.05, followed by Tukey's or Sidak's posthoc test as indicated. For comparison of two groups, two-tailed unpaired t-test was performed with α=0.05. Statistical significance in amphiphile membrane insertion experiments was determined using simple linear regression, evaluating the dependence of AF647 or VRC01 MFI on eOD concentration to determine significant non-zero slope. ACE2:RBD binding inhibition (IC50) was determined using sigmoidal 4PL nonlinear regression. All graphs represent mean±standard error of the mean (s.e.m.) unless otherwise noted. Statistical significance is marked as *p<0.05, **p<0.01, ***p<0.001, ****p<0.0001.

### Equivalents

It is understood that the detailed examples and embodiments described herein are given by way of example for illustrative purposes only, and are in no way considered to be limiting to the invention. Various modifications or changes in light thereof will be suggested to persons skilled in the art and are included within the spirit and purview of this application and are considered within the scope of the appended claims. For example, the relative quantities of the ingredients may be varied to optimize the desired effects, additional ingredients may be added, and/or similar ingredients may be substituted for one or more of the ingredients described. Additional advantageous features and functionalities associated with the systems, methods, and processes of the present invention will be apparent from the appended claims. Moreover, those skilled in the art will recognize, or be able to ascertain using no more than routine experimentation, many equivalents to the specific embodiments of the invention described herein. Such equivalents are intended to be encompassed by the following claims.

### BIBLIOGRAPHY

1. M. R. Neutra, P. A. Kozlowski, Mucosal vaccines: the promise and the challenge, Nat Rev Immunol 6, 148-158 (2006).
2. J. Holmgren, C. Czerkinsky, Mucosal immunity and vaccines, Nat Med 11, S45-S53 (2005).
3. N. Lycke, Recent progress in mucosal vaccine development: potential and limitations, Nat Rev Immunol 12, 592-605 (2012).
4. J. R. McGhee, J. Mestecky, M. T. Dertzbaugh, J. H. Eldridge, M. Hirasawa, H. Kiyono, The mucosal immune system: from fundamental concepts to vaccine development, Vaccine 75-88 (1992).
5. S. Mitragotri, Immunization without needles, Nat Rev Immunol 5, 905-916 (2005).
6. D. Mostaghimi, C. N. Valdez, H. T. Larson, C. C. Kalinich, A. Iwasaki, Prevention of host-to-host transmission by SARS-CoV-2 vaccines, Lancet Infect Dis (2021), doi:10.1016/s1473-3099(21)00472-2.
7. J. R. McGhee, A mucosal gateway for vaccines, Nat Biotechnol 29, 136-138 (2011).
8. B. Corthesy, Secretory immunoglobulin A: well beyond immune exclusion at mucosal surfaces, Immunopharm Immunot 31, 174-179 (2008).
9. M. Zhou, R. M. Ruprecht, Are anti-HIV IgAs good guys or bad guys?, Retrovirology 11, 109 (2014).
10. M. Ali, M. Emch, L. von Seidlein, M. Yunus, D. A. Sack, M. Rao, J. Holmgren, J. D. Clemens, Herd immunity conferred by killed oral cholera vaccines in Bangladesh: a reanalysis, Lancet 366, 44-49 (2005).
11. M. Bomsel, D. Tudor, A.-S. Drillet, A. Alfsen, Y. Ganor, M.-G. Roger, N. Mouz, M. Amacker, A. Chalifour, L. Diomede, G. Devillier, Z. Cong, Q. Wei, H. Gao, C. Qin, G.-B. Yang, R. Zurbriggen, L. Lopalco, S. Fleury, Immunization with HIV-1 gp41 Subunit Virosomes Induces Mucosal Antibodies Protecting Nonhuman Primates against Vaginal SHIV Challenges, Immunity 34, 269-280 (2011).
12. A. M. Sholukh, J. D. Watkins, H. K. Vyas, S. Gupta, S. K. Lakhashe, S. Thorat, M. Zhou, G. Hemashettar, B. C. Bachler, D. N. Forthal, F. Villinger, Q. J. Sattentau, R. A. Weiss, G. Agatic, D. Corti,
A. Lanzavecchia, J. L. Heeney, R. M. Ruprecht, Defense-in-depth by mucosally administered anti-HIV dimeric IgA2 and systemic IgG1 mAbs: Complete protection of rhesus monkeys from mucosal SHIV challenge, Vaccine 33, 2086-2095 (2015).
13. D. Sterlin, A. Mathian, M. Miyara, A. Mohr, F. Anna, L. Claër, P. Quentric, J. Fadlallah, H. Devilliers, P. Ghillani, C. Gunn, R. Hockett, S. Mudumba, A. Guihot, C.-E. Luyt, J. Mayaux, A. Beurton, S. Fourati, T. Bruel, O. Schwartz, .-M. Lacorte, H. Yssel, C. Parizot, K. Dorgham, P. Charneau, Z. Amoura, G. Gorochov, IgA dominates the early neutralizing antibody response to SARS-CoV-2, Sci Transl Med 13, eabd2223 (2021).
14. Z. Wang, J. C. C. Lorenzi, F. Muecksch, S. Finkin, C. Viant, C. Gaebler, M. Cipolla, H.-H. Hoffman, T. Y. Oliveira, D. A. Oren, V. Ramos, L. Nogueira, E. Michailidis, D. F. Robbiani, A. Gazumyan, C. M. Rice, T. Hatziioannou, P. D. Bieniasz, M. Caskey, M. C. Nussenzweig, Enhanced SARS-CoV-2 neutralization by dimeric IgA, Sci Transl Med 13, eabf1555 (2021).
15. P. Brandtzaeg, Potential of Nasopharynx-associated Lymphoid Tissue for Vaccine Responses in the Airways, Am J Resp Crit Care 183, 1595-1604 (2011).
16. H. Kiyono, S. Fukuyama, NALT-versus PEYER'S-patch-mediated mucosal immunity, Nat Rev Immunol 4, 699-710 (2004).
17. E. J. Kunkel, E. C. Butcher, Plasma-cell homing, Nat Rev Immunol 3, 822-829 (2003).
18. M. Li, Y. Wang, Y. Sun, H. Cui, S. J. Zhu, H.-J. Qiu, Mucosal vaccines: Strategies and challenges, Immunol Lett 217, 116-125 (2019).
19. Y. L. Mato, Nasal route for vaccine and drug delivery: features and current opportunities, Int J Pharmaceut 572, 118813 (2019).
20. M. R. Neutra, N. J. Mantis, J.-P. Kraehenbuhl, Collaboration of epithelial cells with organized mucosal lymphoid tissues, Nat Immunol 2,1004-1009 (2001).
21. C. Czerkinsky, J. Holmgren, Vaccines against enteric infections for the developing world, Philosophical Transactions Royal Soc B Biological Sci 370, 20150142 (2015).
22. A. Miguel-Clopés, E. G. Bentley, J. P. Stewart, S. R. Carding, Mucosal vaccines and technology, Clin Exp Immunol 196, 205-214 (2019).
23. J. R. Simard, P. A. Zunszain, C.-E. Ha, J. S. Yang, N. V. Bhagavan, I. Petitpas, S. Curry, J. A. Hamilton, Locating high-affinity fatty acid-binding sites on albumin by x-ray crystallography and NMR spectroscopy, P Natl Acad Sci Usa 102, 17958-17963 (2005).
24. E. N. Hoogenboezem, C. L. Duvall, Harnessing albumin as a carrier for cancer therapies, Advanced Drug Delivery Reviews 130, 73-89 (2018).
25. H. Liu, K. D. Moynihan, Y. Zheng, G. L. Szeto, A. V. Li, B. Huang, D. S. V. Egeren, C. Park, D. J.
Irvine, Structure-based programming of lymph-node targeting in molecular vaccines, Nature, 1-(2014).
26. L. Ma, T. Dichwalkar, J. Y. H. Chang, B. Cossette, D. Garafola, A. Q. Zhang, M. Fichter, C. Wang, S. Liang, M. Silva, S. Kumari, N. K. Mehta, W. Abraham, N. Thai, N. Li, K. D. Wittrup, D. J. Irvine, Enhanced CAR-T cell activity against solid tumors by vaccine boosting through the chimeric receptor., Sci New York N Y 365, 162-168 (2019).
27. K. D. Moynihan, R. L. Holden, N. K. Mehta, C. Wang, M. R. Karver, J. Dinter, S. Liang, W. Abraham, M. B. Melo, A. Q. Zhang, N. Li, S. L. Gall, B. L. Pentelute, D. J. Irvine, Enhancement of Peptide Vaccine Immunogenicity by Increasing Lymphatic Drainage and Boosting Serum Stability., Cancer Immunology Research 6, 1025-1038 (2018).
28. K. D. Moynihan, C. F. Opel, G. L. Szeto, A. Tzeng, E. F. Zhu, J. M. Engreitz, R. T. Williams, K. Rakhra, M. H. Zhang, A. M. Rothschilds, S. Kumari, R. L. Kelly, B. H. Kwan, W. Abraham, K. Hu, N. K. Mehta, M. J. Kauke, H. Suh, J. R. Cochran, D. A. Lauffenburger, K. D. Wittrup, D. J. Irvine, Eradication of large established tumors in mice by combination immunotherapy that engages innate and adaptive immune responses, Nat Med 22, 1402-1410 (2016).
29. K. Rakhra, W. Abraham, C. Wang, K. D. Moynihan, N. Li, N. Donahue, A. D. Baldeon, D. J. Irvine, Exploiting albumin as a mucosal vaccine chaperone for robust generation of lung-resident memory T cells, Sci Immunol 6, eabd8003 (2021).
30. J. T. Sockolosky, F. C. Szoka, The neonatal Fc receptor, FcRn, as a target for drug delivery and therapy, Adv Drug Deliver Rev 91, 109-124 (2015).
31. K. M. K. Sand, M. Bern, J. Nilsen, H. T. Noordzij, I. Sandlie, J. T. Andersen, Unraveling the Interaction between FcRn and Albumin: Opportunities for Design of Albumin-Based Therapeutics, Front Immunol 5, 682 (2015).
32. C. L. Anderson, C. Chaudhury, J. Kim, C. L. Bronson, M. A. Wani, S. Mohanty, Perspective—FcRn transports albumin: relevance to immunology and medicine, Trends Immunol 27, 343-348 (2006).
33. K. Baker, S.-W. Qiao, T. Kuo, K. Kobayashi, M. Yoshida, W. I. Lencer, R. S. Blumberg,

Immune and non-immune functions of the (not so) neonatal Fc receptor, FcRn, Semin Immunopathol 31, 223-236 (2009).
34. D. C. Roopenian, S. Akilesh, FcRn: the neonatal Fc receptor comes of age, Nat Rev Immunol 7, 715-725 (2007).
35. E. S. Ward, R. J. Ober, HHS Public Access, Adv Immunol 103, 77-115 (2009).
36. A. J. Pollard, E. M. Bijker, A guide to vaccinology: from basic principles to new developments, Nat Rev Immunol 21, 83-100 (2021).
37. S. A. Plotkin, Correlates of protection induced by vaccination., Clinical and vaccine immunology: CVI 17, 1055-1065 (2010).
38. J. Jardine, J.-P. Julien, S. Menis, T. Ota, O. Kalyuzhniy, A. McGuire, D. Sok, P.-S. Huang, S. MacPherson, M. Jones, T. Nieusma, J. Mathison, D. Baker, A. B. Ward, D. R. Burton, L. Stamatatos, D. Nemazee, I. A. Wilson, W. R. Schief, Rational HIV Immunogen Design to Target Specific Germline B Cell Receptors, Science 340, 711-716 (2013).
39. J. G. Jardine, T. Ota, D. Sok, M. Pauthner, D. W. Kulp, O. Kalyuzhniy, P. D. Skog, T. C. Thinnes, D. Bhullar, B. Briney, S. Menis, M. Jones, M. Kubitz, S. Spencer, Y. Adachi, D. R. Burton, W. R. Schief, D. Nemazee, Priming a broadly neutralizing antibody response to HIV-1 using a germline-targeting immunogen, Science 349, 156-161 (2015).
40. J. G. Jardine, D. W. Kulp, C. Havenar-Daughton, A. Sarkar, B. Briney, D. Sok, F. Sesterhenn, J. Ereño-Orbea, O. Kalyuzhniy, I. Deresa, X. Hu, S. Spencer, M. Jones, E. Georgeson, Y. Adachi, M. Kubitz, A. C. deCamp, J.-P. Julien, I. A Wilson, D. R. Burton, S. Crotty, W. R. Schief, HIV-1 broadly neutralizing antibody precursor B cells revealed by germline-targeting immunogen, Science 351, 1458-1463 (2016).
41. D. Sok, B. Briney, J. G. Jardine, D. W. Kulp, S. Menis, M. Pauthner, A. Wood, E.-C. Lee, K. M. Le, M. Jones, A. Ramos, O. Kalyuzhniy, Y. Adachi, M. Kubitz, S. MacPherson, A. Bradley, G. A. Friedrich, W. R. Schief, D. R. Burton, Priming HIV-1 broadly neutralizing antibody precursors in human Ig loci transgenic mice, Science 353, 1557-1560 (2016).
42. C. L. Anderson, C. Chaudhury, J. Kim, C. L. Bronson, M. A. Wani, S. Mohanty, Perspective—FcRn transports albumin: relevance to immunology and medicine., TRENDS in Immunology 27, 343-348 (2006).
43. M. Bern, J. Nilsen, M. Ferrarese, K. M. K. Sand, T. T. Gjolberg, H. E. Lode, R. J. Davidson, R. M. Camire, E. S. Bækkevold, S. Foss, A. Grevys, B. Dalhus, J. Wilson, L. S. Høydahl, G. J. Christianson, D. C. Roopenian, T. Schlothauer, T. E. Michaelsen, M. C. Moe, S. Lombardi, M. Pinotti, I. Sandlie, A. Branchini, J. T. Andersen, An engineered human albumin enhances half-life and transmucosal delivery when fused to protein-based biologics, Sci Transl Med 12, eabb0580 (2020).
44. S. M. Blaauboer, V. D. Gabrielle, L. Jin, MPYS/STING-Mediated TNF-, Not Type I IFN, IsEssential for the Mucosal Adjuvant Activity of (3′-5′)-Cyclic-Di-Guanosine-Monophosphate In Vivo, 192, 492-502 (2013).
45. S. M. Blaauboer, S. Mansouri, H. R. Tucker, H. L. Wang, The mucosal adjuvant cyclic di-GMP enhances antigen uptake and selectively activates pinocytosis-efficient cells in vivo, eLife (2015), doi:10.7554/elife.06670.001.
46. T. Ebensen, K. Schulze, P. Riese, M. Morr, C. A. Guzmán, The bacterial second messenger cdiG1VIP exhibits promising activity as a mucosal adjuvant., Clinical and Vaccine Immunology 14, 952-958 (2007).
47. M. Silva, Y. Kato, M. B. Melo, I. Phung, B. L. Freeman, Z. Li1, 7 Sonya Haupt 3, 5 Kangsan Roh6, J. W. V. Wijnbergen6, H. Watkins1, C. A. Enemuo7, B. L. 6 Hartwell1, J. Y. H. Chang1, S. Xiao1, K. A . Rodrigues1, K. M. Cirelli2, N. Li1, A. Aung1, B. Cossette1, W. Abraham1, S. Kataria1, R. 8 Bastidas2, J. Bhiman2, C. Linde4, N. I. Bloom3, B. Groschel2, E. 9 Georgeson2, N. Phelps2, A. Thomas1, J. Bals4, D. G. Carnathan2, D. 10 Lingwood4, D. R. Burton2, G. Alter4, T. P. Padera6, A. M. Belcher1, W. R. 11 Schief2, G. Silvestri2, R. M. Ruprecht13, S. Crotty2, and D. J. Irvine, A potent saponin and TLR agonist particulate vaccine adjuvant alters lymphatic flow and modulates B and T cell responses.
48. P. T. Heath, E. P. Galiza, D. N. Baxter, M. Boffito, D. Browne, F. Burns, D. R. Chadwick, R. Clark, C. Cosgrove, J. Galloway, A. L. Goodman, A. Heer, A. Higham, S. Iyengar, A. Jamal, C. Jeanes, P. A. Kalra, C. Kyriakidou, D. F. McAuley, A. Meyrick, A. M. Minassian, J. Minton, P. Moore, I. Munsoor, H. Nicholls, O. Osanlou, J. Packham, C. H. Pretswell, A. S. F. Ramos, D. Saralaya, R. P. Sheridan, R. Smith, R. L. Soiza, P. A. Swift, E. C. Thomson, J. Turner, M. E. Viljoen, G. Albert, I. Cho, F. ubovsky, G. Glenn, J. Rivers, A. Robertson, K. Smith, S. Toback, 2019nCoV-302 Study Group, Safety and Efficacy of NVX-CoV2373 Covid-19 Vaccine, New Engl J Med (2021), doi:10.1056/nejmoa2107659.
49. M. T. Sanders, G. Deliyannis, M. J. Pearse, M. K. McNamara, L. E. Brown, Single dose intranasal immunization with ISCOMATRIX vaccines to elicit antibody-mediated clearance of influenza virus requires delivery to the lower respiratory tract., Vaccine 27, 2475-2482 (2009).
50. A. Vujanic, J. L. K. Wee, K. J. Snibson, S. Edwards, M. Pearse, C. Quinn, M. Moloney, S. Taylor, J.-P. Y. Scheerlinck, P. Sutton, Combined mucosal and systemic immunity following pulmonary delivery of ISCOMATRIX™ adjuvanted recombinant antigens, Vaccine 28, 2593-2597 (2010).
51. L. Moreno-Fierros, I. Garcia-Silva, S. Rosales-Mendoza, Development of SARS-CoV-2 vaccines: should we focus on mucosal immunity?, Expert Opin Biol Th 20, 1-6 (2020).
52. G. Dagotto, J. Yu, D. H. Barouch, Approaches and Challenges in SARS-CoV-2 Vaccine Development, Cell Host Microbe 28, 364-370 (2020).
53. F. E. Lund, T. D. Randall, Scent of a vaccine,Science 373, 397-399 (2021).
54. F. Krammer, SARS-CoV-2 vaccines in development, Nature, 1-12 (2020).
55. L. Dai, T. Zheng, K. Xu, Y. Han, L. Xu, E. Huang, Y. An, Y. Cheng, S. Li, M. Liu, M. Yang, Y. Li, H. Cheng, Y. Yuan, W. Zhang, C. Ke, G. Wong, J. Qi, C. Qin, J. Yan, G. F. Gao, A Universal Design of Betacoronavirus Vaccines against COVID-19, MERS, and SARS, Cell 182, 722-733.ell (2020).
56. J. Yang, W. Wang, Z. Chen, S. Lu, F. Yang, Z. Bi, L. Bao, F. Mo, X. Li, Y. Huang, W. Hong, Y. Yang, Y. Zhao, F. Ye, S. Lin, W. Deng, H. Chen, H. Lei, Z. Zhang, M. Luo, H. Gao, Y. Zheng, Y. Gong, X. Jiang, Y. Xu, Q. Lv, D. Li, M. Wang, F. Li, S. Wang, G. Wang, P. Yu, Y. Qu, L. Yang, H. Deng, A. Tong, J. Li, Z. Wang, J. Yang, G. Shen, Z. Zhao, Y. Li, J. Luo, H. Liu, W. Yu, M. Yang, J. Xu, J. Wang, H. Li, H. Wang, D. Kuang, P. Lin, Z. Hu, W. Guo, W. Cheng, Y. He, X. Song, C. Chen, Z. Xue, S. Yao, L. Chen, X. Ma, S. Chen, M. Gou, W. Huang, Y. Wang, C. Fan, Z. Tian, M. Shi, F.-S. Wang, L. Dai, M. Wu, G. Li, G. Wang, Y. Peng, Z. Qian, C. Huang, J. Y.-N. Lau, Z. Yang, Y. Wei, X. Cen, X. Peng, C. Qin, K. Zhang, G. Lu, X. Wei, A vaccine targeting the RBD of the S protein of SARS-CoV-2 induces protective immunity, Nature 586, 572-577 (2020).
57. N. C. Dalvie, S. A. Rodriguez-Aponte, B. L. Hartwell, L. H. Tostanoski, A. M. Biedermann, L. E. Crowell, K. Kaur, O. S. Kumru, L. Carter, J. Yu, A. Chang, K. McMahan, T. Courant, C. Lebas, A. A. Lemnios, K. A. Rodrigues, M. Silva, R. S. Johnston, C. A. Naranjo, M. K. Tracey, J. R. Brady, C. A. Whittaker, D. Yun, N. Brunette, J. Y. Wang, C. Walkey, B. Fiala, S. Kar, M. Porto, M. Lok, H. Andersen, M. G. Lewis, K. R. Love, D. L. Camp, J. M. Silverman, H. Kleanthous, S. B. Joshi, D. B. Volkin, P. M. Dubois, N. Collin, N. P. King, D. H. Barouch, D. J. Irvine, J. C. Love, Engineered SARS-CoV-2 receptor binding domain improves manufacturability in yeast and immunogenicity in mice, Proc National Acad Sci 118, e2106845118 (2021).
58. J. R. Harkema, S. A. Carey, J. G. Wagner, S. M. Dintzis, D. Liggitt, Comparative Anatomy and Histology, 71-94 (2012).
59. P. Brandtzaeg, Function of Mucosa-Associated Lymphoid Tissue in Antibody Formation, Immunol Invest 39, 303-355 (2010).
60. J. R. Harkema, S. A. C. and J. G. Wagner, S. M. D. and D. Liggitt, in Nose, Sinus, Pharynx, and Larynx, Elsevier, Ed. (2012), pp. 71-94.
61. M. R. Neutra, N. J. Mantis, J.-P. Kraehenbuhl, Collaboration of epithelial cells with organized mucosal lymphoid tissues, Nat Immunol 2, 1004-1009 (2001).
62. A. Silva-Sanchez, T. D. Randall, Mucosal Vaccines, 21-54 (2020).
63. S. Kimura, Molecular insights into the mechanisms of M-cell differentiation and transcytosis in the mucosa-associated lymphoid tissues, Anat Sci Int 93, 23-34 (2018).
64. D.-Y. Kim, A. Sato, S. Fukuyama, H. Sagara, T. Nagatake, I. G. Kong, K. Goda, T. Nochi, J. Kunisawa, S. Sato, Y. Yokota, C. H. Lee, H. Kiyono, The Airway Antigen Sampling System: Respiratory M Cells as an Alternative Gateway for Inhaled Antigens, J Immunol 186, 4253-4262 (2011).
65. A. Dillon, D. D. Lo, M Cells: Intelligent Engineering of Mucosal Immune Surveillance, Front Immunol 10, 1499 (2019).
66. K. Baker, T. Rath, M. Pyzik, R. S. Blumberg, The Role of FcRn in Antigen Presentation, Front Immunol 5, 408 (2014).
67. D. C. Roopenian, S. Akilesh, FcRn: the neonatal Fc receptor comes of age, Nat Rev Immunol 7,715-725 (2007).
68. Transepithelial transport of Fc-targeted nanoparticles by the neonatal fc receptor for oral delivery., Science Translational Medicine 5, 213ra167-213ra167 (2013).
69. A. J. Bitonti, J. A. Dumont, S. C. Low, R. T. Peters, K. E. Kropp, V. J. Palombella, J. M. Stattel, Y. Lu, C. A. Tan, J. J. Song, A. M. Garcia, N. E. Simister, G. M. Spiekermann, W. I. Lencer, R. S. Blumberg, Pulmonary delivery of an erythropoietin Fc fusion protein in non-human primates through an immunoglobulin transport pathway, P Natl Acad Sci Usa 101, 9763-9768 (2004).
70. J. A. Dumont, A. J. Bitonti, D. Clark, S. Evans, M. Pickford, S. P. Newman, Delivery of an Erythropoietin-Fc Fusion Protein by Inhalation in Humans through an Immunoglobulin Transport Pathway, J Aerosol Medicine 18, 294-303 (2005).
71. E. M. Pridgen, F. Alexis, T. T. Kuo, E. Levy-Nissenbaum, R. Karnik, R. S. Blumberg, R. Langer, O. C. Farokhzad, Transepithelial Transport of Fc-Targeted Nanoparticles by the Neonatal Fc Receptor for Oral Delivery, Sci Transl Med 5, 213ra167-213ra167 (2013).
72. L. Ye, R. Zeng, Y. Bai, D. C. Roopenian, X. Zhu, Efficient mucosal vaccination mediated by the neonatal Fc receptor, Nat Biotechnol 29, 158-163 (2011).
73. L. Lu, S. Palaniyandi, R. Zeng, Y. Bai, X. Liu, Y. Wang, C. D. Pauza, D. C. Roopenian, X. Zhu, A Neonatal Fc Receptor-Targeted Mucosal Vaccine Strategy Effectively Induces HIV-1 Antigen-Specific Immunity to Genital Infection, J Virol 85, 10542-10553 (2011).
74. N. K. Mehta, R. V. Pradhan, A. P. Soleimany, K. D. Moynihan, A. M. Rothschilds, N. Momin, K. Rakhra, J. Mata-Fink, S. N. Bhatia, K. D. Wittrup, D. J. Irvine, Pharmacokinetic tuning of protein-antigen fusions enhances the immunogenicity of T-cell vaccines, Nat Biomed Eng 4, 636-648 (2020).
50. L. Dai, G. F. Gao, Viral targets for vaccines against COVID-19, Nat Rev Immunol 21, 73-82 (2021).
76. A. O. Hassan, N. M. Kafai, I. P. Dmitriev, J. M. Fox, B. K. Smith, I. B. Harvey, R. E. Chen, E. S. Winkler, A. W. Wessel, J. B. Case, E. Kashentseva, B. T. McCune, A. L. Bailey, H. Zhao, L. A. VanBlargan, Y.-N. Dai, M. Ma, L. J. Adams, S. Shrihari, J. E. Danis, L. E. Gralinski, Y. J. Hou, A. Schäfer, A. S. Kim, S. P. Keeler, D. Weiskopf, R. S. Baric, M. J. Holtzman, D. H. Fremont, D. T. Curiel, M. S. Diamond, A Single-Dose Intranasal ChAd Vaccine Protects Upper and Lower Respiratory Tracts against SARS-CoV-2, Cell 183, 169-184.e13 (2020).
77. N. Lycke, Recent progress in mucosal vaccine development: potential and limitations, Nature Reviews Immunology 12, 592-605 (2012).
78. K. Imaoka, C. J. Miller, M. Kubota, M. B. McChesney, B. Lohman, M. Yamamoto, K. Fujihashi, K. Someya, M. Honda, J. R. McGhee, H. Kiyono, Nasal immunization of nonhuman primates with simian immunodeficiency virus p55gag and cholera toxin adjuvant induces Th1/Th2 help for virus-specific immune responses in reproductive tissues., J Immunol Baltim Md 1950 161, 5952-8 (1998).
79. Y. Enose, M. Ui, A. Miyake, H. Suzuki, H. Uesaka, T. Kuwata, J. Kunisawa, H. Kiyono, H. Takahashi, T. Miura, M. Hayami, Protection by Intranasal Immunization of a nef-Deleted, Nonpathogenic SHIV against Intravaginal Challenge with a Heterologous Pathogenic SHIV, Virology 298, 306-316 (2002).
80. M. Vajdy, M. Singh, J. Kazzaz, E. Soenawan, M. Ugozzoli, F. Zhou, I. Srivastava, Q. Bin, S. Barnett, J. Donnelly, P. Luciw, L. Adamson, D. Montefiori, D. T. O'hagan, Mucosal and Systemic Anti-HIV Responses in Rhesus Macaques following Combinations of Intranasal and Parenteral Immunizations, Aids Res Hum Retrov 20, 1269-1281 (2004).
81. M. Bomsel, D. Tudor, A.-S. Drillet, A. Alfsen, Y. Ganor, M.-G. Roger, N. Mouz, M. Amacker, A. Chalifour, L. Diomede, G. Devillier, Z. Cong, Q. Wei, H. Gao, C. Qin, G.-B. Yang, R. Zurbriggen, L. Lopalco, S. Fleury, Immunization with HIV-1 gp41 Subunit Virosomes Induces Mucosal Antibodies Protecting Nonhuman Primates against Vaginal SHIV Challenges, Immunity 34, 269-280 (2011).
82. A. Rudin, G. C. Riise, J. Holmgren, Antibody Responses in the Lower Respiratory Tract and Male Urogenital Tract in Humans after Nasal and Oral Vaccination with Cholera Toxin B Subunit, Infect Immun 67,2884-2890 (1999).
83. C. Bergquist, E. L. Johansson, T. Lagergård, J. Holmgren, A. Rudin, Intranasal vaccination of humans with recombinant cholera toxin B subunit induces systemic and local antibody responses in the upper respiratory tract and the vagina, Infect Immun 65,2676-2684 (1997).
84. J. Jardine, J.-P. Julien, S. Menis, T. Ota, O Kalyuzhniy, A. McGuire, D. Sok, P.-S. Huang, S. MacPherson, M. Jones, T. Nieusma, J. Mathison, D. Baker, A. B. Ward, D. R. Burton, L. Stamatatos, D. Nemazee, I. A. Wilson, W. R. Schief, Rational HIV Immunogen Design to Target Specific Germline B Cell Receptors, Science 340, 711-716 (2013).
85. T. J. Moyer, Y. Kato, W. Abraham, J. Y. H. Chang, D. W. Kulp, N. Watson, H. L. Turner, S. Menis, R. K. Abbott, J. N. Bhiman, M. B. Melo, H. A. Simon, S. H.-D. la Mata, S. Liang, G. Seumois, Y. Agarwal, N. Li, D. R. Burton, A. B. Ward, W. R. Schief, S. Crotty, D. J. Irvine, Engineered immunogen binding to alum adjuvant enhances humoral immunity, Nat Med 26, 430-440 (2020).
86. T. Tokatlian, D. W. Kulp, A. A. Mutafyan, C. A. Jones, S. Menis, E. Georgeson, M. Kubitz, M. H. Zhang, M. B. Melo, M. Silva, D. S. Yun, W. R. Schief, D. J. Irvine, Enhancing Humoral Responses Against HIV Envelope Trimers via Nanoparticle Delivery with Stabilized Synthetic Liposomes, Sci Repuk 8, 16527 (2018).
87. S. M, K. Y, M. M, L. Z, C. K, F. B, R. K, W. J, W. H, X. S, H. B, C. J, A. A, C. B, B. R, B. J, L. C, T. A, B. D, A. G, P. T, B. A, R. R, S. W, C. S, I. D J, A potent saponin and TLR agonist particulate vaccine adjuvant alters lymphatic flow and lymph node antigen accumulation, Science Immunology, in press (2021).
88. E. D. Cisney, S. Fernandez, S. I. Hall, G. A. Krietz, R. G. Ulrich, Examining the Role of Nasopharyngeal-associated Lymphoreticular Tissue (NALT) in Mouse Responses to Vaccines, J Vis Exp Jove, 3960 (2012).
89. A. Chandrashekar, J. Liu, A. J. Martinot, K. McMahan, N. B. Mercado, L. Peter, L. H. Tostanoski, J. Yu, Z. Maliga, M. Nekorchuk, K. Busman-Sahay, M. Terry, L. M. Wrijil, S. Ducat, D. R. Martinez, C. Atyeo, S. Fischinger, J. S. Burke, M. D. Slein, L. Pessaint, A. V. Ry, J. Greenhouse, T. Taylor, K. Blade, A. Cook, B. Finneyfrock, R. Brown, E. Teow, J. Velasco, R. Zahn, F. Wegmann, P. Abbink, E. A. Bondzie, G. Dagotto, M. S. Gebre, X. He, C. Jacob-Dolan, N. Kordana, Z. Li, M. A. Lifton, S. H. Mahrokhian, L. F. Maxfield, R. Nityanandam, J. P. Nkolola, A. G. Schmidt, A. D. Miller, R. S. Baric, G. Alter, P. K. Sorger, J. D. Estes, H. Andersen, M. G. Lewis, D. H. Barouch, SARS-CoV-2 infection protects against rechallenge in rhesus macaques, Science 369, 812-817 (2020).
90. J. Yu, L. H. Tostanoski, L. Peter, N. B. Mercado, K. McMahan, S. H. Mahrokhian, J. P. Nkolola, J. Liu, Z. Li, A. Chandrashekar, D. R. Martinez, C. Loos, C. Atyeo, S. Fischinger, J. S. Burke, M. D. Slein, Y. Chen, A. Zuiani, F. J. N. Lelis, M. Travers, S. Habibi, L. Pessaint, A. V. Ry, K. Blade, R. Brown, A. Cook, B. Finneyfrock, A. Dodson, E. Teow, J. Velasco, R. Zahn, F. Wegmann, E. A. Bondzie, G. Dagotto, M. S. Gebre, X. He, C. Jacob-Dolan, M. Kirilova, N. Kordana, Z. Lin, L. F. Maxfield, F. Nampanya, R. Nityanandam, J. D. Ventura, H. Wan, Y. Cai, B. Chen, A. G. Schmidt, D. R. Wesemann, R. S. Baric, G. Alter, H. Andersen, M. G. Lewis, D. H. Barouch, DNA vaccine protection against SARSCoV-2 in rhesus macaques, Science 369, 806-811 (2020).
91. P. A. Kozlowski, R. M. Lynch, R. R. Patterson, S. CuUvin, T. P. Flanigan, M. R. Neutra, Modified Wick Method Using Weck-Cel Sponges for Collection of Human Rectal Secretions and Analysis of Mucosal HIV Antibody, Jaids J Acquir Immune Defic Syndromes 24, 297-309 (2000).
92. J. Wrammert, K. Smith, J. Miller, W. A. Langley, K. Kokko, C. Larsen, N.-Y. Zheng, I. Mays, L. Garman, C. Helms, J. James, G. M. Air, J. D. Capra, R. Ahmed, P. C. Wilson, Rapid cloning of high affinity human monoclonal antibodies against influenza virus, Nature 453, 667-671 (2008).

1. A vaccine comprising an amphiphilic conjugate, wherein the amphiphilic conjugate comprises an immunogen operably linked to an albumin-binding lipid, and wherein the vaccine is suitable for transmucosal administration to induce a humoral immune response.

2. The vaccine of claim 1, wherein the transmucosal administration is intranasal administration.

3. The vaccine of claim 1, wherein the immunogen is a protein antigen having a molecular weight between about 10 kDa and about 500 kDa.

4. The vaccine of claim 1, wherein the immunogen comprises a protein antigen selected from the group consisting of a human immunodeficiency virus (HIV) antigen, a SARS-CoV-2 antigen, an influenza antigen, a rotavirus antigen, a cytomegalovirus (CMV) antigen, an Epstein-Barr virus (EBV) antigen, a respiratory syncytial virus (RSV) antigen, and a cholera antigen.

5. The vaccine of claim 1, wherein the immunogen comprises a monomer antigen or trimer antigen.

6. The vaccine of claim 1, wherein the immunogen comprises an antigenic peptide.

7. The vaccine of claim 1, wherein the albumin-binding lipid is selected from the group consisting of a cholesterol, a monoacyl lipid, and a diacyl lipid.

8. (canceled)

9. The vaccine of claim 6, wherein the albumin-binding lipid is 1,2-distearoyl-sn-glycero-3-phosphoethanolamine (DSPE).

10. The vaccine of claim 1, wherein the immunogen is operably linked to the albumin-binding lipid via a first linker.

11. The vaccine of claim 10, wherein the first linker is selected from the group consisting of a hydrophilic polymer, a string of hydrophilic amino acids, polysaccharides, oligonucleotides, or a combination thereof.

12. The vaccine of claim 11, wherein the first linker comprises a polyethylene glycol (PEG) linker.

13.-14. (canceled)

15. The vaccine of claim 10, further comprising a second linker, wherein the second linker is located between the immunogen and the first linker, or between the albumin-binding lipid and the first linker.

16. The vaccine of claim 15, wherein the second linker comprises a PEG linker comprising repeating unit of PEG monomers.

17. The vaccine of claim 16, wherein the second linker comprises 2 to 20 repeating units of PEG monomers, or 4 repeating units of PEG monomers.

18. (canceled)

19. The vaccine of claim 16, wherein the second linker comprises a dibenzocyclooctyne (DBCO) group covalently conjugated to the repeating unit of PEG monomers.

20.-23. (canceled)

24. The vaccine of claim 1, further comprising an adjuvant.

25. (canceled)

26. The vaccine of claim 1, wherein transmucosal administration of the vaccine elicits or enhances production of antibodies that bind to the immunogen.

27.-28. (canceled)

29. A method of vaccinating a subject, comprising transmucosally administering to the subject an effective amount of the vaccine of claim 1, thereby vaccinating the subject.

30. A method of immunizing a subject, comprising transmucosally administering to the subject an effective amount of the vaccine of claim 1, thereby immunizing the subject.

31. The method of claim 29, wherein the vaccine is administered intranasally to the subject.

32.-34. (canceled)

35. The method of claim 29, wherein the vaccine is administered at a dose of about 5 μg to about 300 μg, or at a dose of about 50 μg, 100 μg, or 150 μg.

36. (canceled)

37. The method of claim 29, wherein the vaccine is administered in combination with an adjuvant.

38.-42. (canceled)


SEQUENCE LISTING (4 sequences)

SEQ ID NO: 1 - protein, 199 aa, synthetic construct
ETGCHHHHHHGGDTITLPCRPAPPPHCSSNITGLILTRQGGYSNDNTVIFRPSGGDWRDIARCQIAGTVVSTQLFLNGSLAEEEVVIRSEDWRDNAKSICVQLNTSVEINCTGAGHCNISRAKWNNTLKQIASKLREQYGNKTIIFKPSSGGDPEFVNHSFNCGGEFFYCDSTQLFNSTWFNSTGSAKFVAAWTLKAAA

SEQ ID NO: 2 - protein, 202 aa, synthetic construct
CITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYKYRLFRKSNLKPFERDISTEIYQAGSTPCNGVEGFNCYWPLQSYGFQPTNGVGYQPYRVVVLSFELLHAPATVCGPKKSTN

SEQ ID NO: 3 - protein, 644 aa, synthetic construct
AENLWVTVYYGVPVWKDAETTLFCASDAKAYETEKHNVWATHACVPTDPNPQEIHLENVTEEFNMWKNNMVEQMHEDIISLWDQSLKPCVKLTPLCVTLQCTNVTNNITDDMRGELKNCSFNMTTELRDKKQKVYSLFYRLDVVQINENQGNRSNNSNKEYRLINCNTSAITQACPKVSFEPIPIHYCAPAGFAILKCKDKKFNGTGPCPSVSTVQCTHGIKPVVSTQLLLNGSLAEEEVIIRSENITNNAKNILVQLNTPVQINCTRPNNNTVKSIRIGPGQAFYYTGDIIGDIRQAHCNVSKATWNETLGKVVKQLRKHFGNNTIIRFAQSSGGDLEVTTHSFNCGGEFFYCNTSGLFNSTWISNTSVQGSNSTGSNDSITLPCRIKQIINMWQRIGQAMYAPPIQGVIRCVSNITGLILTRDGGSTNSTTETFRPGGGDMRDNWRSELYKYKVVKIEPLGVAPTRCKRRVVGRRRRRRAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALDGTKHHHHHHC

SEQ ID NO: 4 - protein, 13 aa, synthetic construct
AKFVAAWTLKAAA